(19) United States
(12) Patent Application Publication
(10) Pub. No.: US 2014/0199233 A1
(43) Pub. Date: Jul. 17, 2014

(54) Enhanced Growth Inhibition of Osteosarcoma by Cytotoxic Polymerized Liposomal Nanoparticles Targeting the Alcam Cell Surface Receptor

(71) Applicants: Jon O. Nagy, Bozeman, MT (US); Noah Federman, Culver City, CA (US); Christopher Denny, Topanga, CA (US); James S. Tomlinson, Los Angeles, CA (US)

(72) Inventors: Jon O. Nagy, Bozeman, MT (US); Noah Federman, Culver City, CA (US); Christopher Denny, Topanga, CA (US); James S. Tomlinson, Los Angeles, CA (US)

(73) Assignees: THE REGENTS OF THE UNIVERSITY OF CALIFORNIA, Oakland, CA (US); NANOVALENT PHARMACEUTICALS, INC., Bozeman, MT (US)

(21) Appl. No.: 14/116,688

(22) PCT Filed: May 11, 2012
(86) PCT No.: PCT/US2012/037457

§ 371 Date: Feb. 24, 2014

Related U.S. Application Data
(60) Provisional application No. 61/485,024, filed on May 11, 2011.
(60) Provisional application No. 61/543,193, filed on Oct. 4, 2011.
(60) Provisional application No. 61/560,443, filed on Nov. 16, 2011.

(51) Int. Cl. A61K 9/51; A61K 31/704; A61K 51/12; A61K 33/24; A61K 31/519; A61K 9/127; A61K 31/513

(52) U.S. Cl. CPC A61K 9/51; A61K 9/1273; A61K 31/704; A61K 31/513; A61K 33/24; A61K 31/519; A61K 51/1244
USPC 424/1.11; 424/450; 514/34; 514/274; 424/649; 514/249

(57) ABSTRACT

The present invention relates to the fabrication and uses of liposomal nanoparticles.

### INCORPORATION BY REFERENCE

This application claims the benefit of U.S. Provisional Application 61/485,024, filed on May 11, 2011, U.S. Provisional Application 61/560,443, filed on Nov. 16, 2011, and U.S. Provisional Application 61/543,193, filed on Oct. 4, 2011, each of which are incorporated by reference herein in their entirety. All publications, patents, and patent applications mentioned in this specification are herein incorporated by reference to the same extent as if each individual publication, patent, or patent application was specifically and individually indicated to be incorporated by reference.

### STATEMENT AS TO FEDERALLY SPONSORED RESEARCH

This invention was made with government support under a Veterans Affairs Career Development Award from the Department of Veterans Affairs, and by grants CA-92865, CA-16042, and AI-28697 from the National Institutes of Health. The government has certain rights in the invention.

### BACKGROUND OF THE INVENTION

Systemic delivery of drugs to a mammal is a common practice in the treatment of disease. This form of delivery is suitable when the condition to be treated occur system wide. However, in some cases of localized diseases, such as cardiovascular diseases or cancers, providing an effective concentration to the treated site using systemic delivery of the medication can result in high drug concentrations throughout the patient. These high drug concentrations can produce adverse or toxic side effects. Thus, local delivery methods can provide much lower systemic concentrations of medication throughout the patient. This concentration difference allows local delivery to cause fewer side effects and achieve better results.

Nanotechnology and nanoparticles offer the opportunity to deliver high doses of drugs or other bioactive agents to target regions anywhere in the body, as well as limit the bystander and dose-limiting effects of such therapies on non-target tissues.

### SUMMARY OF THE INVENTION

In some embodiments, the polymerized lipid nanoparticle comprises a polymerized lipid shell, wherein the polymerized lipid shell comprises about 15% to about 20% of polymerizable lipid, about 1-15% of negatively charged lipid, about 20-45% of neutrally charged molecules (such as cholesterol) and about 30 to 60% of zwitterionically charged lipid. In some embodiments, the polymerized lipid shell comprises about 15 to about 20% of 10,12-pentacosadiynoic acid derivatives and about 30% to about 40% of saturated phospholipids. In some embodiments, the polymerized lipid shell comprises about 15% of C25 tail lipid and about 50 to about 55% of C18 tail lipid. In some embodiments, the polymerized lipid shell comprises a ratio of 3.5:1 of at least two lipids that differ in tail size by at least 7 carbons.

In some embodiments, the invention comprises a polymerized lipid nanoparticle comprising a polymerized lipid shell, comprising a targeting agent and a therapeutic agent, wherein the polymerized lipid nanoparticle has a potency of at least 2 fold higher than conventional liposome pegylated preparation.

In some embodiments, the polymerized lipid nanoparticle comprises a polymerized lipid shell, comprising a targeting agent and a therapeutic agent, wherein therapeutic agent to lipid molar ratio is 0.15.

In some embodiments, the polymerized lipid nanoparticle comprises a polymerizable lipid that is a C25 tail lipid. In some embodiments, the polymerized lipid nanoparticle comprises a negatively charged lipid that is a C18 tail lipid. In some embodiments, the polymerized lipid nanoparticle comprises a zwitterionically charged lipid that is a C18 tail lipid.

In some embodiments, the polymerized lipid nanoparticle is about 30 nm to about 200 nm in size.

In some embodiments, the polymerized lipid nanoparticle comprises a therapeutic agent. In some embodiments, the therapeutic agent is selected from the group comprising: antineoplastic agents, blood products, biological response modifiers; anti-fungals, hormones, vitamins, peptides, anti-tuberculars, enzymes, anti-allergic agents, anti-coagulators, circulatory drugs, metabolic potentiators, antivirals, antianginals, antibiotics, antiinflammatories, antiprotozoans, antirheumatics, narcotics, opiates, cardiac glycosides, neuromuscular blockers, sedatives, local anesthetics, general anesthetics, radioactive compounds, monoclonal antibodies, genetic material, antisense nucleic acids such as siRNA or RNAi molecules, and prodrugs.

In some embodiments, the polymerized lipid nanoparticle further comprises at least one non-polymerizable lipid selected from the group consisting of L-α-phosphatidylcholine, PE-PEG2000, 1,2-distearoyl-sn-glycero-3-phosphoethanolamine-N-[methoxy(polyethylene glycol)-2000] and PE-PEG2000-biotin.

In some embodiments, the polymerized lipid nanoparticle comprises a polymerizable lipid that is a diacetylenic lipid. In some embodiments, the polymerized lipid nanoparticle is UV treated for about 2-35 minutes after fabrication to polymerize the lipid shell. In some embodiments, the polymerized lipid nanoparticle is prepared by overnight cooling at 5-10° C. immediately after extrusion but prior to polymerization.

In some embodiments, the polymerized lipid nanoparticle comprises a targeting agent. In some embodiments, the targeting agent is selected from a group consisting of antibodies, ligands, proteins, peptides, carbohydrates, vitamins, nucleic acids and combinations thereof. In some embodiments, the targeting agent is specific to a cell surface molecule. In some embodiments, the targeting agent enhances endocytosis or cell membrane fusion.

In some embodiments, the polymerized lipid nanoparticle has a circulation half life of at least about 3 to at least about 4 hours. In some embodiments, the polymerized lipid nanoparticle is internalized into the endosome compartment of a cell after about 30 minutes.

In some embodiments, the polymerized lipid nanoparticles comprise a non-polymerizable lipid, wherein the at least one non-polymerizable lipid comprises PEG. In some embodiments, the PEG has a mass of 1000-5000 Daltons.

In some embodiments, the invention provides for a collection of polymerized lipid nanoparticle comprising the polymerized lipid nanoparticles of the invention.

In some embodiments, the invention provides for a method of treating an individual comprising: administering a polymerized lipid nanoparticle to an individual in need thereof, said polymerized lipid nanoparticle comprising a polymerized lipid shell, a targeting agent and a therapeutic agent, wherein the polymerized lipid shell comprises about 15% to about 20% of polymerizable lipid, about 1-15% of negatively charged lipid, about 20-45% of neutrally charged lipid and about 30 to 60% of zwitterionically charged lipid. In some embodiments, the non-polymerizable lipid is L-α-phosphatidylcholine, PE-PEG₂₀₀₀, 1,2-distearoyl-sn-glycero-3-phosphoethanolamine-N-[methoxy(polyethylene glycol)-2000] or PE-PEG₂₀₀₀-biotin. In some embodiments, the polymerizable lipid is a diacetylenic lipid. In some embodiments, the targeting agent is selected from a group consisting of antibodies, ligands, proteins, peptides, carbohydrates, vitamins, nucleic acids and combinations thereof. In some embodiments, the targeting agent is specific to a cell surface molecule.

In some embodiments, the polymerized lipid nanoparticle is internalized into the endosome compartment of a cell after 30 minutes of administration.

In some embodiments, the invention provides for a method of treating an individual comprising administering a polymerized lipid nanoparticle.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1: Immunoblot of osteosarcoma cell lines showing ALCAM expression.

FIG. 2: Fluorescent images of osteosarcoma cell lines showing ALCAM expression.

FIG. 3: Images of tumor samples showing ALCAM expression.

FIG. 4: Images showing targeted binding of PLNs to osteosarcoma cell lines.

FIG. 5: Fluorescent images of a time course of PLN binding to osteosarcoma cell line.

FIG. 6: Internalization of targeted PLNs in an osteosarcoma cell line at the indicated temperatures.

FIG. 7: Bar graph depicting cytotoxicity of liposomal nanoparticles containing doxorubicin.

FIG. 8: Table depicting drug loading efficiency and containment of liposomal nanoparticles.

FIG. 9: Table depicting mean IC50 of cytotoxicity by liposomal nanoparticles containing doxorubicin.

FIG. 10: Schematic of antibodies and antibody fragments.

FIG. 11: (a) and (b) Images of purified anti-CA19-9 cys-diabody and (c) elution profile.

FIG. 12: (a) Flow cytometry histograms, (b) immunofluorescence images, and (c) graph showing binding specificity of anti-CA19-9 cys-diabody.

FIG. 13: Images by microPET and microCT of xenografts at 4 and 20 hours.

FIG. 14: Schematic of conjugation reaction between polymerized lipid nanoparticle and cys-diabody.

FIG. 15: Flow cytometry histograms and images of anti-CA19-9 cys-diabody-PLN conjugate targeting in (a) MiaPaca-2 cells and (b) BxPC3 cells.

### DETAILED DESCRIPTION OF THE INVENTION

The present invention relates to the fabrication and uses of liposomal nanoparticles. In some embodiments, lipids or other components of the nanoparticles can be polymerized to form polymerized liposomal nanoparticles (PLNs), which can enhance stability and other desirable characteristic of the nanoparticles. In some embodiments, PLNs can be hybrid polymerized liposomal nanoparticles (HPLNs). In some embodiments, PLNs of the invention can be used for drug delivery, including targeted drug delivery. In some embodiments, PLNs of the invention can comprise targeting agents, therapeutic agents, contrast-enhancing agents, agents to improve cell uptake, agents that enhance or stabilize other agents in the PLNs, or combinations thereof. In some embodiments, PLNs, such as HPLNs, can be naturally fluorescent.

It should be understood that this invention is not limited to the particular methodology, protocols, and reagents, etc., described herein and as such may vary. The terminology used herein is for the purpose of describing particular embodiments only, and is not intended to limit the scope of the present invention.

Introduction

The long sought goal of being able to preferentially deliver drugs to targeted treatment areas, such as delivering anti-cancer therapies to tumors while sparing normal cells, could have a significant impact on the deficiencies of current treatment regimens. In this regard, the use of drug loadable nanoparticles as delivery vehicles appears promising. Liposomes, unilamellar vesicles composed of natural and/or synthetic lipids, have been a particularly intensively studied system. The problem of containment versus controlled release of anti-cancer agents has been a challenge for liposomal drug delivery. On the one hand, liposomes need to be formulated to allow for efficient packaging of therapeutic agents and stable containment of drug in a normal extracellular environment. On the other hand, liposomes that have localized to tumors need to be able to release their payload in order to have a therapeutic effect. This latter attribute has been particularly difficult to program into standard liposome formulations. Thus, in some embodiments, the invention provides liposomal nanoparticles that are able to release their payload to their target, e.g., cell, organ or tissue.

In some embodiments, targeting strategies involve one or more markers that are expressed on the target surface, e.g. tumor-associated molecules that are expressed at higher levels than in normal tissues. In some embodiments, nanoparticles coated with molecules (e.g. antibodies) recognizing these markers are used to bind to the target, e.g. tumor cells. In some embodiments, the markers are internalized when bound by ligands or proteins at the cell surface. Without intending to be limited by any theory, targeted nanoparticles can exploit this interaction to deliver therapeutic payloads into tumor cells through receptor-mediated endocytosis.

Liposomal nanoparticles, including PLNs, can comprise solid, liquid, or gaseous states at room temperature or at body temperature.

In some embodiments, the liposomal nanoparticle has a diameter size range that is about 3 nm-5 μm. In some embodiments, the nanoparticle has a diameter size range that is about 50 nm-5 μm. In some embodiments, the nanoparticle has a diameter size range that is about 10 nm-1.5 μm. In some embodiments, the nanoparticle has a diameter size range that is about 50 nm-120 nm. In some embodiments, the nanoparticle has a diameter size range of about 90 nm. In some embodiments, the nanoparticle has a diameter size of about 100 nm. In another embodiment, the nanoparticle has a diameter size of about 110 nm.

In some embodiments, the nanoparticles comprise one or more lipids. The term lipids includes agents exhibiting amphipathic characteristics causing them to spontaneously adopt an organized structure in water wherein the hydrophobic portion of the molecule is sequestered away from the aqueous phase. In some embodiments, the nanoparticles comprise polymerizable lipids. In some embodiments, the nanoparticles comprise one or more lipids, at least one of which is polymerizable. In some embodiments the nanoparticles comprise one or more substances inside a lipid shell. The nanoparticles may optionally also contain targeting agents, therapeutic agents, and/or other functional molecules. The nanoparticles of the invention may also include any other materials or combination thereof known to those skilled in the art as suitable for nanoparticle construction.

Lipids

In one aspect, the nanoparticles of the invention comprise one or more lipid. The lipids used may be of natural and/or synthetic origin. Such lipids include, but are not limited to, fatty acids, lysolipids, dipalmitoylphosphatidylcholine, phosphatidylcholine, phosphatidic acid, sphingomyelin, cholesterol, cholesterol hemisuccinate, tocopherol hemisuccinate, phosphatidylethanolamine, phosphatidyl-inositol, lysolipids, sphingomyelin, glycosphingolipids, glucolipids, glycolipids, sulphatides, lipids with ether and ester-linked fatty acids, diacetyl phosphate, stearylamine, distearoylphosphatidylcholine, phosphatidylserine, sphingomyelin, cardiolipin, phospholipids with short chain fatty acids of 6-8 carbons in length, synthetic phospholipids with asymmetric acyl chains (e.g., with one acyl chain of 6 carbons and another acyl chain of 12 carbons), 6-(5-cholesten-3β-yloxy)-1-thio-θ-D-galactopyranoside, digalactosyldiglyceride, 6-(5-cholesten-3β-yloxy)hexyl-6-amino-6-deoxy-1-thio-β-D-galactopyranoside, 6-(5-cholesten-3β-yloxy)hexyl-6-amino-6-deoxyl-1-thio-α-D-manno pyranoside, dibehenoyl-phosphatidylcholine, dimyristoylphosphatidylcholine, dilauroylphosphatidylcholine, and dioleoyl-phosphatidylcholine, and/or combinations thereof.

In some embodiments, the nanoparticles of the invention comprise one or more polymerizable lipids. Examples of polymerizable lipids include but are not limited to, diyne PC and diynePE, for example 1,2-bis(10,12-tricosadiynoyl)-sn-glycero-3-phosphocoline. In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of polymerizable lipids. In some embodiments, the nanoparticles of the invention comprise at least 25% of polymerizable lipids. In some embodiments, the nanoparticles of the invention comprise at least 50% of polymerizable lipids. In some embodiments, the nanoparticles of the invention comprise about 10% to about 30% of polymerizable lipids. In some embodiments, the nanoparticles of the invention comprise about 15% to about 25% of polymerizable lipids. In some embodiments, the nanoparticles of the invention comprise about 15% to about 20% of polymerizable lipids. In some embodiments, the polymerizable lipid may comprise a polymerizable group attached to a lipid molecule. The nanoparticles may also contain lipids that are not polymerizable, lipids conjugated to a functional moiety (such as a targeting agent or a therapeutic agent), and lipids with a positive, negative, or neutral charge.

In some embodiments, the nanoparticles of the invention comprise one or more neutral molecules embedded in the lipid layer. Examples of neutral molecules include, but are not limited to, cholesterol. In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of neutral molecules. In some embodiments, the nanoparticles of the invention comprise at least 10% of neutral molecules. In some embodiments, the nanoparticles of the invention comprise at least 30% of neutral molecules. In some embodiments, the nanoparticles of the invention comprise about 32% of neutral molecules. In some embodiments, the nanoparticles of the invention comprise at least 45% of neutral molecules. In some embodiments, the nanoparticles of the invention comprise about 20% to about 45% of neutral molecules.

In some embodiments, the nanoparticles of the invention comprise one or more neutral phospholipids. Examples of neutral phospholipids include, but are not limited to, hydrogenated phosphatidyl choline (HSPC), distearoyl- and diarachidoyl phosphatidylcholine (DPPC, DSPC, DAPC). In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of neutral phospholipids. In some embodiments, the nanoparticles of the invention comprise at least 10% of neutral phospholipids. In some embodiments, the nanoparticles of the invention comprise at least 30% of neutral phospholipids. In some embodiments, the nanoparticles of the invention comprise at least 45% of neutral phospholipids.

In some embodiments, the nanoparticles of the invention comprise one or more negatively charged phospholipids. Examples of negatively charged phospholipids include, but are not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG), m-Peg₂₀₀₀-DSPE and mal-Peg₂₀₀₀-DSPE. In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged phospholipids. In some embodiments, the nanoparticles of the invention comprise at least 2% of negatively charged phospholipids. In some embodiments, the nanoparticles of the invention comprise at least 5% of negatively charged phospholipids. In some embodiments, the nanoparticles of the invention comprise at least 6% of negatively charged phospholipids. In some embodiments, the nanoparticles of the invention comprise at least 10% of negatively charged phospholipids. In some embodiments, the nanoparticles of the invention comprise at least 25% of negatively charged phospholipids. In some embodiments, the nanoparticles of the invention comprise at least 30% of negatively charged phospholipids. In some embodiments, the nanoparticles of the invention comprise about 1% to about 15% of negatively charged phospholipids.

In some embodiments, the nanoparticles of the invention comprise one or more reactive phospholipids. Examples of reactive phospholipids include, but are not limited to, phosphatidyl ethanolamine derivatives coupled to a polyethyleneglycol, a biotinyl, a glutaryl, a caproyl, a maleimide, a sulfhydral, a pyridinal disulfide or a succinyl amine. In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of reactive phospholipids. In some embodiments, the nanoparticles of the invention comprise at least 2% of reactive phospholipids. In some embodiments, the nanoparticles of the invention comprise at least 4.5% of reactive phospholipids. In some embodiments, the nanoparticles of the invention comprise at least 5% of reactive phospholipids. In some embodiments, the nanoparticles of the invention comprise at least 10% of reactive phospholipids. In some embodiments, the nanoparticles of the invention comprise at least 25% of reactive phospholipids. In some embodiments, the nanoparticles of the invention comprise at least 30% of reactive phospholipids.

In some embodiments, the nanoparticles of the invention comprise one or more zwitterionic lipids, e.g., hydrogenated soy PC. In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of zwitterionic lipids. In some embodiments, the nanoparticles of the invention comprise about 30% to about 60% of zwitterionic lipids. In some embodiments, the nanoparticles of the invention comprise at least 20% of zwitterionic lipids. In some embodiments, the nanoparticles of the invention comprise at least 35% of zwitterionic lipids. In some embodiments, the nanoparticles of the invention comprise at least 45% of zwitterionic lipids. In some embodiments, the nanoparticles of the invention comprise at least 50% of zwitterionic lipids. In some embodiments, the nanoparticles of the invention comprise about 47% of zwitterionic lipids.

In some embodiments, the nanoparticles of the invention comprise about 30% zwitterionic lipids, about 2% negatively charged lipids, and the rest uncharged lipids. In some embodiments, the nanoparticles of the invention comprise about 30% zwitterionic lipids, about 3% negatively charged lipids, and the rest uncharged lipids. In some embodiments, the nanoparticles of the invention comprise about 30% zwitterionic lipids, about 4% negatively charged lipids, and the rest uncharged lipids. In some embodiments, the nanoparticles of the invention comprise about 30% zwitterionic lipids, about 5% negatively charged lipids, and the rest uncharged lipids. In some embodiments, the nanoparticles of the invention comprise about 30% zwitterionic lipids, about 6% negatively charged lipids, and the rest uncharged lipids. In some embodiments, the nanoparticles of the invention comprise about 30% zwitterionic lipids, about 7% negatively charged lipids, and the rest uncharged lipids. In some embodiments, the nanoparticles of the invention comprise about 30% zwitterionic lipids, about 8% negatively charged lipids, and the rest uncharged lipids. In some embodiments, the nanoparticles of the invention comprise about 30% zwitterionic lipids, about 9% negatively charged lipids, and the rest uncharged lipids. In some embodiments, the nanoparticles of the invention comprise about 30% zwitterionic lipids, about 10% negatively charged lipids, and the rest uncharged lipids.

In some embodiments, the nanoparticles of the invention comprise about 35% zwitterionic lipids, about 2% negatively charged lipids, and the rest uncharged lipids. In some embodiments, the nanoparticles of the invention comprise about 35% zwitterionic lipids, about 3% negatively charged lipids, and the rest uncharged lipids. In some embodiments, the nanoparticles of the invention comprise about 35% zwitterionic lipids, about 4% negatively charged lipids, and the rest uncharged lipids. In some embodiments, the nanoparticles of the invention comprise about 35% zwitterionic lipids, about 5% negatively charged lipids, and the rest uncharged lipids. In some embodiments, the nanoparticles of the invention comprise about 35% zwitterionic lipids, about 6% negatively charged lipids, and the rest uncharged lipids. In some embodiments, the nanoparticles of the invention comprise about 35% zwitterionic lipids, about 7% negatively charged lipids, and the rest uncharged lipids. In some embodiments, the nanoparticles of the invention comprise about 35% zwitterionic lipids, about 8% negatively charged lipids, and the rest uncharged lipids. In some embodiments, the nanoparticles of the invention comprise about 35% zwitterionic lipids, about 9% negatively charged lipids, and the rest uncharged lipids. In some embodiments, the nanoparticles of the invention comprise about 35% zwitterionic lipids, about 10% negatively charged lipids, and the rest uncharged lipids.

In some embodiments, the nanoparticles of the invention comprise about 40% zwitterionic lipids, about 2% negatively charged lipids, and the rest uncharged lipids. In some embodiments, the nanoparticles of the invention comprise about 40% zwitterionic lipids, about 3% negatively charged lipids, and the rest uncharged lipids. In some embodiments, the nanoparticles of the invention comprise about 30% zwitterionic lipids, about 4% negatively charged lipids, and the rest uncharged lipids. In some embodiments, the nanoparticles of the invention comprise about 40% zwitterionic lipids, about 5% negatively charged lipids, and the rest uncharged lipids. In some embodiments, the nanoparticles of the invention comprise about 40% zwitterionic lipids, about 6% negatively charged lipids, and the rest uncharged lipids. In some embodiments, the nanoparticles of the invention comprise about 40% zwitterionic lipids, about 7% negatively charged lipids, and the rest uncharged lipids. In some embodiments, the nanoparticles of the invention comprise about 40% zwitterionic lipids, about 8% negatively charged lipids, and the rest uncharged lipids. In some embodiments, the nanoparticles of the invention comprise about 40% zwitterionic lipids, about 9% negatively charged lipids, and the rest uncharged lipids. In some embodiments, the nanoparticles of the invention comprise about 40% zwitterionic lipids, about 10% negatively charged lipids, and the rest uncharged lipids.

In some embodiments, the nanoparticles of the invention comprise about 45% zwitterionic lipids, about 2% negatively charged lipids, and the rest uncharged lipids. In some embodiments, the nanoparticles of the invention comprise about 45% zwitterionic lipids, about 3% negatively charged lipids, and the rest uncharged lipids. In some embodiments, the nanoparticles of the invention comprise about 45% zwitterionic lipids, about 4% negatively charged lipids, and the rest uncharged lipids. In some embodiments, the nanoparticles of the invention comprise about 45% zwitterionic lipids, about 5% negatively charged lipids, and the rest uncharged lipids. In some embodiments, the nanoparticles of the invention comprise about 45% zwitterionic lipids, about 6% negatively charged lipids, and the rest uncharged lipids. In some embodiments, the nanoparticles of the invention comprise about 45% zwitterionic lipids, about 7% negatively charged lipids, and the rest uncharged lipids. In some embodiments, the nanoparticles of the invention comprise about 45% zwitterionic lipids, about 8% negatively charged lipids, and the rest uncharged lipids. In some embodiments, the nanoparticles of the invention comprise about 45% zwitterionic lipids, about 9% negatively charged lipids, and the rest uncharged lipids. In some embodiments, the nanoparticles of the invention comprise about 45% zwitterionic lipids, about 10% negatively charged lipids, and the rest uncharged lipids.

In some embodiments, the nanoparticles of the invention comprise about 50% zwitterionic lipids, about 2% negatively charged lipids, and the rest uncharged lipids. In some embodiments, the nanoparticles of the invention comprise about 50% zwitterionic lipids, about 3% negatively charged lipids, and the rest uncharged lipids. In some embodiments, the nanoparticles of the invention comprise about 50% zwitterionic lipids, about 4% negatively charged lipids, and the rest uncharged lipids. In some embodiments, the nanoparticles of the invention comprise about 50% zwitterionic lipids, about 5% negatively charged lipids, and the rest uncharged lipids. In some embodiments, the nanoparticles of the invention comprise about 50% zwitterionic lipids, about 6% negatively charged lipids, and the rest uncharged lipids. In some embodiments, the nanoparticles of the invention comprise about 50% zwitterionic lipids, about 7% negatively charged lipids, and the rest uncharged lipids. In some embodiments, the nanoparticles of the invention comprise about 50% zwitterionic lipids, about 8% negatively charged lipids, and the rest uncharged lipids. In some embodiments, the nanoparticles of the invention comprise about 50% zwitterionic lipids, about 9% negatively charged lipids, and the rest uncharged lipids. In some embodiments, the nanoparticles of the invention comprise about 50% zwitterionic lipids, about 10% negatively charged lipids, and the rest uncharged lipids.

In some embodiments, the nanoparticles of the invention comprise about 55% zwitterionic lipids, about 2% negatively charged lipids, and the rest uncharged lipids. In some embodiments, the nanoparticles of the invention comprise about 55% zwitterionic lipids, about 3% negatively charged lipids, and the rest uncharged lipids. In some embodiments, the nanoparticles of the invention comprise about 55% zwitterionic lipids, about 4% negatively charged lipids, and the rest uncharged lipids. In some embodiments, the nanoparticles of the invention comprise about 55% zwitterionic lipids, about 5% negatively charged lipids, and the rest uncharged lipids. In some embodiments, the nanoparticles of the invention comprise about 55% zwitterionic lipids, about 6% negatively charged lipids, and the rest uncharged lipids. In some embodiments, the nanoparticles of the invention comprise about 55% zwitterionic lipids, about 7% negatively charged lipids, and the rest uncharged lipids. In some embodiments, the nanoparticles of the invention comprise about 55% zwitterionic lipids, about 8% negatively charged lipids, and the rest uncharged lipids. In some embodiments, the nanoparticles of the invention comprise about 55% zwitterionic lipids, about 9% negatively charged lipids, and the rest uncharged lipids. In some embodiments, the nanoparticles of the invention comprise about 55% zwitterionic lipids, about 10% negatively charged lipids, and the rest uncharged lipids.

In some embodiments, any percentage of the uncharged lipids can be polymerizable lipids. In some embodiments, any percentage of the negatively charged lipids can comprise a linking group, such as a maleimide.

In some embodiments, the nanoparticles of the invention comprise one or more lipids and phospholipids such as soy lecithin, partially refined lecithin, hydrogenated phospholipids, lysophosphate, phosphatidylcholine, phosphatidylethanolamine, phosphatidylserine, phosphatidylinositol, cardiolipin, sphingolipids, gangliosides, cerebrosides, ceramides, other esters analogue of phosphatidylcholine (PAF, lysoPAF). In some embodiments, the nanoparticles of the invention comprise one or more synthetic phospholipids such as L-α-lecithin (dilauroylphosphatidylcholine, dipalmitoylphosphatidylcholine, dilinoloylphosphatidylcholine, distearoylphosphatidylcholine, diarachidoylphosphatidylcholine); phosphatidylethanolamine derivatives, such as 1,2-diacyl-sn-glycero-3-phosphoethanolamine, 1-acyl-2-acyl-sn-glycero-3-phosphoethanolamine, dinitrophenyl- and dinitrophenylamino caproylphosphatidylethanolamine, 1,2-diacyl-sn-glycero-3-phosphoethanolamine-N-polyethylene glycol (PEG-PE), N-biotinyl-PE, N-caproylamine PE, N-dodecylamine-PE, N-MPB-PE, N-PDD-PE, N-succinyl-PE, N-glutaryl-PE; di-acetylenic lipids; phosphatidic acids (1,2-diacyl-sn-glycero-3-phosphate salt, 1-acyl-2-acyl-sn-glycero-3-phosphate sodium salt; phosphatidylserine such as 1,2-diacyl-snglycero-3-[phospho-L-serine]sodium salt, 1-acyl-2-acyl-sn-glycero-3-[phospho-L-serine]sodium salt, lysophosphatidic acid; cationic lipids such as 1,2-diacyl-3-trimethylammoniumpropane (TAP), 1,2-diacyl-3-dimethylammoniumpropane (DAP), N-[1-(2,3-dioleoyloxy) propyl-N,N′,N″-trimethylammonium chloride (DOTMA).

In some embodiments, the nanoparticles of the invention comprise one or more lipids suitable for click chemistry, such as those containing azide and alkyne groups. In some embodiments, the nanoparticles of the invention comprise one or more phospholipids with multivarious headgroups such as phosphatidylethanol, phosphatidylpropanol and phosphatidylbutanol, phosphatidylethanolamine-N-monomethyl, 1,2-disteraoyl(dibromo)-sn-glycero-3-phosphocoline. In some embodiments, the nanoparticles of the invention comprise one or more phospholipids with partially or fully fluorinated cholesterol or cholesterol derivatives can be used in place of an uncharged lipid, as generally known to a person skilled in the art.

The surface of a nanoparticles may also be modified with a polymer, such as, for example, with polyethylene glycol (PEG), using procedures readily apparent to those skilled in the art. Lipids may contain functional surface groups for attachment to a metal, which provides for the chelation of radioactive isotopes or other materials that serve as the therapeutic entity. Any species of lipid may be used, with the sole proviso that the lipid or combination of lipids and associated materials incorporated within the lipid matrix should form a′nanoparticle under physiologically relevant conditions. As one skilled in the art will recognize, the composition of the nanoparticle may be altered to modulate the biodistribution and clearance properties of the resulting nanoparticles.

Other useful lipids or combinations thereof apparent to those skilled in the art which are in keeping with the spirit of the present invention are also encompassed by the present invention. For example, carbohydrates bearing lipids may be employed for in vivo targeting as described in U.S. Pat. No. 4,310,505.

In some embodiments, the nanoparticles of the invention comprise one or more polymerizable lipid. Polymerizable lipids that can be used in the present invention include those described in U.S. Pat. Nos. 5,512,294 and 6,132,764, and US publication No. 2010/0111840, incorporated by reference herein in their entirety.

In some embodiments, the hydrophobic tail groups of polymerizable lipids are derivatized with polymerizable groups, such as diacetylene groups, which irreversibly cross-link, or polymerize, when exposed to ultraviolet light or other radical anionic or cationic initiating species, while maintaining the distribution of functional groups at the surface of the nanoparticle. The resulting polymerized nanoparticle is stabilized against fusion with cell membranes or other nanoparticles and stabilized towards enzymatic degradation. The size of the polymerized nanoparticles can be controlled by the method described herein, but also by other methods known to those skilled in the art, for example, by extrusion.

Polymerized nanoparticles may be comprised of polymerizable lipids, but may also comprise saturated and non-alkyne, unsaturated lipids. The polymerized nanoparticles can be a mixture of lipids which provide different functional groups on the hydrophilic exposed surface. For example, some hydrophilic head groups can have functional surface groups, for example, biotin, amines, cyano, carboxylic acids, isothiocyanates, thiols, disulfides, α-halocarbonyl compounds, α,β-unsaturated carbonyl compounds and alkyl hydrazines. These groups can be used for attachment of targeting agents, such as antibodies, ligands, proteins, peptides, carbohydrates, vitamins, nucleic acids or combinations thereof for specific targeting and attachment to desired cell surface molecules, and for attachment of therapeutic agents, such as drugs, nucleic acids encoding genes with therapeutic effect or radioactive isotopes. Other head groups may have an attached or encapsulated therapeutic agent, such as, for example, antibodies, hormones and drugs for interaction with a biological site at or near the specific biological molecule to which the polymerized nanoparticle attaches. Other hydrophilic head groups can have a functional surface group of diethylenetriamine pentaacetic acid, ethylenedinitrile tetraacetic acid, tetraazocyclododecane-1,4,7,10-tetraacetic acid (DOTA), porphoryin chelate and cyclohexane-1,2,-diamino-N,N′-diacetate, as well as derivatives of these compounds, for attachment to a metal, which provides for the chelation of radioactive isotopes or other materials that serve as the therapeutic entity. Examples of lipids with chelating head groups are provided in U.S. Pat. No. 5,512,294, incorporated by reference herein in its entirety.

In some embodiments, nanoparticles are hybrid polymerizable liposomal nanoparticles. Hybrid PLNs can comprise any of the lipids described herein for liposomal nanoparticles, including charged lipids, uncharged lipids, cholesterols, and any combinations thereof. Charged lipids include positively charged lipids; negatively charged lipids such as of m-Peg2000-DSPE and mal-Peg2000-DSPE; and zwitterionically charged lipids, such as L-α-phosphatidylcholine hydrogenated soy (hydro soy PC). In some embodiments, HPLNs comprise the polymerizable, uncharged lipid N-(5′-hydroxy-3′-oxypentyl)-10-12-pentacosadiynamide (h-Peg₁-PCDA). In a preferred embodiment, HPLNs comprise cholesterol, h-Peg₁-PCDA, hydro soy PC, of m-Peg2000-DSPE and mal-Peg2000-DSPE.

The component lipids of polymerized nanoparticles can be purified and characterized individually using standard, known techniques and then combined in controlled fashion to produce the final particle. The polymerized nanoparticles can be constructed to mimic native cell membranes or present functionality, such as ethylene glycol derivatives, that can reduce their potential immunogenicity. Additionally, the polymerized nanoparticles have a well-defined structure that can be characterized by known physical techniques such as transmission electron microscopy and atomic force microscopy.

In some embodiments, the nanoparticles can be formed from lipid solutions by any suitable method known in the art.

Targeting Agents

In some embodiments, the nanoparticles of the invention comprise a targeting agent. The term targeting agent includes a molecule, macromolecule, or molecular assembly which binds specifically to a biological target. Any biologically compatible, natural or artificial molecule may be utilized as a targeting agent. Examples of targeting agents include, but are not limited to, amphetamines, barbiturates, sulfonamides, monoamine oxydase inhibitor substrates, antibodies (including antibody fragments and other antibody-derived molecules which retain specific binding, such as Fab, F(ab′)2, Fv, diabodies and scFv derived from antibodies); receptor-binding ligands, such as hormones or other molecules that bind specifically to a receptor; cytokines, which are polypeptides that affect cell function and modulate interactions between cells associated with immune, inflammatory or hematopoietic responses; molecules that bind to enzymes, such as enzyme inhibitors; ligands specific to cellular membranes; enzymes, lipids, nucleic acid ligands or aptamers, antihypertensive agents, neurotransmitters, amino acids, oligopeptides, radio-sensitizers, steroids (e.g. estrogen and estradiol), mono- and carbohydrates (such as glucose derivatives), fatty acids, muscarine receptors and substrates (such as 3-quinuclidinyle benzilate), dopamine receptors and substrates (such as spiperone), one or more members of a specific binding interaction such as biotin or iminobiotin and avidin or streptavidin and peptides, and proteins capable of binding specific receptors.

In some embodiments, targeting agents are molecules which specifically bind to receptors or antigens found on vascular cells. In some embodiments, targeting agents are molecules which specifically bind to receptors, antigens or markers found on cells of angiogenic neovasculature or receptors, antigens or markers associated with tumor vasculature. The receptors, antigens or markers associated with tumor vasculature can be expressed on cells of vessels which penetrate or are located within the tumor, or which are confined to the inner or outer periphery of the tumor. In one embodiment, the invention takes advantage of pre-existing or induced leakage from the tumor vascular bed; in this embodiment, tumor cell antigens can also be directly targeted with agents that pass from the circulation into the tumor interstitial volume.

In some embodiments, the targeting agents target endothelial receptors, tissue or other targets accessible through a body fluid or receptors or other targets upregulated in a tissue or cell adjacent to or in a bodily fluid. Targeting agents attached to the polymerized nanoparticles, or linking carriers of the invention include, but are not limited to, small molecule ligands, such as carbohydrates, and compounds such as those disclosed in U.S. Pat. No. 5,792,783 (small molecule ligands are defined herein as organic molecules with a molecular weight of about 1000 daltons or less, which serve as ligands for a vascular target or vascular cell marker); proteins, such as antibodies and growth factors; peptides, such as RGD-containing peptides (e.g. those described in U.S. Pat. No. 5,866,540), bombesin or gastrin-releasing peptide, peptides selected by phage-display techniques such as those described in U.S. Pat. No. 5,403,484, and peptides designed de novo to be complementary to tumor-expressed receptors; antigenic determinants; or other receptor targeting groups.

These head groups can be used to control the biodistribution, non-specific adhesion, and blood pool half-life of the polymerized nanoparticles. For example, β-D-lactose has been attached on the surface to target the asialoglycoprotein (ASG) found in liver cells which are in contact with the circulating blood pool. Glycolipids can be derivatized for use as targeting agents by converting the commercially available lipid (DAGPE) or the PEG-PDA amine into its isocyanate, followed by treatment with triethylene glycol diamine spacer to produce the amine terminated thiocarbamate lipid, which by treatment with the para-isothiocyanophenyl glycoside of the carbohydrate ligand produces the desired targeting glycolipids. This synthesis provides a water-soluble flexible spacer molecule spaced between the lipids that form the internal structure or core of the nanoparticle and the ligand that binds to cell surface receptors, allowing the ligand to be readily accessible to the protein receptors on the cell surfaces. The carbohydrate ligands can be derived from reducing sugars or glycosides, such as para-nitrophenyl glycosides, a wide range of which are commercially available or easily constructed using chemical or enzymatic methods.

In some embodiments, the targeting agent targets the nanoparticles to a cell surface. Delivery of the therapeutic or imaging agent can occur through endocytosis of the nanoparticles or through binding to the outside of the cell. Such deliveries are known in the art. See, for example, Mastrobattista, et al., Immunoliposomes for the Targeted Delivery of Antitumor Drugs, Adv. Drug Del. Rev. (1999) 40:103-27.

In some embodiments, the targeting agent is attached to a stabilizing entity. In one embodiment, the attachment is by covalent means. In another embodiment, the attachment is by non-covalent means. For example, antibody targeting agents may be attached by a biotin-avidin biotinylated antibody sandwich, to allow a variety of commercially available biotinylated antibodies to be used on the coated polymerized nanoparticle. Specific vasculature targeting agents of use in the invention include (but are not limited to) anti-VCAM-1 antibodies (VCAM=vascular cell adhesion molecule); anti-ICAM-1 antibodies (ICAM=intercellular adhesion molecule); anti-integrin antibodies (e.g., antibodies directed against αᵥβ₃ integrins such as LM609, described in International Patent Application WO 89/05155 and Cheresh et al. J. Biol. Chem. 262:17703-11 (1987), and Vitaxin, described in International Patent Application WO 9833919 and in Wu et al., Proc. Natl. Acad. Sci. USA 95(11):6037-42 (1998); and antibodies directed against P- and E-selectins, pleiotropin and endosialin, endoglin, VEGF receptors, PDGF receptors, EGF receptors, FGF receptors, MMPs, and prostate specific membrane antigen (PSMA). Additional targets are described by E. Ruoslahti in Nature Reviews: Cancer, 2, 83-90 (2002).

In one embodiment of the invention, the targeted agent is combined with an agent targeted directly towards tumor cells. This embodiment takes advantage of the fact that the neovasculature surrounding tumors is often highly permeable or “leaky,” allowing direct passage of materials from the bloodstream into the interstitial space surrounding the tumor. Alternatively, the targeted agent itself can induce permeability in the tumor vasculature. For example, when the agent carries a radioactive therapeutic agent, upon binding to the vascular tissue and irradiating that tissue, cell death of the vascular epithelium will follow and the integrity of the vasculature will be compromised.

In some embodiments, the targeting agents can be attached to the nanoparticles using any feasible method known in the art such as carbodiimide, maleimide, disulfide, or biotin-streptavidin coupling.

Therapeutic Agents

In some embodiments, a therapeutic agent may be incorporated into the nanoparticles. A variety of drugs and other bioactive compounds may be incorporated into the nanoparticles, including antineoplastic agents, blood products, biological response modifiers; anti-fungals, hormones, vitamins, peptides, anti-tuberculars, enzymes, anti-allergic agents, anti-coagulators, circulatory drugs, metabolic potentiators, antivirals, antianginals, antibiotics, antiinflammatories, antiprotozoans, antirheumatics, narcotics, opiates, cardiac glycosides, neuromuscular blockers, sedatives, local anesthetics, general anesthetics, radioactive compounds, monoclonal antibodies, genetic material, antisense nucleic acids such as siRNA or RNAi molecules, and prodrugs.

In some embodiments, some of the bioactive compounds that may be incorporated into the nanoparticles include genetic material such as nucleic acids, RNA, and DNA, of either natural or synthetic origin, including recombinant RNA and DNA and antisense RNA and DNA such as siRNAs or RNAi molecules, genes carried on expression vectors such as plasmids, phagemids, cosmids, yeast artificial chromosomes (YACs), and defective or “helper” viruses, antigene nucleic acids, both single and double stranded RNA and DNA and analogs thereof; hormone products such as vasopressin, oxytocin, progestins, estrogens and antiestrogens and their derivatives, glucagon, and thyroid agents such as iodine products and anti-thyroid agents; biological response modifiers such as muramyldipeptide, muramyltripeptide, microbial cell wall components, lymphokines (e.g., bacterial endotoxins such as lipopolysaccharide, macrophage activation factor), subunits of bacteria (such as Mycobacteria, Corynebacteria), the synthetic dipeptide N-acctyl-muramyl-L-alanyl-Disoglutamine; cardiovascular products such as chelating agents and mercurial diuretics and cardiac glycosides; blood products such as parenteral iron, hemin, hematoporphyrins and their derivatives; respiratory products such as xanthine derivatives (theophylline & aminophylline); anti-infectives such as aminoglycosides, antifungals (amphotericin, ketoconazole, nystatin, griseofulvin, flucytosine (5-fc), miconazole, amphotericin B, ricin, and 13-lactam antibiotics (e.g., sulfazecin)), antibiotics such as penicillins, actinomycin and cephalosporins, antiviral agents such as Zidovudine, Ribavirin, Amantadine, Vidarabine, and Acyclovir, anti-helmintics, antimalarials, and antituberculous drugs; biologicals such as immune serums, antitoxins and antivenins, rabies prophylaxis products, bacterial vaccines, viral vaccines, toxoids; antineoplastics such as nitrosureas, hydroxyurea, procarbazine, Dacarbazine, mitotane, nitrogen mustards, antimetabolites (fluorouracil), platinum compounds (e.g., spiroplatin, cisplatin, and carboplatin), methotrexate, adriamycin, taxol, mitomycin, ansamitocin, bleomycin, cytosine arabinoside, arabinosyl adenine, mercaptopolylysine, vincristine, busulfan, chlorambucil, melphalan (e.g., PAM, L-PAM or phenylalanine mustard), mercaptopurine, dactinomycin (actinomycin D), daunorubicin hydrochloride, doxorubicin hydrochloride, mitomycin, plicamycin (mithramycin), aminoglutethimide, estramustine phosphate sodium, flutamide, leuprolide acetate, megestrol acetate, tamoxifen citrate, testolactone, trilostane, amsacrine (m-AMSA), asparaginase (L-asparaginase) Erwina asparaginase, etoposide (VP-16), teniposide (VM-26), vinblastine sulfate (VLB), vincristine sulfate, bleomycin sulfate, arabinosyl, and alkylated derivatives of metallocene dihalides; mitotic inhibitors such as etoposide and the vinca alkaloids, radiopharmaceuticals such as radioactive iodine and phosphorus products; as well as interferons (Interferon α-2a and α-2b), Asparaginase and cyclosporin.

The bioactives may be incorporated into nanoparticles singly or in combination with each other or with additional substances aimed to increase bioactive efficacy, such as adjuvants. Bioactives may be attached (covalently, such as by ester, substituted ester, anhydride, carbohydrate, polylactide, or substituted anhydride bonds, or non-covalently, such as by streptavidin linkages or ionic binding) to the surface of the nanoparticle directly to the lipids or to a moiety conjugated to the lipids, incorporated directly into the lipid membrane, or included in the interior of the nanoparticle. In a preferred embodiment, a targeting agent can be chemically coupled to the surface of a PLN through a maleimide linkage.

In some embodiments, the liposomal nanoparticles may include targeting agents to selectively concentrate the nanoparticles to a particular region for imaging or therapeutic treatment. The targeted method is particularly suitable for diagnostic imaging to determine locations of tumors or atherosclerotic plaques. Targeting also enhances local administration of toxic substances which, if not targeted, could (and would) otherwise cause significant secondary effects to other organs; such drugs include for instance Amphotericin B or NSAID's or drugs whose administration is required over prolonged periods such as Dexamethasone, insulin, vitamin E, etc. The method is also suitable for administration of thrombolytic agents such as urokinase or streptokinase, or antitumoral compounds such as Taxol, etc.

Prodrugs and otherwise non-active agents may also be incorporated into nanoparticles with an activator, such as a protease that removes an inactivating peptide, such that the agent and the activator are separated until the nanoparticle is dissolved. Alternatively, the agent and the activator may be incorporated into different populations of nanoparticles and targeted to the same location so that the agent is selectively activated only at the target site.

Contrasting Agents

In some embodiments, the nanoparticles described herein may also contain substances to enhance imaging, e.g., for diagnostics or to visualize treatment during drug delivery. Any suitable contrasting agent known in the art can be incorporated into the nanoparticles. These can include paramagnetic substances, such as paramagnetic ions such as Mn⁺², Gd⁺², and Fe⁺³, to be used as susceptibility contrast agents for magnetic resonance imaging. Nanoparticles may contain radioopaque metal ions, such as iodine, barium, bromine, or tungsten, for use as x-ray contrast agents.

Carriers

In some embodiments, the nanoparticles comprise a linking carrier. The term linking carrier includes entities that serve to link agents, e.g., targeting agents and/or therapeutic agents, to the nanoparticles. In some embodiments, the linking carrier serves to link a therapeutic agent and the targeting agent. In some embodiments, the linking carrier confers additional advantageous properties to the nanoparticles. Examples of these additional advantages include, but are not limited to: 1) multivalency, which is defined as the ability to attach either i) multiple therapeutic agents and/or targeting agents to the nanoparticles (e.g., several units of the same therapeutic agent, or one or more units of different therapeutic entities), which increases the effective “payload” of the therapeutic entity delivered to the targeted site; ii) multiple targeting agents to nanoparticle (e.g., one or more units of the same or different therapeutic agents); and 2) improved circulation lifetimes, which can include tuning the size of the particle to achieve a specific rate of clearance by the reticuloendothelial system.

In some embodiments, the linking carriers are biocompatible polymers (such as dextran) or macromolecular assemblies of biocompatible components (such as PLNs). Examples of linking carriers include, but are not limited to, liposomal nanoparticles, polymerized liposomal nanoparticles, other lipid vesicles, dendrimers, polyethylene glycol assemblies, capped polylysines, poly(hydroxybutyric acid), dextrans, and coated polymers. A preferred linking carrier is a polymerized liposomal nanoparticle. Another preferred linking carrier is a dendrimer.

The linking carrier can be coupled to the targeting agent and/or the therapeutic agent by a variety of methods, depending on the specific chemistry involved. The coupling can be covalent or non-covalent. A variety of methods suitable for coupling of the targeting entity and the therapeutic entity to the linking carrier can be found in Hermanson, “Bioconjugate Techniques”, Academic Press: New York, 1996; and in “Chemistry of Protein Conjugation and Cross-linking” by S. S. Wong, CRC Press, 1993. Specific coupling methods include, but are not limited to, the use of bifunctional linkers, carbodiimide condensation, disulfide bond formation, and use of a specific binding pair where one member of the pair is on the linking carrier and another member of the pair is on the therapeutic or targeting entity, e.g. a biotin-avidin interaction.

Stabilizing Entities

In some embodiments, the liposomal nanoparticles contain a stabilizing entity. As used herein, “stabilizing” refers to the ability to impart additional advantages to the nanoparticles, for example, physical stability, i.e., longer half-life, colloidal stability, and/or capacity for multivalency; that is, increased payload capacity due to numerous sites for attachment of targeting agents. Stabilizing entities include macromolecules or polymers, which may optionally contain chemical functionality for the association of the stabilizing entity to the surface of the nanoparticle, and/or for subsequent association of therapeutic agents and/or targeting agents. The polymer should be biocompatible with aqueous solutions. Polymers useful to stabilize the nanoparticles of the present invention may be of natural, semi-synthetic (modified natural) or synthetic origin. A number of stabilizing entities which may be employed in the present invention are available, including xanthan gum, acacia, agar, agarose, alginic acid, alginate, sodium alginate, carrageenan, gelatin, guar gum, tragacanth, locust bean, bassorin, karaya, gum arabic, pectin, casein, bentonite, unpurified bentonite, purified bentonite, bentonite magma, and colloidal bentonite.

Other natural polymers include naturally occurring polysaccharides, such as, for example, arabinans, fructans, furans, galactans, galacturonans, glucans, mannans, xylans (such as, for example, inulin), levan, fucoidan, carrageenan, galatocarolose, pectic acid, pectins, including amylose, pullulan, glycogen, amylopectin, cellulose, dextran, dextrose, dextrin, glucose, polyglucose, polydextrose, pustulan, chitin, agarose, keratin, chondroitin, dermatan, hyaluronic acid, alginic acid, xanthin gum, starch and various other natural homopolymer or heteropolymers, such as those containing one or more of the following aldoses, ketoses, acids or amines: erythrose, threose, ribose, arabinose, xylose, lyxose, allose, altrose, glucose, dextrose, mannose, gulose, idose, galactose, talose, erythrulose, ribulose, xylulose, psicose, fructose, sorbose, tagatose, mannitol, sorbitol, lactose, sucrose, trehalose, maltose, cellobiose, glycine, serine, threonine, cysteine, tyrosine, asparagine, glutamine, aspartic acid, glutamic acid, lysine, arginine, histidine, glucuronic acid, gluconic acid, glucaric acid, galacturonic acid, mannuronic acid, glucosamine, galactosamine, and neuraminic acid, and naturally occurring derivatives thereof. Other suitable polymers include protein, such as albumin, polyalginates, and polylactide-glycolide copolymers, cellulose, cellulose (microcrystalline), methylcellulose, hydroxyethylcellulose, hydroxypropylcellulose, hydroxypropylmethylcellulose, carboxymethylcellulose, and calcium carboxymethylcellulose.

Exemplary semi-synthetic polymers include carboxymethylcellulose, sodium carboxymethylcellulose, carboxymethylcellulose sodium 12, hydroxymethylcellulose, hydroxypropylmethylcellulose, methylcellulose, and methoxycellulose. Other semi-synthetic polymers suitable for use in the present invention include carboxydextran, aminodextran, dextran aldehyde, chitosan, and carboxymethyl chitosan.

Exemplary synthetic polymers include poly(ethylene imine) and derivatives, polyphosphazenes, hydroxyapatites, fluoroapatite polymers, polyethylenes (such as, for example, polyethylene glycol, the class of compounds referred to as Pluronics®, commercially available from BASF, (Parsippany, N.J.), polyoxyethylene, and polyethylene terephthlate), polypropylenes (such as, for example, polypropylene glycol), polyurethanes (such as, for example, polyvinyl alcohol (PVA), polyvinyl chloride and polyvinylpyrrolidone), polyamides including nylon, polystyrene, polylactic acids, fluorinated hydrocarbon polymers, fluorinated carbon polymers (such as, for example, polytetrafluoroethylene), acrylate, methacrylate, and polymethylmethacrylate, and derivatives thereof, polysorbate, carbomer 934P, magnesium aluminum silicate, aluminum monostearate, polyethylene oxide, polyvinylalcohol, povidone, polyethylene glycol, and propylene glycol. Methods for the preparation of nanoparticles which employ polymers to stabilize nanoparticle compositions will be readily apparent to one skilled in the art, in view of the present disclosure, when coupled with information known in the art, such as that described and referred to in Unger, U.S. Pat. No. 5,205,290, the disclosure of which is herein incorporated by reference in its entirety.

In some embodiments, the stabilizing entity is dextran. In some embodiments, the stabilizing entity is a modified dextran, such as amino dextran. In a further preferred embodiment, the stabilizing entity is poly(ethylene imine) (PEI). Without being bound by theory, it is believed that dextran may increase circulation times of nanoparticles in a manner similar to PEG. Additionally, each polymer chain (i.e. aminodextran or succinylated aminodextran) contains numerous sites for attachment of targeting agents, providing the ability to increase the payload of the entire lipid construct. This ability to increase the payload differentiates the stabilizing agents of the present invention from PEG. For PEG there is only one site of attachment, thus the targeting agent loading capacity for PEG (with a single site for attachment per chain) is limited relative to a polymer system with multiple sites for attachment.

In some embodiments, the following polymers and their derivatives are used poly(galacturonic acid), poly(L-glutamic acid), poly(L-glutamic acid-L-tyrosine), poly[R)-3-hydroxybutyric acid], poly(inosinic acid potassium salt), poly(L-lysine), poly(acrylic acid), poly(ethanolsulfonic acid sodium salt), poly(methylhydrosiloxane), polyvinyl alcohol), poly(vinylpolypyrrolidone), poly(vinylpyrrolidone), poly(glycolide), poly(lactide), poly(lactide-co-glycolide), and hyaluronic acid. In other preferred embodiments, copolymers including a monomer having at least one reactive site, and preferably multiple reactive sites, for the attachment of the copolymer to the nanoparticle or other molecule.

In some embodiments, the polymer may act as a hetero- or homobifunctional linking agent for the attachment of targeting agents, therapeutic entities, proteins or chelators such as DTPA and its derivatives.

In some embodiments, the stabilizing entity is associated with the nanoparticle by covalent means. In another embodiment, the stabilizing entity is associated with the nanoparticle by non-covalent means. Covalent means for attaching the targeting entity to the nanoparticles are known in the art and described in the US publication 2010/0111840 entitled Stabilized Therapeutic and Imaging Agents, incorporated by reference herein in its entirety.

Noncovalent means for attaching the targeting entity with the nanoparticle include but are not limited to attachment via ionic, hydrogen-bonding interactions, including those mediated by water molecules or other solvents, hydrophobic interactions, or any combination of these.

In some embodiments, the stabilizing agent forms a coating on the nanoparticle.

In some embodiments, the liposomal nanoparticles of the invention may also be linked to functional agents, such as poly(ethylene glycol) (PEG), that otherwise modify nanoparticle properties, such as aggregation tendencies, binding by opsonizing plasma proteins, uptake by cells, and stability in the bloodstream.

Other Agents

In some embodiments, liposomal nanoparticles of the invention can comprise other bioactive agents, pharmaceutical carriers, or other substances that modulate properties of the nanoparticles, including but not limited to nanoparticle targeting, stability, detectability or endocytosis; and activity, stability or specificity of therapeutic agents. In some embodiments, liposomal nanoparticles of the invention can comprise markers that enhance cellular uptake or endocytosis, preferably by its target cells or tissue. In some embodiments, the nanoparticles can comprise membrane fusion proteins to enhance nanoparticle fusion with cellular membranes. Examples of such membrane fusion proteins include but are not limited to Gp41, FAST, SNAP, and SNARE proteins, such as VAMP. In some embodiments, the nanoparticles can comprise ligands for cell surface receptors that, when activated, trigger endocytosis, including opsonins or fragments thereof, and any ligand that can trigger receptor mediated endocytosis. Such ligands can vary depending on what receptors are expressed in the target cell or tissue, and include but are not limited to antibodies such as IgG, IgE, and IgA, including antibodies against receptors such as ALCAM, lipids, LDL, insulin, EGF, growth hormone, TSH, NGF, calcitonin, glucagon, prolactin, LH, TH, PDGF, interferons, catecholamines, transferrin, transcobalamin, yolk proteins, viral proteins, toxins, and any derivatives thereof. In some embodiments, the size of the liposomal nanoparticles may be selected to enhance endocytosis and release of the therapeutic agent from the HPLNs that have been taken up by the target cells.

Large numbers of therapeutic agents may be attached to one liposomal nanoparticle that may also bear from several to about one thousand targeting agents for in vivo adherence to targeted surfaces. The improved binding conveyed by multiple targeting entities can also be utilized therapeutically to block cell adhesion to endothelial receptors in vivo. Blocking these receptors can be useful to control pathological processes, such as inflammation and metastatic cancer. For example, multi-valent sialyl Lewis X derivatized nanoparticles can be used to block neutrophil binding, and antibodies against VCAM-1 on polymerized liposomal nanoparticles can be used to block lymphocyte binding, e.g. T-cells. The polymerized nanoparticle can also contain groups to control nonspecific adhesion and reticuloendothelial system uptake. For example, PEGylation of liposomes has been shown to prolong circulation lifetimes; see International Patent Application WO 90/04384, which is herein incorporated by reference in its entirety.

PLN Production

The nanoparticles described herein may be prepared in any suitable manner known to practitioners of the art, such as by sonication, shearing, hot or cold homogenization, emulsification, evaporation, spray drying, shaking of a lipid solution in the presence of an immiscible liquid, or any combinations thereof. The lipid solution may comprise therapeutic agents or other substances that are thus incorporated into the liposomal nanoparticles. In some embodiments, liposomal nanoparticles described herein are prepared through microfluidic flow focusing of a solution to be encapsulated into an aqueous solution of the encompassing lipids. If the nanoparticles produced form a population heterogeneous in size, the size of the nanoparticles may be further adjusted, such as by extrusion through a filter with a fixed pore size. In a preferred embodiment, PLNs are assembled by mixing suitable lipids and evaporating the solution to form a film. The film can then be resuspended, sonicated, and extruded through a filter.

Upon assembly, nanoparticles may be polymerized by UV light, e.g., for 2-35 minutes, or any other means for polymerization, depending on the crosslinking moiety on the polymerizable lipid(s). In some embodiments, nanoparticles may be polymerized by UV light for 2, 5, 10, 15, 20, 30, 45, 50 or 60 minutes. In some embodiments, nanoparticles may be polymerized by UV light for 1, 2, 5, 10, or 15 hours. The longer the UV exposure the more rigid the nanoparticle will generally be. The length of the UV exposure can vary depending on the composition and the application of the nanoparticles. The UV wavelength can be in the range of UV wavelength: 200-400 nm.

In some embodiments, the invention provides polymerized liposomal nanoparticles. In some embodiments, the nanoparticles comprise polymerizable lipids. In some embodiments, the nanoparticles comprise one or more lipids, at least one of which is polymerizable. Polymerizable lipids may be polymerized by any suitable method known in the art. For example, polymerizable lipids may be polymerized by addition of a catalyst to drive crosslinking, addition of a necessary linker molecule, or through photo-crosslinking, or with UV light. In some embodiments, polymerizable lipids may be polymerized with UV light.

In an exemplary embodiment, nanoparticles in aqueous solution can be distributed in wells of a 96 well plate, and dispersed with a pipette prior to UV treatment. The plate can then be placed 6 inches directly under a germicidal 30W T8 UV lamp (General Electric, Fairfield, Conn.) and be subjected to 2-5 minutes, 30 minutes or even hours of UV light.

In some embodiments, the nanoparticles described herein are produced by microfluidic flow focusing. Microfluidic flow focusing is a method for generating emulsions by flowing immiscible fluids through a small aperture, causing a pinching off of particles at regular intervals due to physical constraints. This method has been used successfully to generate microemulsions (Anna et al. 2003, Appl. Phys. Lett. 82, 364-366; Gafian-Calvo et al. 2001, Phys. Rev. Lett. 87, 274501; Garstecki et al. 2005, Phys. Rev. Lett. 94, 164501; Cubaud et al. 2005, Phys Rev. E 72, 037302). It has been shown that viscosity controls size and distribution of particles (De Menech et al. 2008, J. Fluid Mech. 595, 141-161). Thus by varying the viscosity of the lipid solution, nanoparticle size and size distribution can be varied. For water in oil emulsions, flow rate and ratio of flows have been shown to control the size of particles (Anna et al. 2003, Appl. Phys. Lett 82, 364-366). This method generates nanoparticles with size distributions subject to control through various parameters.

In some embodiments, PLNs can be assembled with components containing maleimide groups. After assembling these components into liposomes the therapeutic agent can be actively loaded and the targeting moiety attached to the maleimide group. In some embodiments, a targeting agent comprising a cysteine residue can be incubated with the loaded PLN, resulting in a covalently-linked targeting agent to the outer PLN surface.

In some embodiments, therapeutic-loaded, targeted PLNs can be prepared using pegylated liposome or polymerized PLN without using maleimide groups. This method is preferable because maleimide moieties degrade rapidly in aqueous buffers, making PLNs comprising maleimide moieties more difficult to store. Non-maleimide containing hybrid liposomes or PLNs can be similarly loaded with a therapeutic agent and optionally stored for long periods prior to adding a targeting agent.

In some embodiments, liposomal nanoparticles can be labeled with a targeting agent by exposing the nanoparticle to a labeled micelle. For example, an antibody or diabody is added as a targeting moiety to micelles as described in Iden et al., (“In vitro and in vivo comparison of immunoliposomes made by conventional coupling techniques with those made by a new post-insertion approach.” Biochimica et Biophysica Acta 1513 (2001) 207-216.) Upon preparation of the desired reduced antibody or diabody, the protein can be incubated with a fresh micelle mixture preparation containing a percentage of maleimide-terminated PEG lipids, preferably comprising lipids of the nanoparticles. The resulting antibody-conjugated micelle can then be incubated with the drug-loaded liposomal nanoparticle to allow lipid transfer to occur, which incorporates the targeting protein into the nanoparticle membrane. This method facilitates preparation and characterization of large, storable PLN batches that will not degrade with time. The micelle-forming lipids can be stored as dried powders until the targeting agent is prepared. The micelle mix can then be hydrated and incubated with the targeting agent to produce the conjugate ready for PLN insertion.

General Methods

In one aspect, the present invention relates to the fabrication and use of liposomal nanoparticles, including polymerized liposomal nanoparticles. One embodiment of the present invention involves the use of liposomal nanoparticles for the classification, diagnosis, prognosis of a condition, determination of a condition stage, determination of response to treatment, monitoring and predicting outcome of a condition. Another embodiment of the invention involves the use of the nanoparticles described herein in monitoring and predicting outcome of a condition. Another embodiment of the invention involves the use of the nanoparticles described herein in drug screening, to determine which drugs may be useful in particular diseases. Another embodiment of the invention involves the use of the nanoparticles described herein for the treatment of a condition.

The term “animal” or “animal subject” or “individual” as used herein includes humans as well as other mammals. In some embodiments, the methods involve the administration of one or more nanoparticles for the treatment of one or more conditions. Combinations of agents can be used to treat one condition or multiple conditions or to modulate the side-effects of one or more agents in the combination.

The term “treating” and its grammatical equivalents as used herein includes achieving a therapeutic benefit and/or a prophylactic benefit. By therapeutic benefit is meant eradication or amelioration of the underlying condition being treated. Also, a therapeutic benefit is achieved with the eradication or amelioration of one or more of the physiological symptoms associated with the underlying condition such that an improvement is observed in the patient, notwithstanding that the patient may still be afflicted with the underlying condition. For prophylactic benefit, the compositions may be administered to a patient at risk of developing a particular disease, or to a patient reporting one or more of the physiological symptoms of a disease, even though a diagnosis of this disease may not have been made.

As used herein the term “diagnose” or “diagnosis” of a condition includes predicting or diagnosing the condition, determining predisposition to the condition, monitoring treatment of the condition, diagnosing a therapeutic response of the disease, and prognosis of the condition, condition progression, and response to particular treatment of the condition.

In some embodiments, the invention provides methods for producing monodisperse size distribution population of liposomal nanoparticles. In some embodiments, polymerized nanoparticles are produced using traditional means such as sonication, which result in a polydisperse size distribution.

In some embodiments, the invention provides for the fabrication and use of polymerized liposomal nanoparticles (PLNs). PLNs of this invention may be used for a variety of diagnostic and therapeutic purposes, both in vivo and in vitro. In some embodiments, by varying the amount of polymerized lipid in a nanoparticle surface, the mechanical strength of the lipid surface can be increased. The PLNs may be untargeted or optionally contain targeting agents that specifically recognize target site(s), allowing for selectively enhancing imaging or therapeutic delivery of one or more therapeutic agents.

In some embodiments, the liposomal nanoparticles may be used to enhance imaging for diagnostic purposes. The nanoparticles of this invention may also contain substances as described supra to enhance imaging, e.g. for diagnostics or to visualize treatment during drug delivery.

The use of polymerizable lipids to produce liposomal nanoparticles and their use, e.g., diagnostic and therapeutic applications, provides several advantages. The use of polymerizable lipids in the making of the nanoparticles for use with clinical diagnosis or treatment offer control over properties of a contrast agent or drug/gene delivery vehicle, allowing one to modulate and optimize properties for a given application. Examples of some liposomal nanoparticles properties are mechanical elasticity, reduced nanoparticle aggregation and non-reactiveness with respect to the immune system uptake, increasing the amount of circulation time, and attaching targeting ligand.

In some embodiments, the invention provides methods that can be used to produce polymerized liposomal nanoparticles of varying size and distribution. In some embodiments, the invention provides the use of polymerized lipids in diagnostics and therapeutics purposes. In some embodiments, the invention provides for varying lipid formulations, adding PEG to the lipid head groups, or modifications of the polymerized lipid group to obtain different properties. In some embodiments, the nanoparticles can be conjugated with antibodies or peptides for targeting applications through various chemistries, or used for non-specific purposes without a targeting moiety. In some embodiments, various methods can be used for the loading of the payload, such as using a lipophilic drug that localizes in the lipid shell or covalently linking a drug to the shell for delivery applications in drug and gene therapies.

In some embodiments, the invention provides polymerized liposomal nanoparticles with increased stability. In some embodiments, the increased stability is achieved by increasing the mole fraction of polymerized lipids. In some embodiments, the nanoparticles of the invention remain intact after 2, 3, 4, 5, 7, 8, 9 10, 20, 30, 40, 50, 60, 70, 90 minutes. In some embodiments, the nanoparticles of the invention remain intact after 2, 3, 4, 5, 7, 8, 9 10, 15, 20, 30, 40, 50, 60, 70, 90 hours. In some embodiments, the nanoparticles of the invention remain intact after two days.

In some embodiments, the nanoparticles of the invention have increased circulation time. In some embodiments, the nanoparticles of the invention remain intact in circulation after 2, 3, 4, 5, 7, 8, 9 10, 20, 30, 40, 50, 60, 70, 90 minutes. In some embodiments, the nanoparticles of the invention remain intact in circulation after 2, 3, 4, 5, 7, 8, 9 10, 15, 20, 30, 40, 50, 60, 70, 90 hours. In some embodiments, the nanoparticles of the invention remain intact in circulation after two days.

In some embodiments, the nanoparticles of the invention have increased half life. In some embodiments, the nanoparticles of the invention have a half life of 2, 3, 4, 5, 7, 8, 9 10, 20, 30, 40, 50, 60, 70, 90 minutes. In some embodiments, the nanoparticles of the invention have a half life of 2, 3, 4, 5, 7, 8, 9 10, 15, 20, 30, 40, 50, 60, 70, 90 hours. In some embodiments, the nanoparticles of the invention have a half life of two days. In some embodiments, the nanoparticles of the invention have a half life of two hours. In some embodiments, 90% of the nanoparticles remain intact after 1 hr but are completely cleared from the system after 3 to 4 hr after administration. In some embodiments, the nanoparticles are completely cleared from the system not before 1 hr but are cleared after 3 to 4 hr after administration.

In some embodiments, the invention provides nanoparticles comprising a therapeutic agent. In some embodiments, the ratio by weight of therapeutic agent to lipid can be about 0.0001:1 to about 10:1, or about 0.001:1 to about 5:1, or about 0.01:1 to about 5:1, or about 0.1:1 to about 2:1, or about 0.2:1 to about 2:1, or about 0.5:1 to about 2:1, or about 0.1:1 to about 1:1. In some embodiments, the ratio by weight of therapeutic agent to lipid is 1:2. In some embodiments, the ratio by weight of therapeutic agent to lipid is 1:1. In some embodiments, the therapeutic agent is in an oil:drug phase in the lipid layer because both the oil and the therapeutic agent are hydrophobic. In some embodiments the ratio of drug to oil is 1:2. In some embodiments the ratio of drug to oil is 1:1.

In some embodiments, the invention provides nanoparticles comprising a therapeutic agent. In some embodiments, the therapeutic agent to lipid molar ratio is about 0.13 to about 0.18. In some embodiments, the therapeutic agent to lipid molar ratio is 0.15. In some embodiments, the therapeutic agent to lipid molar ratio is 0.20. In some embodiments, the therapeutic agent to lipid molar ratio is 0.25. In some embodiments, the therapeutic agent to lipid molar ratio is 0.30. In some embodiments, the therapeutic agent to lipid molar ratio is 0.45. In some embodiments, the therapeutic agent to lipid molar ratio is 0.50.

In some embodiments, the nanoparticles of the invention retain the therapeutic agent under physiological conditions. In some embodiments, the nanoparticles of the invention retain 50%, 55%, 60%, 70%, 80%, 90%, 95%, 99% of the therapeutic agent. In some embodiments, the nanoparticles of the invention retain at least 70% of the therapeutic agent. In some embodiments, the nanoparticles of the invention retain 80% of the therapeutic agent. In some embodiments, the nanoparticles of the invention retain 90% of the therapeutic agent. In some embodiments, the nanoparticles of the invention retain 100% of the therapeutic agent.

In some embodiments, without intending to be limited to any theory, polymerization prevents the therapeutic agent leakage for days under physiological conditions. The partially or completely polymerized nanoparticles of the invention are stable against leakage yet capable of instantaneous release for remote controlled drug delivery: Polymerization can increase stability in solution, offering greater mechanical stability to help counter nanoparticle destruction. The dissolution rate is tunable by controlling the amount of polymer in the shell.

In some embodiments, nanoparticle properties are optimized to maximize efficiency for a given application, increasing or decreasing stiffness to maximize binding at a target site or modulating stability to optimize gene delivery.

In some embodiments, the present invention provides for a nanoparticle comprising a polymerized lipid shell and a liquid, wherein the liquid is encased with the shell. In some embodiments, the nanoparticles of the invention comprise one or more polymerizable lipid. Examples of polymerizable lipids include but are not limited to, diyne PC and diynePE, for example 1,2-bis(10,12-tricosadiynoyl)-sn-glycero-3-phosphocoline. In one embodiment, the polymerized lipid shell of the nanoparticle comprises at least one polymerizable lipid and at least one non-polymerizable lipid and has a percentage of about 5-50% polymerizable lipid. In some embodiments, the percentage of polymerizable lipid is 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 of the total lipid mixture of making the nanoparticles. In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of polymerizable lipids. In some embodiments, the nanoparticles of the invention comprise at least 25% of polymerizable lipids. In some embodiments, the nanoparticles of the invention comprise at least 50% of polymerizable lipids. In some embodiments, the nanoparticles of the invention comprise about 15% to about 20% of polymerizable lipids. In one embodiment, the at least one polymerizable lipid is a diacetylenic lipid

In some embodiments, the nanoparticles of the invention comprise one or more negatively charged phospholipids. Examples of negatively charged phospholipids include, but are not limited to, dipalmitoyl and distearoyl phosphatidic acid (DPPA, DSPA), dipalmitoyl and distearoyl phosphatidylserine (DPPS, DSPS), phosphatidyl glycerols such as dipalmitoyl and distearoyl phosphatidylglycerol (DPPG, DSPG).

In one embodiment, the at least one non-polymerizable lipid is selected group the group of L-α-phosphatidylcholine, PE-PEG2000 (1,2-distearoyl-sn-glycero-3-phosphoethanolamine-N-[methoxy(polyethylene glycol)-2000 or PE-PEG2000-biotin. In one embodiment, the polymerized lipid shell comprises a percentage of PEGylated lipid between about 1-20%. In some embodiments, the percentage of PEGylated lipid in the nanoparticle is 1, 2, 3, 4, 5, 6, 7, 8, 9, 10, 11, 12, 13, 14, 15, 16, 17, 18, 19, or 20. In one embodiment, the lipid is non-polymerizable and PEGylated. In one embodiment, the lipid is polymerizable and PEGylated.

In some embodiments, the nanoparticles of the invention comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged lipids. In some embodiments, the nanoparticles of the invention comprise at least 2% of negatively charged lipids. In some embodiments, the nanoparticles of the invention comprise at least 5% of negatively charged lipids. In some embodiments, the nanoparticles of the invention comprise at least 10% of negatively charged lipids. In some embodiments, the nanoparticles of the invention comprise at least 25% of negatively charged lipids. In some embodiments, the nanoparticles of the invention comprise at least 30% of negatively charged lipids. In some embodiments, the nanoparticles of the invention comprise about 1% to about 15% of negatively charged lipids. In some embodiments, the nanoparticles of the invention comprise about 5% of negatively charged lipids. In some embodiments, the nanoparticles of the invention comprise about 6% of negatively charged lipids.

In some embodiments, the nanoparticles of the invention comprise about 2% of negatively charged lipids and about 15% to about 20% of a polymerizable lipid. In some embodiments, the nanoparticles of the invention comprise about 5% of negatively charged lipids and about 15% to about 20% of a polymerizable lipid. In some embodiments, the nanoparticles of the invention comprise about 10% of negatively charged lipids and about 15% to about 20% of a polymerizable lipid. In some embodiments, the nanoparticles of the invention comprise about 25% of negatively charged lipids and about 15% to about 20% of a polymerizable lipid. In some embodiments, the nanoparticles of the invention comprise about 30% of negatively charged lipids and about 15% to about 20% of a polymerizable lipid. In some embodiments, the nanoparticles of the invention comprise the same percentage of negatively charged lipids and polymerizable lipids. In some embodiments, the negatively charged lipid and the polymerizable lipid is the same. In some embodiments, the nanoparticles of the invention comprise at least two negatively charged lipids, but only one of the two negatively charged lipids is polymerizable.

In some embodiments, the nanoparticles of the invention comprise about 15% to about 20% of polymerizable lipid, about 1-15% of negatively charged lipid, about 20-45% of neutrally charged lipid and about 30 to 60% of zwitterionically charged lipid. In some embodiments, the polymerizable lipid is a C25 tail lipid. In some embodiments, the negatively charged lipid is a C18 tail lipid. In some embodiments, the zwitterionically charged lipid is a C18 tail lipid. In some embodiments, the polymerizable lipid is a diacetylenic lipid.

In some embodiments, the nanoparticles of the invention comprise about 15 to about 20% of 10,12-pentacosadiynoic acid derivatives and about 30% to about 40% of saturated phospholipids. In some embodiments, the nanoparticles of the invention comprise about 15% of C25 tail lipid and about 50 to about 55% of C18 tail lipid.

In some embodiments, the nanoparticles of the invention comprise a ratio of 3.5:1 of at least two lipids that differ in tail size by at least 7 carbons. In some embodiments, one lipid(s) is a C25 tail lipid and the other lipid(s) is a C18 tail lipid.

In some embodiments, the nanoparticles of the invention comprise a targeting agent and a therapeutic agent, where the polymerized lipid nanoparticle has a potency of at least 2 fold higher than conventional liposome pegylated preparation. In some embodiments, the nanoparticles of the invention comprise a targeting agent and a therapeutic agent, wherein therapeutic agent to lipid molar ratio is 0.15.

In one embodiment, the nanoparticle is conjugated with a targeting agent (e.g. a cell receptor ligand) and the conjugation is by any way of the tethering the targeting agent to the lipid shell. Methods of tethering targeting agents to liposomal nanoparticles are well known in the art, e.g. using carbodiimide, maleimide, or biotin [symbol] streptavidin coupling (Klibanov 2005, Bioconjug. Chem. 16, 9-17). Biotin-streptavidin is the most popular coupling strategy because biotin's affinity for streptavidin is very strong and it is easy to label ligands with biotin. In some embodiments, targeting agents include monoclonal antibodies and other ligands that bind to receptors (e.g. VCAM-1, ICAM-1, E-selection) expressed by the cell type of interest, e.g. inflammatory cells, vasculature cells, or tumor cells.

In some embodiments, the targeting agent is selected from a group consisting of antibodies, ligands, proteins, peptides, carbohydrates, vitamins, nucleic acids and combinations thereof. In some embodiments, the targeting agent is specific to a cell surface Molecule. In some embodiments, the targeting agent enhances endocytosis and/or cell membrane fusion.

In one embodiment, the nanoparticles encapsulate a therapeutic agent (e.g., drug or chemical) or any entity within the shell. In some embodiments, the therapeutic agent or entity within the shell is delivered to a target location by way of the nanoparticle.

In some embodiments, the polymerized lipid nanoparticle is internalized into the endosome compartment of a target cell after about 1, 5, 10, 20, 25, 30, 45, 50, 55, 60 minutes of administration to a subject. In some embodiments, the polymerized lipid nanoparticle is internalized into the endosome compartment of a target cell after about 1, 1.5, 2, 2.5, 3, 3.5 or 4 hours of administration to a subject. In some embodiments, the polymerized lipid nanoparticle is internalized into the endosome compartment of a target cell after about 30 minutes of administration to a subject. In some embodiments, the polymerized lipid nanoparticle is internalized into the endosome compartment of a target cell after about 60 minutes of administration to a subject. In some embodiments, the polymerized lipid nanoparticle is internalized into the endosome compartment of a target cell after about 2 hours of administration to a subject. In some embodiments, the polymerized lipid nanoparticle is internalized into the endosome compartment of a target cell after about 4 hours of administration to a subject.

In some embodiments, the therapeutic agent in the polymerized lipid nanoparticle is released into a target cell after about 1, 5, 10, 20, 25, 30, 45, 50, 55, 60 minutes of internalization into the target cell. In some embodiments, the therapeutic agent in the polymerized lipid nanoparticle is released into a target cell after about 1, 1.5, 2, 2.5, 3, 3.5 or 4 hours of internalization into the target cell.

In one embodiment, the nanoparticle is UV treated for about 2-35 minutes after fabrication to polymerize the lipid shell. It is understood that one can UV treat the formed nanoparticles for a time period of anywhere from 2.0 min to several hours in order to achieve various/desired level of polymerization in the shell. In some embodiments, the nanoparticle is UV treated for about 2, 5, 10, 15, 20, 30, 45, 50 or 60 minutes. In some embodiments, the nanoparticle is UV treated for about 1, 2, 5, 10, or 15 hours. The UV wavelength can be in the range of UV wavelength: 200-400 nm. The shell material affects nanoparticle mechanical elasticity. The level of polymerization of the shell affects the mechanical elasticity. By varying the UV treatment timing, the amount of polymerization of the shell can be adjusted, e. g. 2 or 3 min for lower polymerization, 4-30 min for higher polymerization. In one embodiment, the nanoparticle is UV treated for about 2 minutes. In another embodiment, the nanoparticle is UV treated for about 3 minutes. In another embodiment, the nanoparticle is UV treated for about 4 minutes. In another embodiment, the nanoparticle is UV treated for about 5 minutes. In other embodiments, the nanoparticle is UV treated for about 2.1, 2.2, 2.3, 2.4, 2.5, 2.6, 2.7, 2.8, 2.9, 3.1, 3.2, 3.3, 3.4, 3.5, 3.6, 3.7, 3.8, 3.9, 4.1, 4.2, 4.3, 4.4, 4.5, 4.6, 4.7, 4.8, or 4.9 minutes. In another embodiment, the nanoparticle is UV treated for about 10 minutes. In another embodiment, the nanoparticle is UV treated for about 20 minutes. In another embodiment, the nanoparticle is UV treated for about 30 minutes. In another embodiment, the nanoparticle is UV treated for about 60 minutes. In another embodiment, the nanoparticle is UV treated for about 2 hours.

In one embodiment, the nanoparticle has an absorbance at a wavelength between about 400-580 μm. In some embodiments, the nanoparticle has an absorbance at a wavelength of about 400, 410, 420, 430, 440, 450, 460, 470, 480, 490, 500, 510, 520, 530, 540, 550, 560, 570, or 580 μm. In one embodiment, the absorbance at a wavelength between about 400-580 nm is an indication of the successful polymerization of the polymerizable lipid forming the shell of the nanoparticle. This is especially so when the polymerizable lipid is a diacetylenic lipid. In another embodiment, the nanoparticle appears to be blue or purple, wherein blue indicates one form of polymerized diacetylenic lipid and purple indicates a mixture of a red and a blue form of polymerized diacetylenic lipid.

In one embodiment, the collection of liposomal nanoparticles is monodispersed, and the monodisperity is about 20% of an average size of the nanoparticles in the collection. In one embodiment, the collection of nanoparticles is monodispersed, and the monodisperity is about 15% of an average size of the nanoparticles in the collection. In one embodiment, the collection of nanoparticles is monodispersed, and the monodisperity is about 10% of an average size of the nanoparticles in the collection. In one embodiment, the collection of nanoparticles is monodispersed, and the monodisperity is about 5% of an average size of the nanoparticles in the collection. In one embodiment, the collection of nanoparticles is monodispersed, and the monodisperity is about 1% of an average size of the nanoparticles in the collection.

In some embodiments, 90% of the nanoparticles in the collection remain intact after 2, 3, 4, 5, 7, 8, 9 10, 20, 30, 40, 50, 60, 70, 90 minutes. In some embodiments, 90% of the nanoparticles in the collection remain intact after 2, 3, 4, 5, 7, 8, 9 10, 15, 20, 30, 40, 50, 60, 70, 90 hours. In some embodiments, 90% of the nanoparticles in the collection remain intact after two days. In some embodiments, 80% of the nanoparticles in the collection remain intact after 2, 3, 4, 5, 7, 8, 9 10, 20, 30, 40, 50, 60, 70, 90 minutes. In some embodiments, 80% of the nanoparticles in the collection remain intact after 2, 3, 4, 5, 7, 8, 9 10, 15, 20, 30, 40, 50, 60, 70, 90 hours. In some embodiments, 80% of the nanoparticles in the collection remain intact after two days. In some embodiments, 50% of the nanoparticles in the collection remain intact after 2, 3, 4, 5, 7, 8, 9 10, 20, 30, 40, 50, 60, 70, 90 minutes. In some embodiments, 50% of the nanoparticles in the collection remain intact after 2, 3, 4, 5, 7, 8, 9 10, 15, 20, 30, 40, 50, 60, 70, 90 hours. In some embodiments, 50% of the nanoparticles in the collection remain intact after two days. In some embodiments, 90% of the nanoparticles in the collection remain intact after 90 minutes. In some embodiments, 50% of the nanoparticles in the collection remain intact after 15 hours. In some embodiments, 50% of the nanoparticles in the collection remain intact after two days.

In some embodiments, the collection of nanoparticles of the invention has increased circulation time. In some embodiments, the collection of nanoparticles of the invention remains intact in circulation after 2, 3, 4, 5, 7, 8, 9 10, 20, 30, 40, 50, 60, 70, 90 minutes. In some embodiments, the collection of nanoparticles of the invention remains intact in circulation after 2, 3, 4, 5, 7, 8, 9 10, 15, 20, 30, 40, 50, 60, 70, 90 hours. In some embodiments, the collection of nanoparticles of the invention remains intact in circulation after two days. In some embodiments, 90% of the collection of nanoparticles of the invention remains intact in circulation after about 3 to 4 hours.

In some embodiments, the collection of nanoparticles of the invention has increased half life. In some embodiments, the collection of nanoparticles of the invention has a half life of 2, 3, 4, 5, 7, 8, 9 10, 20, 30, 40, 50, 60, 70, 90 minutes. In some embodiments, the collection of nanoparticles of the invention has a half life of 2, 3, 4, 5, 7, 8, 9 10, 15, 20, 30, 40, 50, 60, 70, 90 hours. In some embodiments, the collection of nanoparticles of the invention has a half life of two days. In some embodiments, of the collection of nanoparticles of the invention remains intact in circulation after about 3 to 4 hours, but then are cleared from the system.

In some embodiments, the average size of the nanoparticles in the collection is between about 30 nm-5 μm. In some embodiments, the average size of the nanoparticles in the collection is between about 50 nm-5 μm. In some embodiments, the average size of the nanoparticles in the collection is between about 30 nm-1.5 μm. In some embodiments, the average size of the nanoparticles in the collection is between about 50 nm-120 nm. In some embodiments, the average size of the nanoparticles in the collection is about 90 nm. In some embodiments, the average size of the nanoparticles in the collection is about 100 nm. In another embodiment, the average size of the nanoparticles in the collection is about 110 nm. In other embodiments, the average size of the nanoparticles in the collection is about 90, 91, 92, 93, 94, 95, 100, 101, 102, 103, 105, 106, or 110 nm.

In some embodiments, the nanoparticles of the collection comprise about 15% of polymerizable lipids. In some embodiments, the nanoparticles of the collection comprise about 20% of polymerizable lipids. In one embodiment, the at least one polymerizable lipid is a diacetylenic lipid

In some embodiments, the nanoparticles of the collection comprise one or more negatively charged lipids. In one embodiment, the nanoparticles of the collection comprise a percentage of PEGylated lipids between about 1-20%.

In some embodiments, the nanoparticles of the collection comprise at least 0.1%, 0.5%, 1%, 5%, 10%, 15%, 20%, 25%, 30%, 35%, 40%, 45%, 50%, 55%, 60%, 70%, 80%, 90% or 100% of negatively charged lipids. In some embodiments, the nanoparticles of the collection comprise at least 2% of negatively charged lipids. In some embodiments, the nanoparticles of the collection comprise at least 5% of negatively charged lipids. In some embodiments, the nanoparticles of the collection comprise at least 10% of negatively charged lipids. In some embodiments, the nanoparticles of the collection comprise at least 25% of negatively charged lipids. In some embodiments, the nanoparticles of the collection comprise at least 30% of negatively charged lipids.

In some embodiments, the nanoparticles of the collection comprise nanoparticles as described herein.

Drug Delivery

In some embodiments, one or more therapeutic agents may be attached to the surface of the nanoparticle, incorporated in the lipid layer, or trapped within the lipid shell. Nanoparticles may further include a targeting agent or agents that recruit the nanoparticle to a target site.

Therapeutic agents may optionally be freed from the nanoparticles without destroying the nanoparticle. The nanoparticles can be vibrated or disrupted to allow agents enclosed within the nanoparticle to pass through the lipid membrane. Agents attached to the lipids may be cleaved from the nanoparticle surface, such as by enzymatic hydrolysis.

In some embodiments, liposomal nanoparticles can be endocytosed or phagocytosed by a target cell. Nanoparticles can comprise markers, such as membrane proteins, that enhance endocytosis. In a preferred embodiment, the marker is ALCAM. Upon endocytosis, nanoparticles are present in membrane vesicles, such as endosomes or lysosomes of the target cell. In some embodiments, nanoparticles can then fuse with the endosomal membrane to release their internal contents into the cytoplasm. In some embodiments, nanoparticles can be disrupted inside the endosome to release their contents into the endosome, where the drug or other therapeutic agent can then diffuse or be transported from the endosome to other cellular compartments or the cytoplasm. In some embodiments, the drugs or other therapeutic agents can be released from the nanoparticle upon a decrease in pH, as can occur as endosomes progress through the endocytic pathway, e.g. to become late endosomes or lysosomes. In some embodiments, the drugs or other therapeutic agents can be released from the nanoparticle by enzymatic activity, such as by a lysosomal hydrolase. Such release can occur, for example, by breaking the bond between the drug or therapeutic agent with the nanoparticle, or by disrupting the entire nanoparticle. In some embodiments, the drug or therapeutic agent can diffuse from the nanoparticle into the rest of the membrane vesicle, and optionally from the vesicle into other regions of the cell.

In some embodiments, liposomal nanoparticles can fuse with the cellular membrane. In some embodiments, membrane fusion can directly release the internal contents of the nanoparticles into the cellular cytoplasm. In some embodiments, the drug or therapeutic agent can be attached, covalently or non-covalently, to a component of the nanoparticle membrane, and can be incorporated into the cellular membrane upon membrane fusion. The drug or therapeutic agent can then optionally be internalized into the cell, for example by receptor endocytosis or active transport.

In some embodiments, the lipid composition of the nanoparticle can be selected to improve drug loading or delivery. In some embodiments, PCDA lipid content can be increased to increase nanoparticle potency. In some embodiments, the polymerizable lipid concentration can be less than about 30%, less than about 25%, less than about 20%, less than about 15%, less than about 10%, or less than about 5% to improve drug loading efficiency.

Compositions

The present invention is also directed toward therapeutic/diagnostic compositions comprising the therapeutic/diagnostic agents of the present invention. The sizes of the nanoparticles may be different for different applications. For general vascular imaging and therapy, sizes may range from about 30 nm to about 10 μm in diameter, preferably between about 2 μm and about 5 μm in diameter. In some embodiments, sizes may range from about 2 μm to about 4 μm. In some embodiments, for applications in tumors or in organs such as the liver, smaller nanoparticles (less than 2 μm in diameter) are preferred. Larger nanoparticles may be used for imaging or delivery intrarectally or intranasally, up to about 100 μm in diameter.

In some embodiments, the therapeutic delivery systems of the invention are administered in the form of an aqueous suspension such as in water or a saline solution (e.g., phosphate buffered saline). Preferably, the water is sterile. Also, preferably the saline solution is an isotonic saline solution, although, if desired, the saline solution may be hypotonic (e.g., about 0.3 to about 0.5% NaCl). The solution may also be buffered, if desired, to provide a pH range of about pH 5 to about pH 7.4. In addition, dextrose may be preferably included in the media. Further solutions that may be used for administration of PSMs include, but are not limited to almond oil, corn oil, cottonseed oil, ethyl oleate, isopropyl myristate, isopropyl palmitate, mineral oil, myristyl alcohol, octyldodecanol, olive oil, peanut oil, persic oil, sesame oil, soybean oil, and squalene.

Compositions of the present invention can also include other components such as a pharmaceutically acceptable excipient, an adjuvant, and/or a carrier. For example, compositions of the present invention can be formulated in an excipient that the animal to be treated can tolerate. Examples of such excipients include water, saline, Ringer's solution, dextrose solution, mannitol, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer, Tris buffer, histidine, citrate, and glycine, or mixtures thereof, while examples of preservatives include thimerosal, m- or o-cresol, formalin and benzyl alcohol. Standard formulations can either be liquid injectables or solids which can be taken up in a suitable liquid as a suspension or solution for injection. Thus, in a non-liquid formulation, the excipient can comprise dextrose, human serum albumin, preservatives, etc., to which sterile water or saline can be added prior to administration.

In one embodiment of the present invention, the composition can also include an immunopotentiator, such as an adjuvant or a carrier. Adjuvants are typically substances that generally enhance the immune response of an animal to a specific antigen. Suitable adjuvants include, but are not limited to, Freund's adjuvant; other bacterial cell wall components; aluminum-based salts; calcium-based salts; silica; polynucleotides; toxoids; serum proteins; viral coat proteins; other bacterial-derived preparations; gamma interferon; block copolymer adjuvants, such as Hunter's Titermax adjuvant (Vaxcel™, Inc. Norcross, Ga.); Ribi adjuvants (available from Ribi ImmunoChem Research, Inc., Hamilton, Mont.); and saponins and their derivatives, such as Quil A (available from Superfos Biosector A/S, Denmark). Carriers are typically compounds that increase the half-life of a therapeutic composition in the treated animal. Suitable carriers include, but are not limited to, polymeric controlled release formulations, biodegradable implants, liposomes, bacteria, viruses, oils, esters, and glycols.

One embodiment of the present invention is a controlled release formulation that is capable of slowly releasing a composition of the present invention into an animal. As used herein, a controlled release formulation comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Other controlled release formulations of the present invention include liquids that, upon administration to an animal, form a solid or a gel in situ. Preferred controlled release formulations are biodegradable (i.e., bioerodible).

Generally, the therapeutic/diagnostic agents used in the invention are administered to an animal in an effective amount. Generally, an effective amount is an amount effective to (1) reduce the symptoms of the condition sought to be treated, (2) induce a pharmacological change relevant to treating the condition sought to be treated or (3) detect the nanoparticles in vivo or in vitro. For cancer, for example, an effective amount includes an amount effective to: reduce the size of a tumor, slow the growth of a tumor; prevent or inhibit metastases; or increase the life expectancy of the affected animal.

Effective amounts of the therapeutic/diagnostic agents can be any amount or doses sufficient to bring about the desired effect and depend, in part, on the condition, type and location of the cancer, the size and condition of the patient, as well as other factors readily known to those skilled in the art. The dosages can be given as a single dose, or as several doses, for example, divided over the course of several weeks.

The present invention is also directed toward methods of treatment utilizing the therapeutic compositions of the present invention. The method comprises administering the therapeutic agent to a subject in need of such administration.

The therapeutic agents of the instant invention can be administered by any suitable means as described herein, including, for example, parenteral, topical, oral or local administration, such as intradermally, by injection, or by aerosol. In the preferred embodiment of the invention, the agent is administered by injection. Such injection can be locally administered to any affected area. A therapeutic composition can be administered in a variety of unit dosage forms depending upon the method of administration. For example, unit dosage forms suitable for oral administration of an animal include powder, tablets, pills and capsules. Preferred delivery methods for a therapeutic composition of the present invention include intravenous administration and local administration by, for example, injection or topical administration. For particular modes of delivery, a therapeutic composition of the present invention can be formulated in an excipient of the present invention. A therapeutic reagent of the present invention can be administered to any animal, preferably to mammals, and more preferably to humans.

The particular mode of administration will depend on the condition to be treated. It is contemplated that administration of the agents of the present invention may be via any bodily fluid, or any target or any tissue accessible through a body fluid.

Preferred routes of administration of the cell-surface targeted therapeutic agents of the present invention are by intravenous, interperitoneal, or subcutaneous injection including administration to veins or the lymphatic system. A targeted agent can be designed to focus on markers present in any fluids, body tissues; and body cavities, e.g. synovial fluid, ocular fluid, or spinal fluid. Thus, for example, an agent can be administered to spinal fluid, where an antibody targets a site of pathology accessible from the spinal fluid. Intrathecal delivery, that is, administration into the cerebrospinal fluid bathing the spinal cord and brain, may be appropriate for example, in the case of a target residing in the choroid plexus endothelium of the cerebral spinal fluid (CSF)-blood barrier.

As an example of one treatment route of administration through a bodily fluid is one in which the condition to be treated is rheumatoid arthritis. In this embodiment of the invention, the invention provides therapeutic agents to treat inflamed synovia of people afflicted with rheumatoid arthritis. This type of therapeutic agent is a radiation synovectomy agent. The route of administration through the synovia may also be useful in the treatment of osteoarthritis. Delivery of agents by injection of targeted carriers to synovial fluid to reduce inflammation, inhibit degradative enzymes, and decrease pain is envisioned in some embodiments of the invention.

Another route of administration is through ocular fluid. When the vasculature of the eye is targeted, it should be appreciated that targets may be present on either side of the vasculature. Delivery of the agents of the present invention to the tissues of the eye can be in many forms, including intravenous, ophthalmic, and topical. For ophthalmic topical administration, the agents of the present invention can be prepared in the form of aqueous eye drops such as aqueous suspended eye drops, viscous eye drops, gel, aqueous solution, emulsion, ointment, and the like. Additives suitable for the preparation of such formulations are known to those skilled in the art. In the case of a sustained-release delivery system for the eye, the sustained-release delivery system may be placed under the eyelid or injected into the conjunctiva, sclera, retina, optic nerve sheath, or in an intraocular or intraorbitol location. Intravitreal delivery of agents to the eye is also contemplated. Such intravitreal delivery methods are known to those of skill in the art. The delivery may include delivery via a device, such as that described in U.S. Pat. No. 6,251,090 to Avery.

In a further embodiment, the therapeutic agents of the present invention are useful for gene therapy. As used herein, the phrase “gene therapy” refers to the transfer of genetic material (e.g., DNA or RNA) of interest into a host to treat or prevent a genetic or acquired condition. The genetic material of interest encodes a product (e.g., a protein polypeptide, peptide or functional RNA) whose production in vivo is desired. For example, the genetic material of interest can encode a hormone, receptor, enzyme or polypeptide of therapeutic value. In a specific embodiment, the subject invention utilizes a class of lipid molecules for use in non-viral gene therapy which can complex with nucleic acids as described in Hughes, et al., U.S. Pat. No. 6,169,078, incorporated by reference herein in its entirety, in which a disulfide linker is provided between a polar head group and a lipophilic tail group of a lipid.

These therapeutic compounds of the present invention effectively complex with DNA and facilitate the transfer of DNA through a cell membrane into the intracellular space of a cell to be transformed with heterologous DNA. Furthermore, these lipid molecules facilitate the release of heterologous DNA in the cell cytoplasm thereby increasing gene transfection during gene therapy in a human or animal.

Liposomal nanoparticles of this invention may be stored dry or suspended in a variety of liquid solutions, including distilled water or in aqueous solutions. Aqueous solutions may be buffered to suitable pH ranges (about 5 to about 7.4) by HEPES, Tris, phosphate, acetate, citrate, phosphate, bicarbonate, or other buffers, and may contain isotonic (about 0.9% NaCl) or hypotonic (about 0.3 to about 0.5% NaCl) salt concentrations.

The solutions may also include emulsifying and/or solubilizing agents. Such agents include, but are not limited to, acacia, cholesterol, diethanolamine, glyceryl monostearate, lanolin alcohols, lecithin, mono- and di-glycerides, mono-ethanolamine, oleic acid, oleyl alcohol, poloxamer, polyoxyethylene 50 stearate, polyoxyl 35 castor oil, polyoxyl 10 oleyl ether, polyoxyl 20 cetostearyl ether, polyoxyl 40 stearate, polysorbate 20, polysorbate 40, polysorbate 60, polysorbate 80, propyleneglycol diacetate, propylene glycol monostearate, sodium lauryl sulfate, sodium stearate, sorbitan mono-laurate, sorbitan mono-oleate, sorbitan mono-palmitate, sorbitan monostearate, stearic acid, trolamine, and emulsifying wax. Suspending and/or viscosity-increasing agents that may be used with lipid or nanoparticle solutions include but are not limited to, acacia, agar, alginic acid, aluminum monostearate, bentonite, magma, carbomer 934P, carboxymethylcellulose, calcium and sodium and sodium 12, carrageenan, cellulose, dextrin, gelatin, guar gum, hydroxyethyl cellulose, hydroxypropyl methylcellulose, magnesium aluminum silicate, methylcellulose, pectin, polyethylene oxide, polyvinyl alcohol, povidone, propylene glycol alginate, silicon dioxide, sodium alginate, tragacanth, and xanthum gum.

Bacteriostatic agents may also be included with the nanoparticles to prevent bacterial degradation on storage. Suitable bacteriostatic agents include but are not limited to benzalkonium chloride, benzethonium chloride, benzoic acid, benzyl alcohol, butylparaben, cetylpyridinium chloride, chlorobutanol, chlorocresol, methylparaben, phenol, potassium benzoate, potassium sorbate, sodium benzoate and sorbic acid.

Administration

The methods involve the administration of one or more nanoparticles, e.g., for the diagnosis and/or treatment of a condition. In some embodiments, other agents are also administered, e.g., other therapeutic agent. When two or more agents are co-administered, they may be co-administered in any suitable manner, e.g., as separate compositions, in the same composition, by the same or by different routes of administration.

The nanoparticles of this invention may be administered in a variety of methods, such as intravascularly, intralymphatically, parenterally, subcutaneously, intramuscularly, intranasally, intrarectally, intraperitoneally, interstitially, into the airways via nebulizer, hyperbarically, orally, topically, or

intratumorly, using a variety of dosage forms. In some embodiments, the nanoparticles are injected intravenously. In some embodiments, the nanoparticles are injected intraarterially. The nanoparticles may also be utilized in vitro, such as may be useful for diagnosis using tissue biopsies.

In some embodiments, the nanoparticles are administered in a single dose, e.g., for the treatment of an acute condition. Typically, such administration will be by injection. However, other routes may be used as appropriate. In some embodiments, the nanoparticles are administered in multiple doses. Dosing may be about once, twice, three times, four times, five times, six times, or more than six times per day. Dosing may be about once a month, once every two weeks, once a week, or once every other day. In one embodiment the nanoparticles are administered about once per day to about 6 times per day. In another embodiment the administration of the nanoparticles continue for less than about 7 days. In yet another embodiment the administration continues for more than about 6, 10, 14, 28 days, two months, six months, or one year. In some cases, continuous dosing is achieved and maintained as long as necessary. In some embodiments, the nanoparticles are administered continually or in a pulsatile manner, e.g. with a minipump, patch or stent.

Administration of the nanoparticles of the invention may continue as long as necessary. In some embodiments, an agent of the invention is administered for more than 1, 2, 3, 4, 5, 6, 7, 14, 28 days or 1 year. In some embodiments, an agent of the invention is administered for less than 28, 14, 7, 6, 5, 4, 3, 2, or 1 day. In some embodiments, an agent of the invention is administered chronically on an ongoing basis, e.g., for the treatment of chronic effects.

When diagnosis and/or treatment need to be performed as a series, e.g., a series of diagnostic tests after treatment, the diagnosis and/or treatment may be performed at fixed intervals, at intervals determined by the status of the most recent diagnostic test or tests or by other characteristics of the individual, or some combination thereof. For example, diagnosis and/or treatment may be performed at intervals of approximately 1, 2, 3, or 4 weeks, at intervals of approximately 1, 2, 3, 4, 5, 6, 7, 8, 9, 10, or 11 months, at intervals of approximately 1, 2, 3, 4, 5, or more than 5 years, or some combination thereof. It will be appreciated that an interval may not be exact, according to an individual's availability for diagnosis and/or treatment and the availability of diagnostic/treatment facilities, thus approximate intervals corresponding to an intended interval scheme are encompassed by the invention. As an example, an individual who has undergone treatment for a cancer may be tested/treated relatively frequently (e.g., every month or every three months) for the first six months to a year after treatment, then, if no abnormality is found, less frequently (e.g., at times between six months and a year) thereafter. If, however, any abnormalities or other circumstances are found in any of the intervening times, intervals may be modified.

In one embodiment, a diagnostic test may be performed on an apparently healthy individual during a routine checkup and analyzed so as to provide an assessment of the individual's general health status. In another embodiment, a diagnostic test may be performed to screen for commonly occurring diseases. Such screening may encompass testing for a single disease, a family of related diseases or a general screening for multiple, unrelated diseases. Screening can be performed weekly, bi-weekly, monthly, bi-monthly, every several months, annually, or in several year intervals and may replace or complement existing screening modalities.

Progression in the circulation of the administered nanoparticle formulation toward the selected site may be monitored any suitable method known in the art, including those described herein, e.g., by ultrasonic imaging means, or by MRI or radiography if the formulation includes agents for such imaging. In some embodiments, the circulation of the administered nanoparticle formulation toward the selected site is monitored using ultrasonic imaging means. The ultrasonic irradiation may be carried out by a modified echography probe adapted to simultaneously monitor the reflected echo signal and thereby provide an image of the irradiated site. The monitoring signal can be in the range of 1 MHz to 10 MHz and preferably between 2 and 7 MHz.

The useful dosage of lipid nanoparticles to be administered and the mode of administration will vary depending upon the age, weight, and mammal to be treated, and the particular application (therapeutic/diagnostic) intended. Typically, dosage is initiated at lower levels and increased until the desired therapeutic effect or diagnostic sensitivity is achieved.

Conditions

In some embodiments, the invention provides compositions and methods for the diagnosis and/or treatment of a condition.

In some embodiments, nanoparticles of the invention can be used with MRI or other imaging techniques, e.g. to visualize tumors. Liposomal nanoparticle usage for therapeutic purposes is potentially limited only by the drugs or other therapeutic agents that can be linked to the microbubbles. As some non-limiting examples, nanoparticles linked to antiangiogenics may be used to treat tumors, nanoparticles linked to anti-atherosclerotic drugs may be used to treat plaques in the vasculature, or nanoparticles linked to local anesthetics may be used to anesthetize a specific area region of interest: Because nanoparticles, such as PLNs, can be extremely stable, they may also be administered for use as slow-release capsules to provide a constant, preferably low dosage of a therapeutic agent.

The methods, systems and compositions described herein can be used for the diagnosis and treatment of conditions, e.g., atherosclerosis. Atherosclerosis is the chronic inflammation of the arteries, which through plaque formation and rupture can result in heart attack and stroke. Studies have shown that the vast majority of adults in the United States have atherosclerotic lesions (Tuzcu et al. 2001, Circulation 103, 2705-2710). Current diagnostic techniques concentrate on the size of plaques to determine the risk to the patient. However, plaques vulnerable to rupture differ from stable plaques in molecular composition, not size (Virmani et al. 2006, J. Am. Coll. of Card. 47, C13-18). For this reason, molecular imaging of the cardiovascular system offers a means of identifying vulnerable plaques and would be a substantial improvement over current techniques. Contrast agents for enhancing MRI detection can be loaded into nanoparticles targeted to atherosclerotic plaques for targeted imaging of plaques.

In addition to applications in imaging, targeted liposomal nanoparticles could be used in the circulatory system for drug delivery applications. Following angioplasty and/or stenting for the treatment of arterial occlusion, restenosis often occurs, causing the artery to become occluded again. For example, following carotid angioplasty and stenting, the restenosis rate after one year is approximately 6 percent (Groschel et al. 2005, Stroke 36, 367-373). Drug eluting stents have decreased the risk of restenosis, but cause long-term safety concerns, because of potential thrombogenicity and inflammation. Late in-stent thrombosis may be higher in drug-eluting stents, with one report recording four times the incidence relative to bare metal stents after one year (Carlsson et al. 2007, Clin. Res. Cardiol. 96, 86-93). Alternative methods of paclitaxel delivery to sites of inflammation are a current topic of research, in order to prevent the need for the placement of additional stents, which may exacerbate the problem (Herdeg et al. 2008 Thrombosis Res. 123, 236-243; Spargias et al. 2009 J. Interv. Cardiol. 22, 291-298; Unverdorben et al. 2009 Circulation 119, 2986-2994). Nanoparticles of the invention can be used to selectively deliver drugs, e.g. paclitaxel, to potential restonosis sites. This non-invasive method could provide a safer delivery tool to prevent restenosis at damaged sites.

In some embodiments, the nanoparticles are used for the treatment of an inflammatory condition. For instance, the nanoparticles can be used to treat Encephalomyelitis. Further, in other embodiments the nanoparticles are used for the treatment of obstructive pulmonary disease. This is a disease state characterized by airflow limitation that is not fully reversible. The airflow limitation is usually both progressive and associated with an abnormal inflammatory response of the lungs to noxious particles or gases. Chronic obstructive pulmonary disease (COPD) is an umbrella term for a group of respiratory tract diseases that are characterized by airflow obstruction or limitation. Conditions included in this umbrella term are: chronic bronchitis, emphysema, and bronchiectasis.

In another embodiment, the nanoparticles are used for the treatment of Asthma. Also, the nanoparticles are used for the treatment of Endotoxemia and sepsis. In one embodiment, the nanoparticles are used to for the treatment of rheumatoid arthritis (RA). In another embodiment, the nanoparticles are used for the treatment of Psoriasis. In yet another embodiment, the nanoparticles are used for the treatment of contact or atopic dermatitis. Contact dermatitis includes irritant dermatitis, phototoxic dermatitis, allergic dermatitis, photoallergic dermatitis, contact urticaria, systemic contact-type dermatitis and the like. Irritant dermatitis can occur when too much of a substance is used on the skin of when the skin is sensitive to certain substance. Atopic dermatitis, sometimes called eczema, is a kind of dermatitis, an atopic skin disease.

Further, the nanoparticles may be used for the treatment of Glomerulonephritis. Additionally, the nanoparticles may be used for the treatment of Bursitis, Lupus, Acute disseminated encephalomyelitis (ADEM), Addison's disease, Antiphospholipid antibody syndrome (APS), Aplastic anemia, Autoimmune hepatitis, Coeliac disease, Crohn's disease, Diabetes mellitus (type 1), Goodpasture's syndrome, Graves' disease, Guillain-Barré syndrome (GBS), Hashimoto's disease, inflammatory bowel disease, Lupus erythematosus, Myasthenia gravis, Opsoclonus myoclonus syndrome (OMS), Optic neuritis, Ord's thyroiditis, ostheoarthritis, uveoretinitis, Pemphigus, Polyarthritis, Primary biliary cirrhosis, Reiter's syndrome, Takayasu's arteritis, Temporal arteritis, Warm autoimmune hemolytic anemia, Wegener's granulomatosis, Alopecia universalis, Chagas' disease, Chronic fatigue syndrome, Dysautonomia, Endometriosis, Hidradenitis suppurativa, Interstitial cystitis, Neuromyotonia, Sarcoidosis, Scleroderma, Ulcerative colitis, Vitiligo, Vulvodynia, Appendicitis, Arteritis, Arthritis, Blepharitis, Bronchiolitis, Bronchitis, Cervicitis, Cholangitis, Cholecystitis, Chorioamnionitis, Colitis, Conjunctivitis, Cystitis, Dacryoadenitis, Dermatomyositis, Endocarditis, Endometritis, Enteritis, Enterocolitis, Epicondylitis, Epididymitis, Fasciitis, Fibrositis, Gastritis, Gastroenteritis, Gingivitis, Hepatitis, Hidradenitis, Ileitis, Iritis, Laryngitis, Mastitis, Meningitis, Myelitis, Myocarditis, Myositis, Nephritis, Omphalitis, Oophoritis, Orchitis, Osteitis, Otitis, Pancreatitis, Parotitis, Pericarditis, Peritonitis, Pharyngitis, Pleuritis, Phlebitis, Pneumonitis, Proctitis, Prostatitis, Pyelonephritis, Rhinitis, Salpingitis, Sinusitis, Stomatitis, Synovitis, Tendonitis, Tonsillitis, Uveitis, Vaginitis, Vasculitis, or Vulvitis.

In some embodiments, the nanoparticles may be used for the treatment of cancers. In some embodiments, the invention provides a method of treating breast cancer such as a ductal carcinoma in duct tissue in a mammary gland, medullary carcinomas, colloid carcinomas, tubular carcinomas, and inflammatory breast cancer. In some embodiments, the invention provides a method of treating ovarian cancer, including epithelial ovarian tumors such as adenocarcinoma in the ovary and an adenocarcinoma that has migrated from the ovary into the abdominal cavity. In some embodiments, the invention provides a method of treating cervical cancers such as adenocarcinoma in the cervix epithelial including squamous cell carcinoma and adenocarcinomas. Similarly the invention provides methods to treat prostate cancer, such as a prostate cancer selected from the following: an adenocarcinoma or an adenocarinoma that has migrated to the bone. Similarly the invention provides methods of treating pancreatic cancer such as epitheliod carcinoma in the pancreatic duct tissue and an adenocarcinoma in a pancreatic duct. Similarly the invention provides methods of treating bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers. Similarly, the invention provides methods of treating acute myeloid leukemia (AML), preferably acute promyleocytic leukemia in peripheral blood. Similarly the invention provides methods to treat lung cancer such as non-small cell lung cancer (NSCLC), which is divided into squamous cell carcinomas, adenocarcinomas, and large cell undifferentiated carcinomas, and small cell lung cancer. Similarly the invention provides methods to treat skin cancer such as basal cell carcinoma, melanoma, squamous cell carcinoma and actinic keratosis, which is a skin condition that sometimes develops into squamous cell carcinoma. Similarly the invention provides methods to treat eye retinoblastoma. Similarly the invention provides methods to treat intraocular (eye) melanoma. Similarly the invention provides methods to treat primary liver cancer (cancer that begins in the liver). Similarly, the invention provides methods to treat kidney cancer. In another aspect, the invention provides methods to treat thyroid cancer such as papillary, follicular, medullary and anaplastic. Similarly the invention provides methods to treat AIDS-related lymphoma such as diffuse large B-cell lymphoma, B-cell immunoblastic lymphoma and small non-cleaved cell lymphoma. Similarly the invention provides methods to treat Kaposi's sarcoma. Similarly the invention provides methods to treat viral-induced cancers. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. Similarly the invention provides methods to treat central nervous system cancers such as primary brain tumor, which includes gliomas (astrocytoma, anaplastic astrocytoma, or glioblastoma multiforme), Oligodendroglioma, Ependymoma, Meningioma, Lymphoma, Schwannoma, and Medulloblastoma. Similarly the invention provides methods to treat peripheral nervous system (PNS) cancers such as acoustic neuromas and malignant peripheral nerve sheath tumor (MPNST) including neurofibromas and schwannomas. Similarly the invention provides methods to treat oral cavity and oropharyngeal cancer. Similarly the invention provides methods to treat stomach cancer such as lymphomas, gastric stromal tumors, and carcinoid tumors. Similarly the invention provides methods to treat testicular cancer such as germ cell tumors (GCTs), which include seminomas and nonseminomas; and gonadal stromal tumors, which include Leydig cell tumors and Sertoli cell tumors. Similarly the invention provides methods to treat testicular cancer such as thymus cancer, such as to thymomas, thymic carcinomas, Hodgkin disease, non-Hodgkin lymphomas carcinoids or carcinoid tumors.

In some embodiments, the invention can be used to treat bone cancers, such as osteosarcoma. Osteosarcoma is the most common primary malignant neoplasm of bone in children and adolescents and is characterized by a clonal unregulated proliferation of primitive osteoid-producing mesenchymal cells. The development of neoadjuvant cytotoxic chemotherapy regimens over the past three decades, has dramatically improved the fate of osteosarcoma patients. The addition of multi-agent regimens plus refinement in surgical resection has resulted in a 65-75% long term survival rate in patients presenting with localized disease (Gurney et al., 1999 Malignant bone tumors. In: Cancer Incidence and Survival Among Children and Adolescents: United States SEER Program 1975-1995). While this is a substantial improvement, current multi-modality therapy still has significant shortcomings. First, the outlook remains poor for patients with overt metastases at diagnosis or for those in whom the cancer recurs. Second, while the currently utilized chemotherapy regimens are effective against osteosarcoma, they also wreak havoc on normal cells that can result in acute and potentially life-threatening complications. It is also now appreciated that exposure of pediatric cancer patients to cytotoxic chemotherapy can lead to secondary malignancies and other medical maladies, decades after their tumor has been eradicated. In some embodiments, targeted liposomal nanoparticles can be used to deliver chemotherapy or other types of agents selectively to tumors with reduced side effects on normal cells. In some embodiments, targeted nanoparticles can bind to tumors that are clinically undetectable.

Kits

The invention also provides kits. The kits include the nanoparticles described herein, in suitable packaging, and written material that can include instructions for use, discussion of clinical studies, listing of side effects, and the like. Suitable packaging and additional articles for use (e.g., measuring cup for liquid preparations, foil wrapping to minimize exposure to air, and the like) are known in the art and may be included in the kit.

The nanoparticles may be provided dry or in a storage solution, and may be pre-polymerized or polymerized before administration, e.g. by UV light exposure. The nanoparticle solutions may be ready for administration immediately, or may be suspended or mixed with additional compounds or solutions before administration. The nanoparticles provided may already contain therapeutic, targeting, or contrast agents for usage, or such agents may be linked or incorporated into the nanoparticles on-site. Nanoparticles may further be provided in specific sizes for different routes of administration, or may be comprised of a heterogeneous distribution of sizes.

The reagents may also include ancillary agents such as buffering agents and stabilizing agents, e.g., polysaccharides and the like. The kit may further include, where necessary, agents for reducing background interference in a test, control reagents, apparatus for conducting a test, and the like. The kit may be packaged in any suitable manner, typically with all elements in a single container along with a sheet of printed instructions for carrying out the test.

Such kits can enable the detection of the nanoparticles, which are suitable for the clinical detection, prognosis, and screening of cells and tissue from patients, such as the conditions described herein.

Such kits may additionally comprise one or more therapeutic agents. The kit may further comprise a software package for data analysis, which may include reference date for comparison with the test results.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer.

This invention is further illustrated by the following examples which should not be construed as limiting. The contents of all references cited throughout this application, as well as the figures and table are incorporated herein by reference.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### EXAMPLES

### Example 1

### Binding of HPLNs to ALCAM-Expressing Osteosarcoma Cell Lines

Osteosarcoma is the most common primary malignant neoplasm of bone in children and adolescents and is characterized by a clonal unregulated proliferation of primitive osteoid-producing mesenchymal cells (Huvos A G, 1991, Bone tumors: diagnosis, treatment, and prognosis, pp viii, 784 p). Prior to 1970, the prognosis for patients with osteosarcoma who were treated with surgery alone, was a dismal 10-20% overall survival. Though aggressive surgeries would render most patients grossly tumor free, the vast majority would develop progressively fatal metastatic disease within two years. This suggested that at the time of their initial diagnosis, clinically undetectable tumor had already spread to distant sites in most patients and that effective systemic anticancer therapy was needed (Dahlin et al., 1997, Osteosarcoma of bone and its important recognizable varieties, American Journal of Surgical Pathology).

The development of neoadjuvant cytotoxic chemotherapy regimens over the past three decades, has dramatically improved the fate of osteosarcoma patients. The addition of multi-agent regimens plus refinement in surgical resection has resulted in a 65-75% long term survival rate in patients presenting with localized disease (Gurney J G S A, et al., 1999 Malignant bone tumors. In: Cancer Incidence and Survival Among Children and Adolescents: United States SEER Program 1975-1995. SEER program, National Cancer Institute, Bethesda, Md., pp 99-110). While this is a substantial improvement, current multi-modality therapy still has significant shortcomings. First, the outlook remains poor for patients with overt metastases at diagnosis or for those in whom the cancer recurs. Second, while the currently utilized chemotherapy regimens are effective against osteosarcoma, they also wreak havoc on normal cells that can result in acute and potentially life-threatening complications. It is also now appreciated that exposure of pediatric cancer patients to cytotoxic chemotherapy can lead to secondary malignancies and other medical maladies, decades after their tumor has been eradicated (Janeway K A, Grier H E, 2010, Sequelae of osteosarcoma medical therapy: a review of rare acute toxicities and late effects. Lancet Oncol 11:670-678).

With these criteria in mind, the cell surface receptor ALCAM (Activated Leukocyte Adhesion Molecule, CD-166) is an attractive candidate to target osteosarcoma. This glycoprotein is a member of the immunoglobulin superfamily and is thought to mediate important cell-cell interactions involved in cell migration, neurogenesis, hematopoiesis and the immune response (Swart G W, 2002, Activated leukocyte cell adhesion molecule (CD166/ALCAM): developmental and mechanistic aspects of cell clustering and cell migration. Eur J Cell Biol 81:313-321). More recently increased ALCAM expression has been linked to a variety of cancers including pancreatic, breast, prostate, and colorectal carcinomas and melanoma (Ofori-Acquah S F, King J A, 2008, Activated leukocyte cell adhesion molecule: a new paradox in cancer, Transl Res; Kristiansen G, et al., 2003, ALCAM/CD166 is up-regulated in low-grade prostate cancer and progressively lost in high-grade lesions, Prostate; King J A, et al., 2004, Activated leukocyte cell adhesion molecule in breast cancer: prognostic indicator, Breast Cancer Res). Furthermore, others have found that immunoliposomes coated with a recombinant anti-ALCAM monoclonal antibody were taken up by prostate cancer cell lines expressing this antigen (Liu B, et al., 2007, Anti-CD166 single chain antibody-mediated intracellular delivery of liposomal drugs to prostate cancer cells, Molecular Cancer Therapeutics).

In this example, we demonstrate that ALCAM is overexpressed in both osteosarcoma tumor derived cell lines and primary biopsy specimens. We show that this cell surface molecule can be exploited to enhance binding and uptake of nanoparticles by osteosarcoma cells. We present a new, polymerized liposome formulation consisting of a mixture of lipids with saturated and diacetylene containing acyl chains.

### Methods

Materials.

Conventional and Hybrid Polymerized Liposomal Nanoparticles (HPLNs) were obtained from NanoValent Pharmaceuticals, Inc. (Bozeman, Mont.). The components comprising the conventional liposomes are L-α-phosphatidylcholine hydrogenated soy, (“hydrogenated soy PC”), cholesterol and 1,2-distearoyl-sn-glycero-3-phosphoethanolamine-N-[methoxy(polyethylene glycol)-2000] (“m-Peg₂₀₀₀-DSPE”), (Avanti Polar Lipids, Alabaster, Ala.). The HPLNs are comprised of: N-(5′-hydroxy-3′-oxypentyl)-10-12-pentacosadiynamide (“h-Peg₁-PCDA”), hydrogenated soy PC, m-Peg₂₀₀₀-DSPE, 1,2-distearoyl-sn-glycero-3-phosphoethanolamine-N-[maleimide(polyethylene glycol)-2000] (“mal-Peg₂₀₀₀-DSPE”) and cholesterol.

### Preparation of Conventional Liposomes and HPLNs

Conventional liposomes were prepared from hydrogenated soy PC, cholesterol and m-PEG₂₀₀₀-DSPE in molar proportions of 57.5:37.5:5, non-targetable HPLNs prepared from h-PEG₁PCDA, hydrogenated soy PC, cholesterol and m-PEG₂₀₀₀-DSPE at a molar proportion of 15:47:32:6, and targetable HPLNs prepared from h-PEG₁PCDA, hydrogenated soy PC, cholesterol, mal-PEG₂₀₀₀-DSPE and m-PEG₂₀₀₀-DSPE at a molar proportion of 15:47:32:4.5:1.5, according to the method previously described (R. E. Bruehl, F. Dasgupta, T. Katsumoto, J. H. Tan, C. R. Bertozzi, W. Spevak, D. J. Ahn, S. D. Rosen, J. O. Nagy, “Polymerized Liposome Assemblies: Bifunctional Macromolecular Selectin Inhibitors Mimicking Physiological Selectin Ligands”, Biochem., 2001, 40, 5964-5974.). Briefly, lipids were mixed and evaporated in vacuo, to a film. Deionized water or 300 mM ammonium sulfate was added to the films so as to give a 25 mM (total lipid and cholesterol) suspension. The suspension was heated via sonication between 70-80° C. with a probe-tip sonicator (Fisher sonic dismembrator model 300) for 5 min. The resulting milky solution was then passed through a stacked polycarbonate membrane (100 nm), eleven times, with a dual syringe extruder (LiposoFast-Basic, Avestin, Inc., Ottawa, ON, Canada), heated to 65° C. The nearly clear liposome solutions were cooled to 5° C. for 12 hours. After warming to ambient temperature, the water-filled liposomes that contain PCDA lipids were polymerized by UV light irradiation (254 nm) with a Spectrolinker XL-1000 UV Crosslinker (Spectronics Corp.) for 10 minutes. The resulting blue HPLNs were heated to 65° C. for 5 min to convert them to the red (fluorescent) form. The colored solutions were syringe filtered through 0.2 μm cellulose acetate filters in order to remove trace insoluble contaminants.

### Doxorubicin Loading

The ammonium sulfate-containing conventional and polymerizable liposomes were passed over a G50 Sephadex column (washed with 20 mM HEPES) to exchange the external buffer. The liposomes were then incubated with doxorubicin-hydrochloride (Shandong Tianyu Fine Chemical Co., Ltd.) in a ratio of 1 μM of doxorubicin to 3.2 μM of lipid while heating to 65° C. for 20 min. The unencapsulated doxorubicin was removed by shaking with anionic exchange resin (BioRex 70, BibRad Inc.) in a ratio of 7 μg of doxorubicin to 1 μl of packed resin, for 5 min. Liposomes were separated from resin by filtering through Pierce Spin Cups. The average particle size measurements were obtained on a Zetasizer Nano-S (Malvern Inst.), in a solution of 10 mM sodium chloride.

### Preparation of ALCAM Antibody Conjugated HPLNs

An anti-ALCAM antibody was previously engineered into a cys-diabody (cross-paired dimer of single-chain antibody fragments, with C-terminal cysteine residues) as described by Liu et al. See Liu B., et al. (2007) J. Mol. Med. (Berl) 85:1113-1123, which is herein incorporated by reference in its entirety. Liposomes were loaded with dox, and then anti-ALCAM cys-diabody was conjugated to the particle surface. TCEP (500 mM) was added to cys-diabody (1-4 μg/uL) solution to a final concentration of 10 mM and incubated at room temperature for 30 minutes to reduce the diabody's terminal cysteine residues. Reduced α-ALCAM cys-diabody was then added to the liposome mixture (2.5-10 μg/uL lipid) at a diabody/lipid ratio of 1 μg diabody:7.5 μg total lipid and incubated at room temperature for 2 hours to allow for conjugation to Maleimide residues on PLN. Unbound maleimide residues were quenched with 20 mM cysteine solution for 30 minutes. Unbound diabody, free cysteine and TCEP were removed using filtration through Amicon Ultra-0.5 mL 100K centrifugal filters (Millipore). Samples were diluted 1:2 with HEPES buffered saline and centrifuged at 6000 rpm for 10 minutes to concentrate the Dox-loaded ALCAM diabody conjugated sample.

### Quantification of Entrapped Liposomal Doxorubicin

Doxorubicin was quantified spectrophotometrically based on the molar extinction coefficient of 12,500. Unencapsulated Dox was removed using Bio-Rex 70. Dox-loaded particles were disrupted using diluted a 1:20 Isopropanol with 0.075 mM HCl solution and then vortexed for at least 30 seconds to ensure complete membrane rupture. Absorbance was read at 480 nm on a Beckman Coulter. DU800 spectrophotometer.

### Quantification of Total Lipid

Total lipid content of PLN samples was measured using a colorimetric assay. A 4 μL aliquot of PLN sample was vacuum dried and resuspended in an ammonium ferrothiocyanate/chloroform solution then centrifuged at 14,000 rpm for 2 minutes. Absorbance at 488 nm of the organic phase was then measured in a Beckman Coulter DU800 spectrophotometer. OD488 of the sample was then compared to a standard curve of known lipid concentration values.

### MTT Assay

Osteosarcoma cell lines KHOS 240S, HOS or MNNG-HOS were grown in Dulbecco's Modified Eagle Medium (HyClone Cat #SH30022.01) with 10% fetal bovine serum (Gemini Bioproducts). Cells were seeded in a 96-well format at a concentration of 5×10³ cells/well at a volume of 100 μL media with penicillin/streptomycin and incubated overnight. The following day, wells were treated with doxorubicin loaded targeted PLNs, untargeted PLNs, conventional liposomes or free doxorubicin for a four hour period then washed with fresh media. Doses were added based on doxorubicin concentrations ranging on a log scale from 0.01 to 100 μM and at 0 nM. The 0 nM well was treated with HEPES buffered saline. Each treatment was performed in triplicate. Cells were incubated under standard CO₂ conditions for 72 hrs at 37° C. At 72 hrs, all wells are treated with 10 μL of thiazolyl blue tetrazolium bromide (Sigma) solution at an initial concentration of 5 in phosphate buffered saline and incubated for 4 hrs. Reaction was ceased and cells lysed by adding 100 μL of 15% sodium dodecyl sulfate/15 mM HCl solution and incubated overnight in the dark at room temperature. Plate absorbance was read using Bio-rad microplate reader at 570 nm. To account for background absorbance, the arithmetic mean of the OD570 of the blank wells was subtracted from the OD570 readings of all treated wells. The arithmetic mean of each plate was calculated and considered as 100% viability. The remaining wells were then divided by this mean to obtain nominal percent viability within each well. Viability was plotted against log drug concentration, and unweighted nonlinear regression was used to estimate log(IC50) for each treatment using a four-parameter sigmoid dose-response model (Prism Software, Graphpad). Fixing the bottom parameter to zero yielded better residual patterns and more stable Hill slope estimates than analyses allowing a variable bottom. For each cell line experiment, a run comparing the four treatment vehicles was repeated 3 to 7 times on different days. Within each cell line, a linear mixed effects model revealed day-to-day variability as a much greater source of variation in log(IC50) than batch variability, and blocking on experiment day improved the precision of estimated differences between treatments. In assessing IC50 results across cell lines, a significant cell line by treatment interaction was detected that could be fully accounted for by modeling a shift in conventional liposome potency (relative to the other 3 treatments) just in the MNNG-HOS cell line.

### PLN Binding Fluorescent Microscopy Assay

Osteosarcoma cell lines were seeded onto 4-well Lab-tek II Chamber Slides (Thermo Scientific) to reach 80% confluence overnight. Cells were treated with anti-ALCAM diabody conjugated PLN at 50 μg/mL per well. Cells were incubated for 4 hrs at 37° C. Media was removed and wells were washed with 1 mL fresh media. Cell fixation was with 3.7% formaldehyde in Phosphate buffered saline for 15 minutes at 4° C. Cells were mounted using VectaShield mounting medium with DAPI (Vector Laboratories). Positive and negative control cell lines were pancreatic cell lines HPAF and MiaPaca, respectively. Cells were viewed using a Carl Zeiss AxioImager DI fluorescence microscope. Cells were viewed at 20× magnification. DAPI was visualized through blue/cyan filter. Bound nanoparticles were visualized using the Rhodamine filter at a 1 second exposure.

### Western Immunoblot

Antibodies used for immunoblot were monoclonal mouse anti-CD166 (Vector Laboratories, Cat# VP-C375) at a concentration of 1:400 and anti-Actin C-11 (Santa Cruz Biotechnology, Cat#sc-1615) at a concentration of 1:3000.

### Immunohistochemistry

De-identified human patient osteosarcoma paraffin-embedded samples were obtained from the UCLA Tissue Procurement Core Laboratory (IRB Exempt). Four-micrometer sections were cut and placed onto slides. Samples were then deparaffinized, rehydrated, and subjected to heat-induced epitope retrieval. Slides were incubated with a 1:50 dilution of anti-CD166 mouse monoclonal antibody (Vector) for 2 h at room temperature, and signal was detected using the mouse EnVision+ System-HRP (DAB) kit (Dako). Sections were counterstained with hematoxylin. Images were viewed and obtained using Zeiss AxioImager at 20× magnification.

### Results

ALCAM is Highly Expressed in Both Primary Osteosarcoma Specimens and Tumor Derived Cell Lines.

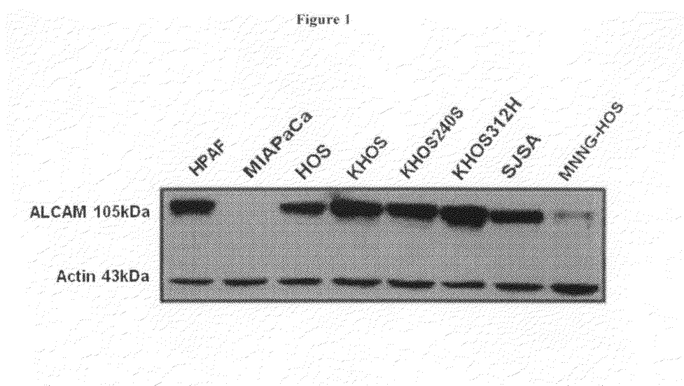

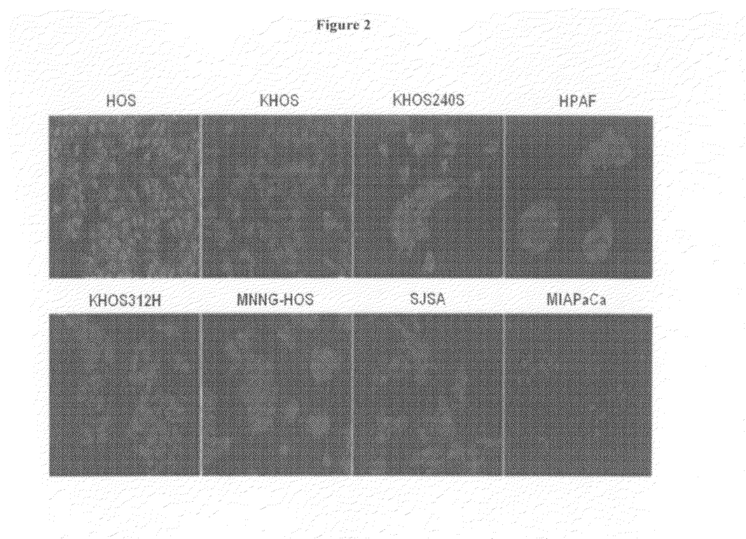

A molecular survey of the osteosarcoma cell line U2-OS, demonstrated expression of ALCAM on the surface of these cells (Nelissen J M, et al., 2000, Molecular analysis of the hematopoiesis supporting osteoblastic cell line U2-OS. Exp Hematol). These observations prompted a more in depth investigation of ALCAM expression in human osteosarcoma. Evaluation of ALCAM expression in a collection of 6 tumor-derived cell lines was used as an initial platform. Cell lysates were harvested from subconfluent adherent cultures grown in tissue culture and analyzed by immunoblot using anti-ALCAM antisera. Pancreatic cancer cell lines with high (HPAF) and no (MiaPaCa) ALCAM expression were used as controls. All 6 osteosarcoma cell lines expressed ALCAM and 5 of 6 demonstrated elevated expression at the level seen in the HPAF control (FIG. 1). The quality of ALCAM expression was further confirmed in fluorescent immunohistochemistry showing primarily a membranous, surface component to the ALCAM expression in osteosarcoma cell lines (FIG. 2).

Though there was a high frequency of ALCAM expression in our cell line collection, there is always concern that it may be due to a selection process inherent in creating tumor derived cell lines. In addition, differences in growth conditions between in vivo in osteosarcoma patients and in vitro in tissue culture, may be responsible for changes in ALCAM expression.

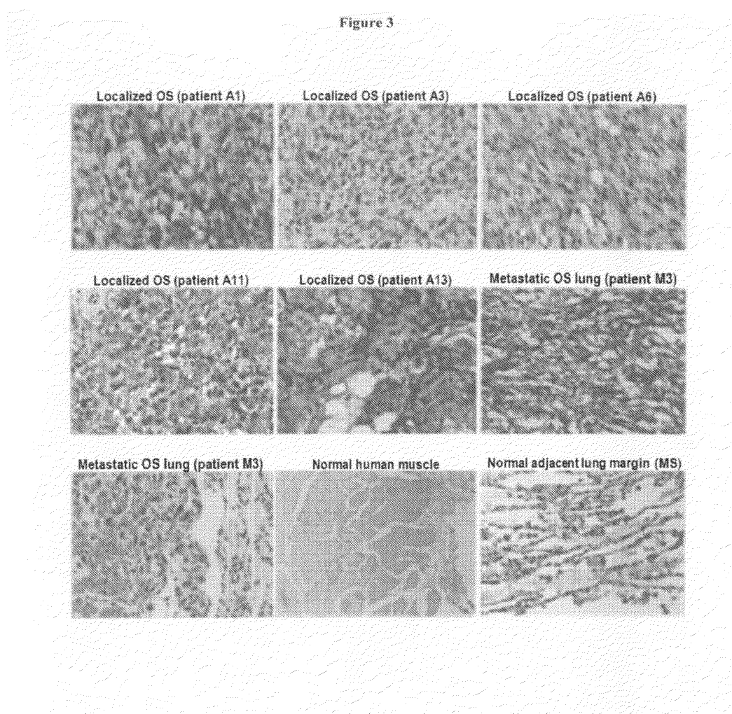

To address these concerns, human osteosarcoma tumor samples both from primary and metastatic sites were assayed for ALCAM expression by immunocytochemistry. Banked anonymized patient specimens were fixed, sectioned and incubated with anti-ALCAM antisera. After washing, in situ ALCAM expression was detected using a colorimetric assay and evaluated by light microscopy. Tissues were graded as strongly positive (+++), moderately positive (++), weakly positive (+) or negative (−). All OS tumor samples stained positively for ALCAM. Of 10 localized and metastatic OS samples 5 of the localized OS tissues stained weakly to strongly positive for ALCAM and 5 of the metastatic OS samples also had moderate to strong IHC staining. Osteosarcoma cells demonstrated both cytoplasmic and membranous ALCAM expression (Representative IHC images are shown in FIG. 3).

### Anti-ALCAM Coupled Hybrid Polymerized Liposomal Nanoparticles Avidly Bind to Osteosarcoma Cell Lines.

Hybrid polymerized liposomal nanoparticles (HPLNs) were evaluated as a potential therapeutic delivery vehicle that could be targeted to osteosarcoma cells expressing ALCAM. HPLNs share many structural attributes of conventional liposomes. They are self-assembling unilamellar spheres whose surfaces can be modified using the same chemical coupling strategies as employed for liposomes. Unlike liposomes, HPLNs can be manufactured to be intrinsically fluorescent. Ultraviolet irradiation leads to cross-linking of diacetylene residues present in their acyl chains, leading to highly colored blue particles and heat treatment of the HPLN vesicles results in a color change and fluorophore formation (Eckhardt, H.; Boudreaux, D. S.; Chance, R. R. J. Chem. Phys. 1986, 85, 4116, J I Olmsted and M Strand J. Phys. Chem. 1983, 87, 4790). The fluorescence emission spectrum is centered at 635 nm with a broad and complex excitation spectrum from 480-580 nm. As a result, HPLNs converted into their fluorescent form can be readily traced from the time they bind to target cells until they are deposited and compartmentalized into sub-cellular structures.

Targeted HPLNs were created by chemically coupling a recombinant anti-ALCAM monoclonal antibody to its surface. HPLNs were synthesized containing maleimide reactive groups at the distal end of surface polyethylene glycol (PEG) molecules. A bivalent anti-ALCAM diabody derived from the previously described ScFv, was genetically engineered to contain a C-terminal cysteine (McCabe K E, et al., 2011, An Engineered Cysteine-Modified Diabody for Imaging Activated Leukocyte Cell Adhesion Molecule (ALCAM)-Positive Tumors. Mol Imaging Biol). Mixing these two components induced a condensation reaction between the thiol of the cysteine and the maleimide moiety, resulting in the anti-ALCAM diabody being covalently coupled to the HPLN surface. As a negative control, untargeted HPLNs were made in the same manner by coupling free cysteine to nanoparticles.

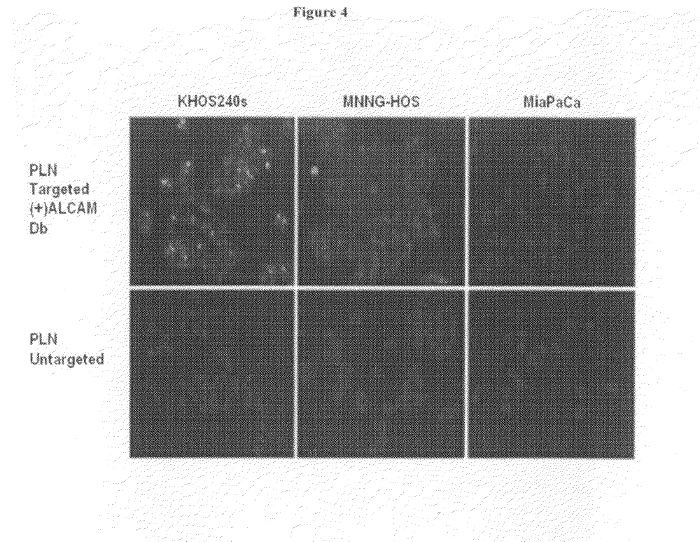

Binding studies were performed comparing the relative affinities of anti-ALCAM coupled HPLNs (α-AL-HPLN) versus untargeted PLNs towards osteosarcoma cell lines. After a 4 hour incubation, cells were washed and α-AL-HPLN binding was detected by fluorescence microscopy. α-AL-HPLNs bound to all of the osteosarcoma cell lines in our panel, much more efficiently than untargeted negative controls (FIG. 4). This interaction was dependent on cellular ALCAM expression. Both targeted and untargeted HPLNs bound equally to MiaPaCa cells that do not express cell surface ALCAM.

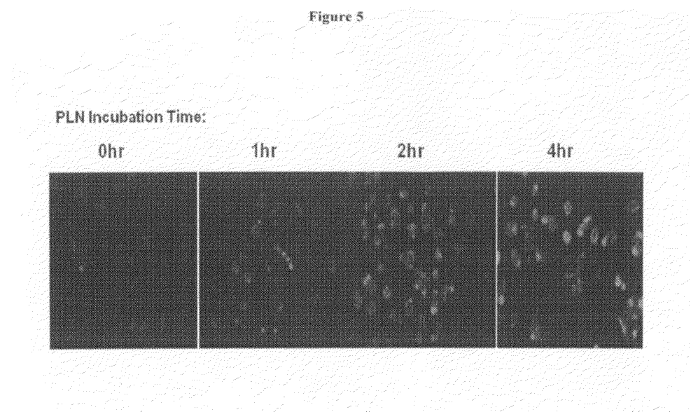

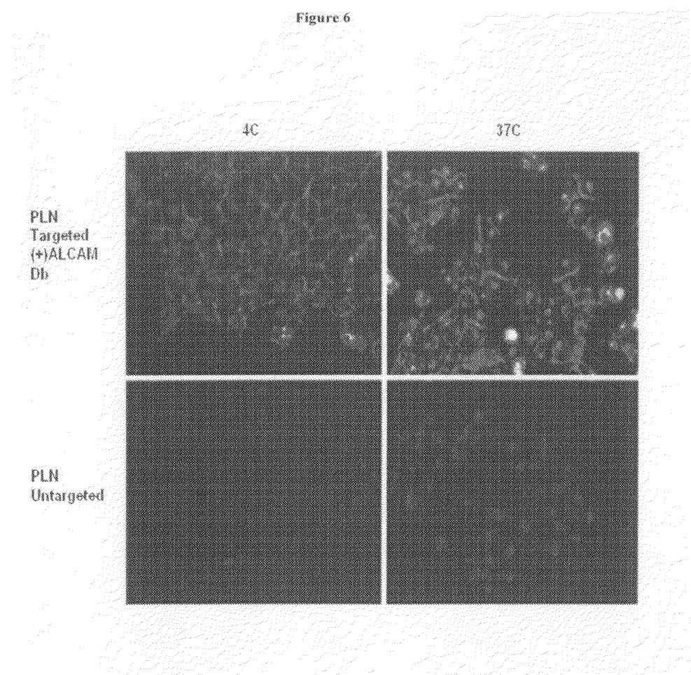

To gauge the rapidity of the interaction between α-AL-HPLNs and osteosarcoma cells, a time course study was performed. Osteosarcoma cells were incubated with α-AL-HPLNs for varying time periods up to 4 hours, washed and then evaluated by fluorescence microscopy. α-AL-HPLN binding was detected as early as 30 minutes and reached a maximum by 4 hours (FIG. 5). The presence of a strong perinuclear fluorescence signal suggested that the targeted nanoparticles were rapidly internalized into the endosome compartment of the cell. To further evaluate this, binding studies were performed at 4° C. which would inhibit cellular endocytosis. Under these conditions, a strong membrane fluorescence signal was detected without perinuclear nuclear localization, consistent with α-AL-HPLNs being bound to the cell surface but not internalized (FIG. 6).

### Discussion

Our data clearly demonstrate an increase in ALCAM expression in osteosarcoma, though the biologic consequences of this are difficult to gauge. The normal physiologic roles of ALCAM are still coming to light but its molecular structure and clustering at tight junctions suggests that it could be involved in cell adhesion and migration (Bowen M A, Aruffo A, 1999, Adhesion molecules, their receptors, and their regulation: Analysis of CD6-activated leukocyte cell adhesion molecule (ALCAM/CD166) interactions, Transplantation Proceedings 31:795-796). In this context, it is tempting to think that modulating ALCAM expression could potentiate the invasive and metastatic behaviors found in high-grade malignancies such as osteosarcoma. However there is no consistent correlation between ALCAM expression level and patient survival across all cancers. For example, an increase in ALCAM expression is found in higher stage, more aggressive malignant melanoma (van Kempen L C L T, van den Oord J J, van Muijen G N P, Weidle U H, Bloemers H P J, Swart G W M 2000 Activated leukocyte cell adhesion molecule/CD166, a marker of tumor progression in primary malignant melanoma of the skin. American Journal of Pathology 156:769-774). By contrast high ALCAM is correlated with low grade, leis aggressive cases of prostate cancer (Kristiansen G, et al., 2003). Considering the high frequency of elevated ALCAM expression in even our small cohort of osteosarcomas, it may not be able to discriminate between high and low risk patients with this disease.

Though ALCAM may be a limited prognostic biomarker in osteosarcoma, it has potential to serve as a molecule through which to therapeutically target this tumor. Fluorescent nanoparticles coated with anti-ALCAM diabodies preferentially bind to osteosarcoma cell lines, even those that express ALCAM at relatively low levels (data not shown). As seen in prostate cancer cells, ALCAM targeted nanoparticles were rapidly internalized by osteosarcoma cells suggesting a strategy for intracellular delivery of anti-cancer agents.

The use of diacetylene containing lipids to create polymerizable films and vesicles has been intensively studied for creating biosensors (Cabral E C M, et al., 2003, Preparation and characterization of diacetylene polymerized liposomes for detection of autoantibodies, Journal of Liposome Research 13:199-211; and M A Reppy, B A Pindzola, Biosensing with polydiacetylene materials, structure, optical properties and applications. Chem. Commun. 2007, 4317-4338) and have been explored as cancer diagnostic and delivery vehicles (Guo C X, et al., 2010, Polydiacetylene vesicles as a novel drug sustained-release system, Colloids and Surfaces B-Biointerfaces 76:362-365; Puri A, et al., 2011, A novel class of photo-triggerable liposomes containing DPPC:DC(8,9)PC as vehicles for delivery of doxorubicin to cells, Biochimica Et Biophysica Acta-Biomembranes 1808:117-126; Li Z, et al., 2011, Partially polymerized liposomes: stable against leakage yet capable of instantaneous release for remote controlled drug delivery, Nanotechnology 22; Sipkins D A, Cheresh D A, Kazemi M R, Nevin L M, Bednarski M D, Li K C. (1998) Detection of tumor angiogenesis in vivo by alphaVbeta3-targeted magnetic resonance imaging. Nat Med 4(5): 623-6; Li L, Wartchow C A, Danthi S N, Shen Z, Dechene N, Pease J, Choi H S, Doede T, Chu P, Ning S, Lee D Y, Bednarski M D, Knox S J. (2004) A novel antiangiogenesis therapy using an integrin antagonist or anti-Flk-1 antibody coated ⁹⁰Y-labeled nanoparticles. Int J Radiat Oncol Biol Phys 58(4): 1215-27; Kien Vuu, Jianwu Xie, Michael A. McDonald, Marcelino Bernardo, Finie Hunter, Yantian Zhang, King Li, Mark Bednarski, and Samira Guccione, Gadolinium-Rhodamine Nanoparticles for Cell Labeling and Tracking via Magnetic Resonance and Optical Imaging Bioconjugate Chem. 2005, 16, 995-999; and AMICHAI YAVLOVICH, BRANDONSMITH, KSHITIJ GUPTA, ROBERT BLUMENTHAL, & ANU PURI, Light-sensitive lipid-based nanoparticles for drug delivery: design principles and future considerations for biological applications Molecular Membrane Biology, October 2010; 27(7): 364-381) When these membranes are treated with ultraviolet irradiation the resulting intralipid cross-links form an intensely blue chromophore. When exposed to physiochemical perturbations such as heat, shear or pH stress, these membranes shift from a blue non-fluorescent state to a red fluorescent state (as cited earlier: Eckhardt, H.; Boudreaux, D. S.; Chance, R. R. J. Chem. Phys. 1986, 85, 4116, J I Olmsted and M Strand J. Phys. Chem. 1983, 87, 4790). A distinct advantage to the PLN fluorescence is that little or no photobleaching occurs. Taking advantage of these properties, we were able to track binding and internalization of red, fluorescent ALCAM targeted PLNs (α-AL-PLN) that had been treated with UV irradiation and heat. Interestingly, we obtained the same results using a similar preparation of α-AL-PLN that received only UV irradiation and were therefore blue and non-fluorescent in solution (data not shown). It appears that the interaction between the coupled diabody molecules and the cell surface ALCAM proteins exerted sufficient stress to shift the bound α-AL-PLN into a fluorescent state.

### Example 2

### Formulating HPLNs as Potential Therapeutic Delivery Vehicles

In this example, we show that HPLNs, when loaded with doxorubicin, display enhanced cytotoxicity to osteosarcoma cells. Our initial PLN formulation was composed entirely of 10,12-pentacosadiynoic acid (PCDA) derivatives and when polymerized formed a very fluorescent particle that could easily be detected. However these nanoparticles proved problematic when trying to adapt them for delivery of therapeutics. Attempts at effectively loading them with cytotoxic chemotherapeutic agents, either through encapsulation during vesicle formation or across ion gradients using the prepolymerized liposomes, failed at multiple levels. For this reason, hybrid PLNs were created that were composed of PCDA lipids mixed with saturated phospholipids found in many liposome formulations.

To approach this problem, we started with a standard liposomal formulation consisting of hydrogenated soy PC (where the major component is distearoylphosphatidyl-choline (DSPC)), cholesterol and polyethylene glycol-distearoylphosphatidyl-ethanolamine (m-PEG₂₀₀₀-DSPE) in molar proportions of 57.5:37.5:5. Increasing amounts of unsaturated PCDA lipids were then added. We chose a very short Peg chain PCDA derivative, h-Peg1-PCDA, because it is an extremely reactive crosslinking lipid, has good aqueous dispersion properties when mixed with charged lipids, is in itself uncharged so it won't alter the overall surface charge, and the small polar head won't interfere with the conjugation of targeting agents. After sonication and extrusion, vesicles were evaluated for size by dynamic light scattering and the ability to form fluorescent particles when treated with UV irradiation and heat. We found that inclusion of as little as 15 mole % h-Peg₁-PCDA resulted in brightly fluorescent particles but that this signal became progressively attenuated with decreasing h-Peg₁-PCDA proportions.

Considering that our HPLNs were a heterogeneous mix of lipids with two very different acyl chain structures, stability in solution was a major concern. Certain hybrid formulations formed insoluble aggregates within hours after extrusion. Overnight cooling at 10° C. immediately after extrusion, but prior to polymerization, proved critical in creating stable HPLNs. Particles treated this way were stable for weeks either refrigerated or at room temperature. HPLNs of the same lipid composition constructed without this cooling step, were irretrievably unstable. Evidence found in published studies with similar mixtures of longer chain diacetylene lipids and shorter chain phosphotidyl-choline lipids suggest that a phase separation occurs between the lipid types (Moran-Mirabal J A, Aubrecht D A, Craighead H G 2007 Phase separation and fractal domain formation in Phospholipid/Diacetylene-Supported lipid bilayers. Langmuir 23:10661-10671; and Gaboriaud, R. Volinsky, A. Berman, R. Jelinek: J. Colloid Interface Sci. 2005, 287, 191-197). We speculate that the phase-separated form of liposome is more stable that the homogenously mixed lipid form, obtained immediately after sonication-extrusion. Rapid and prolonged cooling subsequent to this initial formation step seems to facilitate quicker stabilization.

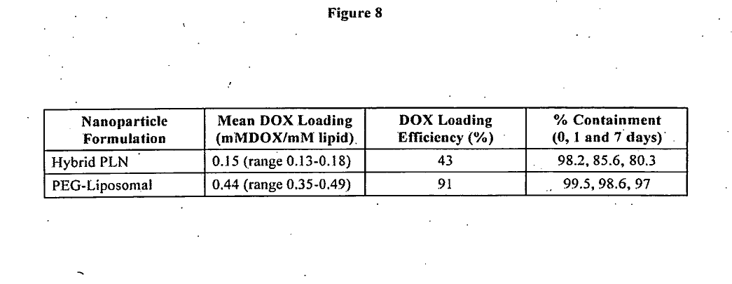

From these studies, an optimized hybrid HPLN formulation was empirically derived consisting of h-PEG₁PCDA, hydrogenated soy phosphatidyl-choline, cholesterol and m-PEG₂₀₀₀-DSPE at a molar proportion of 15:47:32:6. Using this formulation, HPLNs were fabricated and their ability to be actively loaded with doxorubicin through generation of an ion gradient, was assessed (Elijah M. Bolotin, “Rivka Cohen,” Liliana K. Bar, a Noam Emanuel, “Sami Ninio,” Danilo D. L. asic, b and Yechezkel Barenholza* AMMONIUM SULFATE GRADIENTS FOR EFFICIENT AND STABLE REMOTE LOADING OF AMPHIPATHIC WEAK BASES INTO LIPOSOMES AND LIGANDOLIPOSOMES. JOURNAL OF LIPOSOME RESEARCH, 4(1), 455-479 (1994). Using this method, doxorubicin could be loaded into HPLNs to an average final drug/lipid molar ratio of 0.15 (range 0.13-0.18) in comparison to conventional PEG-liposomes lacking PCDA lipids which could be loaded to an average molar ratio of 0.44 (range 0.35 to 0.49). Containment studies of loaded HPLNs stored at 4° C., revealed that over the course of one week, greater than 80% of the doxorubicin remained encapsulated in comparison to PEG-Liposomal formulations which had greater than 97% containment at one week (FIG. 8).

### Discussion

Though vesicles composed entirely of diacetylene containing lipids had excellent detection properties, they had limited capability as therapeutic delivery vehicles. We were unable to stably load these liposomes with doxorubicin either by passive encapsulation during vesicle formation or actively across ion gradients in formed vesicles. Others have been able to passively load hybrid liposomes composed of a 1:1 mixture of a phosphotidylcholine derivative with a di-chain diacetylene lipid and another phospholipid (Guo et al, Colloids and Surfaces B—Biointerfaces, 2010). However loading efficiencies were low and this strategy may be limited to hydrophobic payloads. We have found that for amphiphilic molecules such as doxorubicin in HPLNs, with single-chain, neutral PCDA lipids, the polymerizable lipid concentration needs to be 20 mole percent or less for efficient loading to occur (data not shown).

### Example 3

### Untargeted Doxorubicin-Loaded HPLNs are More Cytotoxic to Osteosarcoma Cells than Liposomal Doxorubicin Formulations

In this example, hybrid liposomes with recombinant anti-ALCAM antibody are shown to further improve cytotoxic killing of osteosarcoma cell lines. Since doxorubicin is a mainstay in the current treatment of osteosarcoma, it was chosen as our initial payload to test whether HPLNs could serve as therapeutic delivery vehicles. HPLNs and standard liposomes were fabricated by hydration of dried lipid films by brief sonication followed by extrusion through 100 nm polycarbonate filters as described in Example 1 above. The sizes of HPLNs and liposomes were approximately the same varying from batch to batch from 90 to 110 nm with a typical polydispersity index of about 0.1. Both particles were loaded with doxorubicin using ammonium sulfate gradients (Haran et al., Transmembrane ammonium sulfate gradients in liposomes produce efficient and stable entrapment of amphipathic weak bases. Biochim Biophys Acta, 1993). Prior to dosing cells, loaded nanoparticles were incubated briefly with an anionic exchange resin (BioRex 70, BioRad Inc) to scavenge any nonencapsulated (free) doxorubicin. Nonconfluent osteosarcoma cell lines were then incubated for 4 hours with varying concentrations of doxorubicin-loaded HPLNs or liposomes in triplicate. Cells exposed to free doxorubicin served as a positive controls. After dosing, cells were washed with fresh media and incubated for a total of 72 hours. Cell viability was then quantified by MTT assay and 50% inhibitory concentrations (IC50s) were calculated. For each osteosarcoma cell line, this experiment was performed 3-7 times using at least two different batches of HPLNs and liposomes.

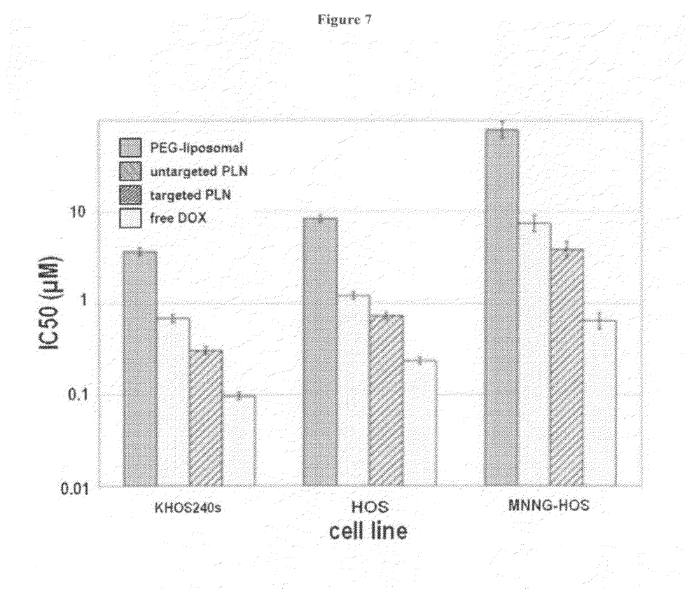

Absolute IC50 values for each doxorubicin preparation varied according to osteosarcoma cell line (FIG. 7). However the trend reflecting the relative potency of these preparations, were consistent across all cell lines tested. As has been seen previously in other cell models, free doxorubicin was approximately 50-fold more potent than liposomal doxorubicin (need reference). Loaded HPLNs (HPLN/Dox) showed intermediate potency that was about 6-fold greater than the conventional pegylated liposomal preparation.

Follow up experiments were performed to determine whether the increased growth inhibition mediated by HPLN/Dox was related to the amount of PCDA lipid in this formulation. HPLN/Dox with reduced PCDA were fabricated and incubated with the KHOS240S osteosarcoma cell line. Though these variant HPLN/Dox formulations were of similar size and loaded equally well with doxorubicin, decreasing the PCDA lipid composition resulted in nanoparticles with decreased growth inhibitory potency (data not shown).

### ALCAM Targeting Enhances the Growth Inhibitory Effect of Doxorubicin-Loaded PLNs.

We have previously shown that coupling anti-ALCAM diabodies to the surface of PLNs increases their binding affinity for osteosarcoma cell lines. This same effect was found using α-AL-HPLNs (data not shown). Experiments were then performed to determine whether this targeting function improved the ability of doxorubicin-loaded HPLNs to inhibit growth of osteosarcoma cell lines. Targetable HPLNs were fabricated using h-Peg₁-PCDA, hydrogenated soy phosphotidyl-choline, cholesterol, Mal-PEG₂₀₀₀-DSPE, and m-PEG₂₀₀₀-DSPE at a molar ratio of 15:47:32:4.5:1.5. The proportion of maleimide DSPE was empirically determined as the lowest amount that when coupled to anti-ALCAM diabody, would result in enhanced binding to osteosarcoma cells (data not shown). After loading with doxorubicin, HPLNs were coupled to anti-ALCAM diabody as before giving α-AL-HPLN/Dox. Osteosarcoma cells were then incubated with α-AL-HPLN/Dox as previously described.

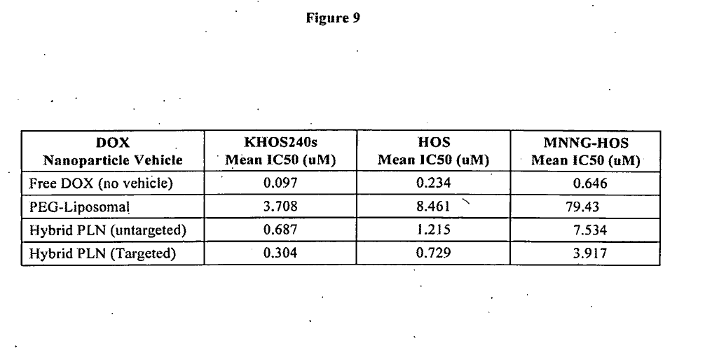

As seen with untargeted hybrids, the absolute sensitivity to α-AL-HPLN/Dox varied across different osteosarcoma cell lines (FIG. 9). However in all cases, the targeted HPLNs demonstrated an additional growth inhibitory potency over untargeted HPLN counterparts of approximately 2-fold in all cell lines (FIG. 7). Taken together, α-AL-HPLN/Dox had a log order (12-fold) increase in cytotoxicity over the conventional untargeted PEG-liposomal doxorubicin formulation in KHOS240s and HOS cell lines while having a 20-fold increase in the chemoresistant MNNG-HOS cell line. This implies that α-AL-HPLN/Dox can both specifically bind cells and deliver doxorubicin to achieve greater cytotoxicity over a conventional untargeted liposomal nanoparticle formulation.

### Discussion

Though our HPLNs were initially formulated for their stable drug loading characteristics, they surprisingly also proved to be more therapeutically potent in in vitro testing. The 1050 concentrations of untargeted doxorubicin loaded hybrid PLNs in three independent osteosarcoma tumor derived cell lines, were at least 6-fold lower that standard liposomal doxorubicin composed of PEGylated saturated phospholipid. This boost in potency appears to depend on PCDA lipid content since it is progressively lost as the PCDA concentration is titrated down from an optimum of 15-20 mole percent (data not shown). From this point, the lower the PCDA lipid concentration is in our HPLNs, the higher the 1050 becomes in our osteosarcoma model. Recently, others have used mixtures of diacetylene lipids and phospholipids to create liposomes that could be selectively destabilized either by photochemical means or by thermal shock (Yavlovich et al. J Therm Anal calorim (2009) 98(1):97-104; Yavlovih et al. Biochim Biophys Acta. (2011) 1808(1):117-26; Guo et al. (2009), Langmuir, 2009, 25 (22), 13114-13119). The goal here was to create a therapeutic vehicle that would release its payload in a temporal-spatially controlled fashion.

We have found that even without applying an external destabilizing stimulus, HPLNs can be more effective therapeutic delivery vehicles than standard liposomal formulations. The mechanisms underlying this effect are unclear and require further investigation. The presence of PCDA in our hybrid formulations could be having an effect at multiple steps in our in vitro assay from (i) nanoparticle binding to cells to (ii) cellular uptake to (iii) intracellular release of cytotoxic payload. This last step in particular may be rate limiting. The 50-fold difference in 1050 between free doxorubicin and standard liposomal doxorubicin seen in our osteosarcoma cell lines, is consistent with that found in previously published model systems. See Haglund C. et al. (1986) Br. J. Cancer 53:189-195. Others have shown that this is primarily due to delayed release of free drug from the endocytic compartment of cells that have taken up liposomal doxorubicin (de Menezes D E L, Kirchmeier M J, Gagne J F, Pilarski L M, Allen T M 1999 Cellular trafficking and cytotoxicity of anti-CD19-targeted liposomal doxorubicin in B lymphoma cells. Journal of Liposome Research 9:199-228).

It is tempting to hypothesize that the PCDA lipids may enhance the release of doxorubicin from HPLNs that have been taken up by osteosarcoma cells. Given their differences in molecular structure, it is highly likely that microsegregation occurs between PCDA lipids and phospholipid molecules on the surface of HPLNs. Phase separations in mixtures of diacetylene lipids and phospholipids have been previously demonstrated in a number of model systems (Gaboriaud et al. (2005) J Colloid Interface Sci. 1; 287(1):191-7). It is possible that these PCDA lipid islands could serve as destabilization points that could enhance drug release when exposed to intracellular environments.

The creation of an osteosarcoma targeted doxorubicin loaded HPLN (α-AL-HPLN/Dox) resulted in a 2-fold increase in cytotoxicity over the untargeted HPLN/Dox, and a 12-fold increase in cytotoxicity over the conventional PEG-liposomal formulation in the HOS and KHOS240s osteosarcoma cell lines. These results suggest that ALCAM targeting in osteosarcoma adds an incremental therapeutic effect. Interestingly, in the MNNG-HOS cell line the α-AL-HPLN/Dox had an even greater (20 fold increase) in cytotoxicity over the PEG-liposomal formulation. The MNNG-HOS cell line has high expression levels of the multi-drug resistant protein 1 (MDR1) conferring chemotherapeutic resistance to doxorubicin (Gomes C M, van Paassen H, Romeo S, Welling M M, Feitsma R I, Abrunhosa A J, Botelho M F, Hogendoorn P C, Pauwels E, Cleton-Jansen A M 2006 Multidrug resistance mediated by ABC transporters in osteosarcoma cell lines: mRNA analysis and functional radiotracer studies. Nucl Med Biol 33:831-840). The increased sensitivity of the MNNG-HOS chemoresistant cell line to the α-AL-HPLN/Dox formulation over the conventional formulation points to a therapeutic effect that may overcome multidrug resistance. We can hypothesize that the targeting and improve sustained drug release characteristics of our α-AL-HPLN/Dox formulation may help to bypass or overwhelm the drug efflux proteins mediating chemoresistance thereby improving cytotoxicity.

In conclusion, we have found a novel surface marker in human osteosarcoma, ALCAM, which we have used to specifically target osteosarcoma cells with a novel engineered drug-loaded hybrid PLN formulation anti-ALCAM immunoconjugate. These α-AL-HPLN/Dox particles show improved cytotoxicity over a conventional untargeted PEG-liposomal doxorubicin formulation and show promise as a potential therapeutic delivery platform in osteosarcoma. This new liposomal nanoparticle formulation is particularly attractive for its potential therapeutic application in resistant, refractory, and metastatic osteosarcoma where current standard systemic untargeted chemotherapy is generally not efficacious and prognosis is dismal. Furthermore, the bystander and dose-limiting side effects of systemic chemotherapy are substantial. Thus far this formulation has only been tested in tissue culture based assays, so further assessment in tumorigenic animal models is a crucial next step to validate these findings. These experiments are currently under way.

### Example 4

### Treating Melanomas Using Liposomal Nanoparticles

Liposomal nanoparticles containing mal-Peg2000-DSPE can be produced as described in the examples above. Peptides that comprise an RGD sequence or a variant thereof can be covalently linked via an extra cysteine residue to mal-Peg2000-DSPE. The RGD sequence selectively binds to integrins on the surface of tumor or angiogenic cells. The liposomal nanoparticles will comprise lipid molecules containing diacetylene residues in their acyl chain, and will be treated with UV radiation to crosslink the diacetylene residues and allow the nanoparticle to fluoresce at 635 nm. The nanoparticles will also comprise a chemotherapeutic agent, such as 5-fluorouracil or cisplatin. The nanoparticles can be tested for targeting to NW-145 cells, a human melanoma cell line using the methods described in the examples above. Nanoparticles will be added at varying concentrations to NW-145 cells and to control cells. Unbound nanoparticles will then be washed off and bound nanoparticles detected by fluorescence microscopy. The cells can also be incubated over several hours or days, and cytotoxicity of the nanoparticles measured by observing cell death.

### Example 5

### Clinical Trial Using Liposomal Nanoparticles to Treat Osteosarcoma

Liposomal nanoparticles of the invention can be produced to comprise two tumor-associated markers, ALCAM and hyaluronan receptor (CD166 and CD44, respectively) to enhance targeting specificity as described in the examples above. Selected osteosarcoma patients will be randomly divided into two groups. One group will be treated with a standard combination of doxorubicin, cisplatin, and methotrexate (together called MAP). The other group will be divided into cohorts of 6 people and treated with increasing doses of targeted liposomal nanoparticles comprising all three MAP components: Treatment will comprise courses of targeted liposomal nanoparticles over 30-60 minutes on day 1, with courses repeated at increasing concentrations every 21-28 days; to a maximum cumulative dose of 500 mg/m² per doxorubicin. Side effects from both the nanoparticle and the standard treatments will be measured, and the maximum tolerated dose (MTD) of liposomal nanoparticles determined. MTD will be defined as the dose preceding that at which 1 of 6 patients experience dose-limiting toxicity. Standard measurements will also be used to follow disease progression.

This clinical trial can be followed by a phase II trial, wherein additional patient cohorts are recruited and treated with the liposomal nanoparticles at the determined MTD. All patients will be followed up for at least 2 years post-study. If treatment is successful, additional follow-up studies can be performed to determine whether patients treated with the liposomal nanoparticles have a reduced chance of developing secondary malignancies compared to patients treated with conventional MAP chemotherapy.

### Example 6

### Using Liposomal Nanoparticles for PET Imaging of Pancreas Cancer

In this example, an anti-CA19-9 cys-diabody fragment was engineered to target nanoparticles to pancreatic cancer.

Antibodies are a unique class of targeting agents capable of exquisite specificity for cell surface antigens. The smallest engineered antibody fragment that retains antigen specificity is the single-chain Fv (scFv) fragment consisting of a variable light (V_{L}) chain and variable heavy (V_{H}) chain joined by an amino acid linker (Kenanova et al., Tailoring antibodies for radionuclide delivery. Expert Opin Drug Deliv, 2006). The length of the linker sequence can be adjusted to promote the formation of the diabody, a noncovalent dimer of 2 scFv chain (Kortt et al., Dimeric and trimeric antibodies: high avidity scFvs for cancer targeting. Biomol Eng, 2001). The diabody has been shown to exhibit superior binding affinity and avidity compared to the scFv, likely because of its bivalency for its antigen (Adams et al., Avidity-mediated enhancement of in vivo tumor targeting by single-chain Fv dimers. Clin Cancer Res, 2006). Moreover, a covalent dimer of 2 scFv chains (cys-diabody) can be created by engineering C-terminal cysteine residues into the DNA construct of the scFv (Olafsen et al., Covalent disulfide-linked anti-CEA diabody allows site-specific conjugation and radiolabeling for tumor targeting applications. Protein Eng Des Sel, 2004). The cys-diabody has the ability to be site-specifically conjugated with fluorophores or radioisotopes through the free sulfhydryl groups of the cysteine residues after reduction of the disulfide bond (Olafsen et al., 2004). Work by Carmichael, et al elucidated the crystal structure of the anti-CEA diabody and showed that the C-termini are at the opposite end of the protein's antigen binding pocket making it an appealing site to engineer the cysteine residues (Carmichael et al., The crystal structure of an anti-CEA scFv diabody assembled from T84.66 scFvs in V(L)-to-V(H) orientation: implications for diabody flexibility. J Mol Biol, 2003). This study further demonstrated that modification in this manner did not alter the diabody's affinity once labeled to a radionuclide through those cysteine residues (Carmichael et al., 2003). The unique ability to conjugate site-specifically to these cysteine residues makes the cys-diabody fragment an attractive fragment for therapeutic applications. In particular, it can be used for site-specific conjugation of therapeutic nanoparticles and thus have the potential to deliver targeted therapy to cancer cells.

The goal of this example was to modify an anti-CA19-9 diabody by engineering cysteine residues into the protein, to characterize the newly generated cysteine modified anti-CA19-9 diabody (anti-CA19-9 cys-diabody) and to confirm its ability to retain its targeting potential by itself as well as while conjugated to PLNs. To do this, we first engineered the cysteine residues into the C-terminus of the anti-CA19-9 diabody and evaluated its antigen specific binding ability in vitro and in vivo utilizing microPET/CT. Next, we conjugated the anti-CA19-9 cys-diabody to the surface of the nanoparticle via the free sulfhydryl groups of the reduced cysteine residues. Finally, we evaluated the ability of the anti-CA19-9 cys-diabody-PLN conjugate to discriminate CA19-9 positive cells from negative cells using immunofluorescence studies and flow cytometry.

### Methods

### Materials

Polymerized Liposomal Nanoparticles (PLNs) were obtained from NanoValent Pharmaceuticals, Inc. (Bozeman, Mont.). The lipids comprising the PLNs are N-(5′-hydroxy-3′-oxypentyl)-10-12-pentacosadiynamide (“h-Peg₁-PCDA”), N-[methoxy(polyethylene glycol)-750]-10-12-pentacosadiynamide (“m-Peg₇₅₀-PCDA”), N-(5′-sulfo-3′-oxypentyl)-10,12-pentacosadiynamide, sodium salt (“sulfo-Peg₁-PCDA”) and N-[maleimide(polyethylene glycol)-1500]-10-12-pentacosadiynamide (“mal-Peg₁₅®-PCDA”).

Construction of the Anti-CA19-9 Cys-Diabody

The scFv DNA fragment in the pUC18 vector (New England Biolabs, Beverly, Mass.) engineered previously for the anti-CA19-9 diabody was used as the template to perform PCR and add the cysteine residues into the C-terminal end of the scFv (Sirk et al., Site-specific, thio-mediated conjugation of fluorescent probes to cysteine-modified diabodies targeting CD20 or HER2. Bioconjug Chem, 2008). This was accomplished using standard PCR reaction components (Invitrogen, Carlsbad, Calif.) with a 48 base pair primer to insert the GGCCG amino acid sequence (5′-GAATTCTCAATGATGATGATGATGATGACCCCCACACCCACCTGCAGA-3′, IDT Integrated DNA Technologies, San Diego, Calif.). This construct was confirmed by DNA sequencing, excised from the pUC18 vector, and ligated into the pEE12 mammalian expression vector (Lonza Biologics, Slough, United Kingdom) containing the glutamine synthetase gene for selection and the hCMV promoter for high, expression.

### Expression, Selection, and Purification

NS0 murine myeloma cells (5.0×106) were transfected by electroporation with 20 μg of linearized pEE12 scFv DNA construct. Cells were selected in glutamine deficient DMEM/high modified media (JRH Biosciences, Lenexa, Kans.) as previously described (Galfre and Milstein, Preparation of monoclonal antibodies: strategies and procedures. Methods Enzymol, 1981; Kenanova et al., Tailoring the pharmacokinetics and positron emission tomography imaging properties of anti-carcinoembryonic antigen single-chain Fv-Fc antibody fragments. Cancer Res, 2005). Transfectants were screened for expression of cys-diabody by Western blot in which the nitrocellulose membrane (Bio-Rad Laboratories, Hercules, Calif.) was incubated sequentially with 1 μg of anti-Penta His IgG antibody (Sigma-Aldrich, Bellefonte, Pa.) and 1 μg alkaline-phosphatase conjugated goat anti-mouse IgG, Fc specific, antibody (Jackson ImmunoResearch Labs, West Grove, Pa.) and developed with BCIP/NCP Color Development Substrate (Promega, Madison, Wis.). Based on Western blot analysis, high producing clones were selected for expansion into triple flasks (Nunclon, Rochester, N.Y.). After cell growth and exhaustion in the triple flask stage, supernatant was harvested and centrifuged to remove cellular debris then applied onto a 1 ml HiTrap Chelating HP column (GE Healthcare, Piscataway, N.J.) at a flow rate of 1 ml/min using an AKTA Purifier (GE Healthcare) for cys-diabody purification. Bound proteins were eluted with 250 nM imidazole in PBS. Fractions containing the cys-diabody were evaluated with SDS-PAGE, pooled, and dialyzed against PBS using a Slide-A-Lyzer Dialysis Cassette (Thermo Fisher Scientific, Rockford, Ill.) with a molecular weight cutoff of 10,000 daltons to buffer exchange purified proteins and ensure no impurities. The purified and buffer exchanged cys-diabody was then concentrated using a spin column (Vivaspin 20, 10 kDa cutoff, Thermo Fisher Scientific), and the final cys-diabody protein was stored at 4° C.

### Biochemical Characterization of Purified Anti-CA19-9 Cys-Diabody Protein

The purified anti-CA19-9 cys-diabody was analyzed with SDS-PAGE on pre-cast 4-20% gels (Bio-Rad Laboratories). Protein samples were incubated with and without the reducing agent, dithiothreitol (DTT), at a concentration of 100 mM of DTT to ensure efficient reduction of the disulfide bond. Gels were stained with Microwave Blue™ (Protiga Inc., Frederick, Md.) for detection of proteins. Samples were also analyzed with size exclusion chromatography (SEC) on a Superdex 75 HR 10/30 column (GE Healthcare). Approximately 50 μg of protein was applied to the column and run isocratically in PBS at a flow rate of 0.5 ml/min on the AKTA Purifier. Elution time was obtained and compared to carbonic anhydrase (30 kDa) and bovine serum albumin (BSA, 66 kDa) standards (Sigma).

### Cell Lines

NS0 mouse myeloma cells were maintained with DMEM/high modified media supplemented with 10% fetal bovine serum (FBS, Invitrogen) and 1% glutamine (Invitrogen). The human pancreatic cancer cell lines, BxPC3 and MiaPaca-2 (American Type Culture Collection, ATCC, Manassas, Va.) were maintained in RPMI-1640 and DMEM, respectively. All media was supplemented with 1% penicillin/streptomycin (Invitrogen) and 10% FBS. Media for the MiaPaca-2 cell line was also supplement with 2.5% horse serum (Invitrogen) as recommend by the ATCC.

### Preparation of Polymerized Liposomal Nanoparticles (PLNs)

Liposomes were prepared from h-Peg1-PCDA: m-Peg750-PCDA: sulfo-Peg1-PCDA: mal-Peg1500-PCDA (6.7:2.6:0.5:0.2, molar ratio) according to the method previously described (Bruehl, et al., Polymerized liposome assemblies: bifunctional macromolecular selectin inhibitors mimicking physiological selectin ligands. Biochemistry, 2001). Briefly, lipids were mixed and evaporated in vacuo, to a film. Deionized water was added to the films so as to give a 24 mM (total lipid) suspension. The suspension was heated via sonication between 70-80° C. with a probe-tip sonicator (Fisher sonic dismembrator model 300) for 10 min. The resulting clear solution was then cooled to 5° C. for 20 min., warmed to ambient temperature for 20 min. and polymerized by UV light irradiation (254 nm) with a Spectrolinker XL-1000 UV Crosslinker (Spectronics Corp.) for 10 minutes. The resulting blue PLNs were heated to 65° C. for 5 min to convert them to the red (fluorescent) form. The colored solutions were syringe filtered through 0.2 μm cellulose acetate filters in order to remove trace insoluble contaminants. The average particle size measurements were obtained on a Zetasizer Nano-S (Malvern Inst.), in a solution of 10 mM sodium chloride.

### Conjugation of the Anti-CA19-9 Cys-Diabody to PLNs

The anti-CA19-9 cys-diabody was conjugated to PLNs using maleimide chemistry. Approximately 50 μg of cys-diabody in 50 μl of PBS was reduced with 100 μl of immobilized tris(2-carboxyethyl)phosphine (TCEP) for 30 minutes at room temperature. The TCEP was subsequently spun out of solution in a cellulose spin cup (Thermo Fisher Scientific). The reduced cys-diabody was added immediately to a solution of PLNs (NanoValent Pharmaceuticals, Inc., Bozeman, Mont.) at a ratio of 50 μg of cys-diabody to 250 μg of PLNs This mixture was incubated at room temperature for 2 hours and dialyzed overnight in PBS buffer using Slide-A-Lyzer Dialysis Cassette (MW cutoff, 100 kDa) to remove any free unbound cys-diabody.

### In Vitro Binding Assays by Flow Cytometry and Immunofluorescence

Flow cytometry and immunofluorescence was used to assess the binding ability of both the anti-CA19-9 cys-diabody and the anti-CA19-9 cys-diabody-PLN conjugate. All human pancreatic cell lines were harvested (1×10⁶ cells), resuspended in 250 μl of PBS/2% FBS and incubated with 4 μg of cys-diabody on ice for 1 hour. The cells were washed for 10 minutes by centrifugation at 1000×g, the supernatant was discarded, and the cells were resuspended in another 250 μl of PBS/2% FBS. The cells were again incubated with secondary antibody, 4 μg of anti-Penta His IgG antibody (Sigma) for 1 hour on ice. Another wash was performed and the cells were finally incubated for 1 hour on ice with 4 μg of tertiary antibody, R-phycoerythrin (R-PE) conjugated goat anti-mouse IgG, Fc specific, antibody (Jackson Immunoresearch Labs). A final wash was performed and the cells were resuspended with 500 μl PBS/2% FBS. Binding data was obtained using a ScanX flow cytometer (Becton Dickinson, Franklin Lakes, N.J.) and analyzed using FlowJo software (Tree Star Inc., Ashland, Oreg.).

Similarly, immunofluorescence was performed using 6-well plates (Becton Dickinson) with sequential incubation steps of the anti-CA19-9 cys diabody, secondary antibody, and tertiary antibody with wash steps in between incubations. Once completion of all steps, the wells were visualized with a Nikon 90S fluorescent microscope (Nikon Inc., Melville, N.Y.) and images were taken with a 5.1 megapixel CCD camera (Nikon). Images were analyzed using Nikon software and were processed for contrast and brightness using Photoshop Elements 4 (Adobe Systems Inc., San Jose, Calif.).

For both flow cytometry and immunofluorescence, negative control samples included samples with cells only or cells incubated with secondary and tertiary antibody and no primary antibody. Experimental samples were cells incubated with cys-diabody, secondary, and tertiary antibodies. The positive control samples were those with cells incubated with the parental intact mAb and the tertiary antibody.

Additionally, flow cytometry and immunofluorescence were performed in the same manner using the anti-CA19-9 cys-diabody-PLN conjugate as the primary antibody. Because the PLN exhibits autofluorescence in the same ultraviolet range as R-PE, no secondary or tertiary antibodies were used. Experiments included cells only, cell incubated with cys-diabody-PLN conjugate, and cells incubated with PLNs only as a negative control.

### Cell-Based Competition ELISA

To determine the relative binding affinity of the anti-CA19-9 cys-diabody, a cell-based competition ELISA was performed. CA19-9 positive cells, BxPC3, were harvested, counted, and aliquoted into a 96-well plate (50,000 cells/well) for adhesion overnight. The following day, the cell media was removed and each well was washed with 150 μl of PBS/2% FBS. The wash was also removed and each well was incubated for 1 hour at room temperature with a 150 μl of a 1 nM concentration of the parental intact anti-CA19-9 mAb obtained from purifying the supernatant of the 116-N-19-9 hybridoma cells known to produce the mAb (Koprowski et al., Colorectal carcinoma antigens detected by hybridoma antibodies. Somatic Cell Genet, 1979). After incubation, the wells were washed similarly and incubated for 1 hour at room temperature with 150 μl of varying concentrations, ranging 0.01 nM to 100 nM, of the anti-CA19-9 cys-diabody. Again, the wells were washed and incubated with 150 μl of a 1:2500 μl dilution of AP-conjugated goat anti-mouse IgG, Fc specific, antibody (Jackson Immunoresearch Labs). After 1 hour, the wells were washed again and developed with 150 μl of a solution of 10 mg phosphatase tabs (Sigma) dissolved in 10 ml AP buffer. The reaction was permitted to proceed for 15 minutes and then evaluated for ultraviolet absorbance using the GENios microplate reader (Tecan, Durham, N.C.). All experiments were done in triplicate. A saturation binding plot was created based on the ultraviolet absorbance value of each sample in order to calculate the dissociation constant (Kd), defined as the amount of cys-diabody needed to displace 50% of the parental intact anti-CA19-9 mAb.

### Radioiodination

Radiolabeling of the anti-CA19-9 cys-diabody with the positron-emitting isotope, iodine-124 (¹²⁴I), was performed using the Iodo-Gen method as described (Richardson et al., An improved iodogen method of labelling antibodies with ¹²³I. Nucl Med Commun, 1986). The labeling reaction was performed with 200 μg of purified cys-diabody (200 μl) and 800 μgCi of Na¹²⁴I (IBA Molecular, Dulles, Va.). Instant thin layer chromatography (TLC) using the Tec-Control kit (Biodex Medical Systems, Shirley, N.Y.) was used to measure labeling efficiency. Immunoreactivity, defined as the fraction of cys-diabody retaining the ability to bind to cells after radiolabeling, was determined by incubating the radioiodinated cys-diabody (100,000 cpm) with BxPC3 cells and MiaPaca-2 cells in PBS/2% FBS such that there was an excess of antigen for the positive cell line. MiaPaca-2 cells, negative for CA19-9, were used to confirm no change in binding of cys-diabody after radiolabeling. The radioiodinated cys-diabody was allowed to incubate for 1 hour at room temperature and washed with PBS/2% FBS for 20 minutes. Any cys-diabody, and thus radioactivity, not bound to cells was collected in the supernatant and measured in a Wizard 3′ 1480 Automatic Gamma Counter (Perkin-Elmer, Covina, Calif.). This fraction was divided by the total radioactivity incubated with cells, yielding the portion of unbound radioactivity (i.e. unbound cys-diabody). This fraction was subtracted from 1 to equal the portion of bound cys-diabody (i.e. Immunoreactivity).

### Xenograft Imaging and Biodistribution Studies

Animal handling was carried out under a protocol approved by the Chancellor's Animal Research Committee of the University of California in Los Angeles (UCLA). The antigen positive (BxPC3) xenograft models were established with 1×10⁶ cells injected subcutaneously into the left shoulder of 4 mice. In the same 4 mice, the antigen negative cell line (MiaPaca-2) was injected in the right shoulder as a negative control. Tumors were allowed to develop for approximately 3 weeks. Gastric lavage was performed with 1.5 mg of potassium perchlorate in 200 μl of PBS 30 minutes prior to tail vein injection of ¹²⁴I-anti-CA19-9 cys-diabody. Additionally, blocking of thyroid uptake of radioiodine was accomplished by adding saturated potassium iodide (0.5 ml per 100 ml water) to the drinking water 24 hours prior to injection of the radioiodinated cys-diabody. Mice were injected with approximately 25 μg of ¹²⁴I-anti-CA19.9 cys-diabody (specific activity of 4.03±1.8 μCi/μg) in PBS/2% FBS via the tail vein. MicroPET was performed at 4 and 20 hours post-injection. These time points for imaging were chosen based on previously measured half-lives of diabody fragments and corresponded to approximately 1 half-life and 5 half-lives, respectively (Olafsen et al, 2004; McCartney et al., Engineering disulfide-linked single-chain Fv dimers [(sFv′)2] with improved solution and targeting properties: anti-digoxin 26-10 (sFv′)2 and anti-c-erbB-2 741F8 (sFv′)2 made by protein folding and bonded through C-terminal cysteinyl peptides. Protein Eng, 1995; Williams et al., Numerical selection of optimal tumor imaging agents with application to engineered antibodies. Cancer Biother Radiopharm, 2001). Also, to appropriately compare the in vivo targeting ability of the anti-CA19-9 cys-diabody to the anti-CA19-9 diabody, we performed the imaging experiments at the same time points as previously published for the anti-CA19-9 diabody (Girgis et al., Anti-CA19-9 diabody as a PET imaging probe for pancreas cancer. J Surg Res, 2011 170:169-178, which is herein incorporated by reference in its entirety). The mice were anesthetized using 2% isoflurane, placed on the microPET bed, and imaged with a Focus microPET scanner (Concorde Microsystems Inc., Knoxville, Tenn.). Acquisition time was 10 min. All images were reconstructed using a FBP algorithm and displayed by the AMIDE software package (Defrise et al., Exact and approximate rebinning algorithms for 3-D PET data. IEEE Trans Med Imaging, 1997; Loening and Gambhir, AMIDE: a free software tool for multimodality medical image analysis. Mol Imaging, 2003). One animal was also imaged by micro computed tomography (microCT) and coregistered with microPET images for anatomic reference. Animals were euthanized after the last imaging time point and organs, tumors and blood were harvested and weighed. Radioactive uptake of organs was counted in a gamma counter for biodistribution analysis and converted to a percentage of injected dose of radioactivity per gram (% ID/g) of tissue after decay correction.

### Results

### Construction of the Anti-CA19-9 Cys-Diabody

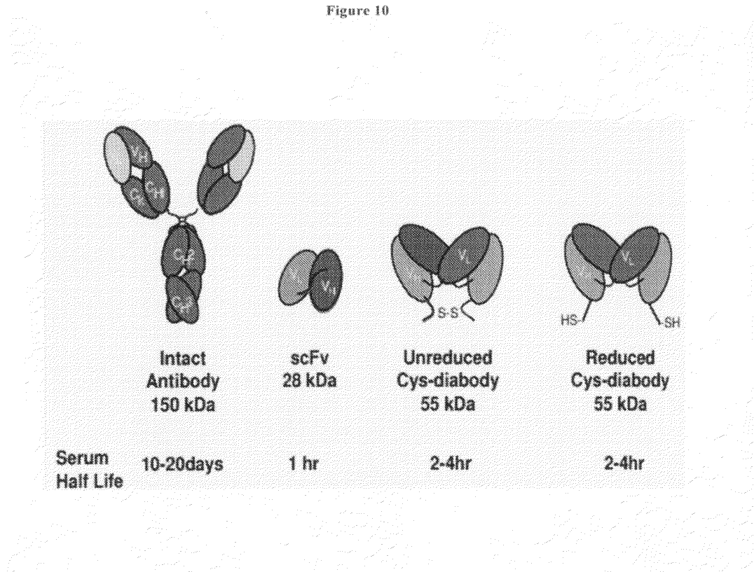

Using the anti-CA19-9 diabody DNA as a template, PCR was performed to insert the C-terminal cysteine residues. Gene sequencing and comparison to the published sequence confirmed success (Tonge et al., Cloning and characterization of 1116NS19.9 heavy and light chain cDNAs and expression of antibody fragments in Escherichia coli. Year Immunol, 1993). The resultant DNA construct used for transfection of NS0 cells is shown in FIG. 10B.

### Expression, Selection, and Purification

The cys-diabody was expressed in NS0 myeloma cells. Cell culture supernatant was harvested and analyzed with Western blot to determine those clones expressing the highest levels of protein. These were selected for expansion into triple flasks (Nunclon). Selected clone expression ranged from 10 to 45 mg/L. The 6×His tag engineered on the C-terminus of the anti-CA19-9 cys-diabody was used for protein purification. Approximately 100 ml of culture supernatant yielded 1-4.5 mg of pure protein.

### Biochemical Characterization of the Anti-CA19-9 Cys-Diabody.

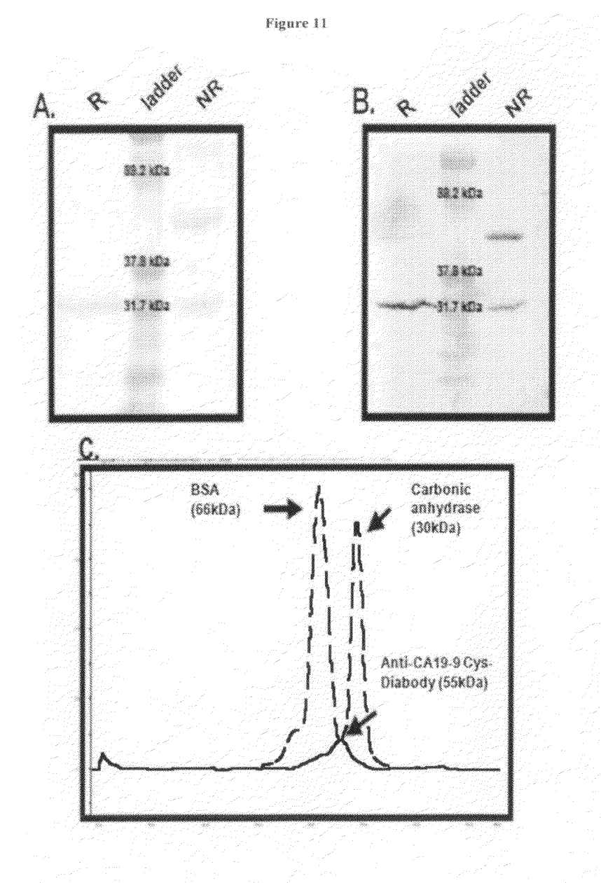

Purity of the isolated cys-diabody obtained from high performance liquid chromatography (HPLC) purification (FIGS. 11A and 11B) was confirmed by SDS-PAGE and Western blot. SEC was done to evaluate for the presence of the appropriate covalent interactions between 2 scFv fragments to form the cys-diabody (FIG. 11C). As shown, compared to BSA (20.75 minutes) and carbonic anhydrase (24.75 minutes) standards, the cys-diabody elution time was 23.98 minutes. The elution time of the cys-diabody was consistent with its predicted molecular weight of 56 kDa.

### In Vitro Binding Assays for the Characterization of the Anti-CA19-9 Cys-Diabody

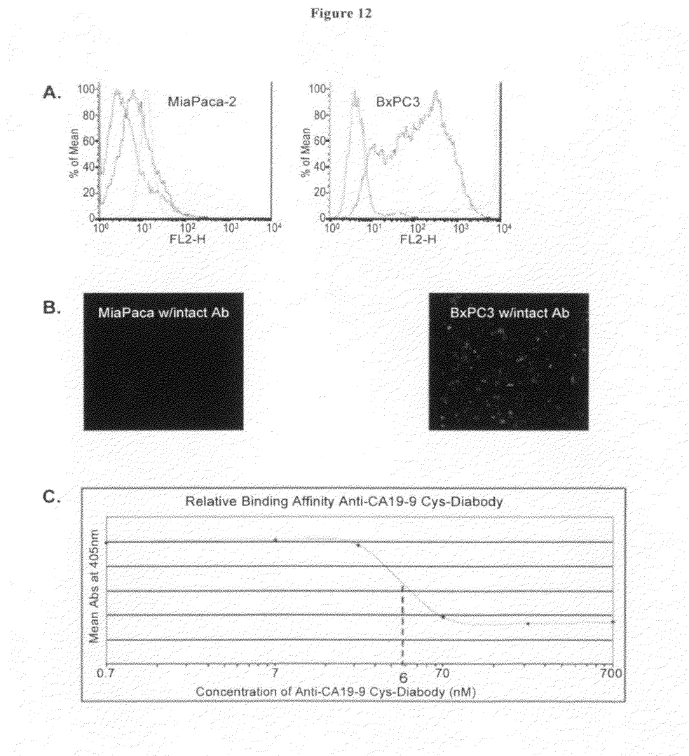

CA19-9 recognition and binding were evaluated by flow cytometry and immunofluorescence. The anti-CA19-9 cys-diabody demonstrated the ability to distinguish between the CA19-9 positive cell line, BxPC3, and the CA19-9 negative cell line, MiaPaca-2 by flow cytometry (FIG. 12A). In comparison to the parental intact anti-CA19-9 antibody, the cys-diabody exhibited slightly lower binding efficiency. MiaPaca-2 showed no expression of CA19-9 and served as the negative control. Immunofluorescence data were in accordance with the flow cytometry data (FIG. 12B). The MiaPaca-2 cell line showed no binding of the intact anti-CA19-9 mAb or cys-diabody, while the BxPC3 cell line showed comparable binding between the 2 antibodies.

### Cell-Based Competition ELISA

A competition binding assay as described was performed to ascertain the relative binding affinity of the anti-CA19-9 cys-diabody when compared to the mouse anti-CA19-9 intact antibody. The absorbance values obtained for each sample were averaged and plotted on a saturated binding plot. The observed relative binding affinity of the diabody was less than 10 nmol/L (FIG. 12C).

### Radioiodination, Xenograft Imaging, and Biodistribution Studies

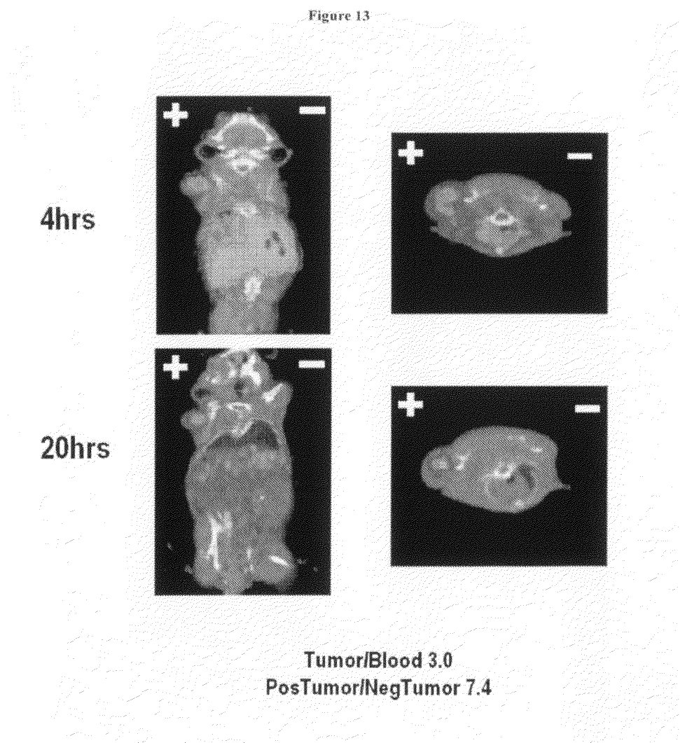

In vivo tumor targeting of the anti-CA19-9 cys-diabody was evaluated with MicroPET imaging. Nude mice carrying CA19-9 positive (BxPC3) and CA19-9 negative (MiaPaca-2) tumors were injected with the ¹²⁴I-labeled anti-CA19-9 cys-diabody. The labeling efficiency of ¹²⁴I on the anti-CA19-9 cys-diabody was 96%. Immunoreactivity of the ¹²⁴I-labeled anti-CA19-9 cys-diabody on BxPC3 cells was 75% and 1% on MiaPaca-2 cells. MicroPET imaging studies were conducted in 4 animals bearing BxPC3 tumors averaging 55 mg (range, 21-128 mg), obtained at 4 and 20 hours after injection of the cys-diabody. FIG. 13 illustrates representative images from each study. After the 20 hours time point, all 4 mice were sacrificed and organs were harvested for measurement of radioactivity to provide quantitative evidence of tumor targeting. These data were used to calculate tumor-to-blood ratios in each animal. For the 4 mice bearing BxPC3 tumors, the positive tumor activity averaged 0.8 percent injected dose per gram (% ID/g) with a range of 0.5-1.2% ID/g. The average negative tumor activity in this model was 0.1% ID/g (range, 0.03-0.2% ID/g). The average tumor to blood ratio was 3:1, and the positive tumor to negative tumor ratio was 8:1. High uptake was noted in the liver (data not shown).

### Construction of Maleimide PLNs and Characterization

PLNs were synthesized by the self-assembly of lipids into small, unilamellar liposomes. The polymerizable lipids contain diacetylene groups in the fatty-acid tails. Methoxy- and maleimide-terminated polyethylene glycol (PEG) polymers are appended to the liposome-forming lipids by chemically conjugating them to the carboxyl groups in the commercially available PCDA lipid (GFS Chemicals). Aqueous dispersions of the lipids are sonicated while being heated to a temperature above the lipid phase transition giving liposomes that appear as clear solutions. In order to obtain polymerized (polydiacetylene) PLNs the lipid chains must be in a tightly packed, solid analogous state as part of the membrane bilayer (Haglund et al., Gastrointestinal cancer-associated antigen CA 19-9 in histological specimens of pancreatic tumours and pancreatitis. Br J Cancer, 1986). This is achieved by cooling the liposome solution to 5° C. for 20 min. After warming, the solutions are irradiated with UV light to initiate a radical polymerization process, resulting in a deeply blue-purple colored, PLN solution.

The blue-purple form of the PLN is only slightly fluorescent and to obtain highly fluorescent particles the solutions are briefly heated. This results in a slight perturbation of the polydiacetylene polymer backbone that results in a chromatic shift in color and fluorescence (Loy et al., Distribution of CA 19-9 in adenocarcinomas and transitional cell carcinomas. An immunohistochemical study of 527 cases. Am J Clin Pathol, 1993). The fluorescence emission spectrum is centered at 635 nm with a broad and complex excitation spectrum from 480 to 580 nm. We have seen some evidence that cellular uptake of the non-fluorescing PLNs rapidly converts them into fluorescent form (unpublished data). For the cell uptake studies, however, we preheated the PLNs to convert them all into their fluorescent form. A distinct advantage to the PLN fluorescence is that little or no photobleaching occurs.

The PLNs resulting from simple probe-tip sonication of this lipid formulation give in a fairly tight population of small sized particles. Particle size analysis shows them to be typically in a size range from 30-50 nm with the average size centering around 38 nm. No attempts were made to produce tighter size ranges by membrane extrusion, although this technique is applicable to the liposome preparation step and will be conducted for material that will subsequently be administered in vivo, in future studies.

### Conjugation of the Anti-CA19-9 Cys-Diabody to PLNs

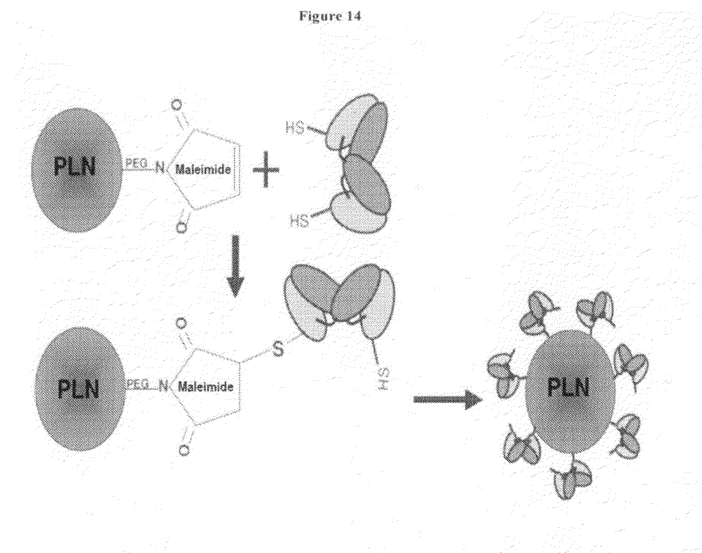

Approximately 200 of anti-CA19-9 cys-diabody was used for conjugation to 1000 μg of PLNs (FIG. 14). The reaction time was 2 hours. Reaction efficiency was approximately 80% measured by calculation of the amount of cys-diabody remaining after buffer exchange. Additionally this conjugation was stable for over 1 month's time as all experiments were duplicated 1 month after conjugation with similar results.

### In Vitro Binding Assays for Characterization of the Anti-CA19-9 Cys-Diabody-PLN Conjugate

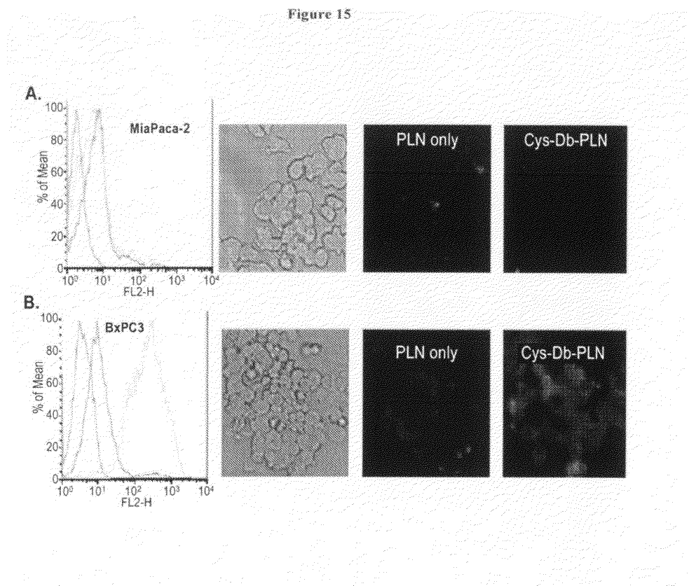

Flow cytometry of the anti-CA19-9 cys-diabody-PLN conjugate displayed similar results when compared to the unconjugated anti-CA19-9 cys-diabody (FIG. 15). The cys-diabody-PLN conjugate was able to discriminate the positive and negative cell lines by both immunofluorescence and flow cytometry. Furthermore, immunofluorescence of the cys-diabody-PLN conjugate demonstrated a pattern of localization to the outer surface of the cell membrane (FIG. 15).

### Conclusion

CA19-9 was chosen because of its prevalence on over 90% of all pancreatic cancers and its accessibility on the outer surface of cell membranes (Haglund et al., 1986; Loy et al., 1993; Makovitzky, The distribution and localization of the monoclonal antibody-defined antigen 19-9 (CA19-9) in chronic pancreatitis and pancreatic carcinoma. An immunohistochemical study. Virchows Arch B Cell Pathol Incl Mol Pathol, 1986; Ohshio et al., Immunohistochemical studies on the localization of cancer associated antigens DU-PAN-2 and CA19-9 in carcinomas of the digestive tract. J Gastroenterol Hepatol, 1990). In addition to its wide spread prevalence and accessibility, it is very abundant with expression estimated to be between 1-2 million antigens per cell. Based on these attributes, it represents an ideal tumor target. With the anti-CA19-9 diabody, we were able to demonstrate targeting of CA19-9 in vitro and in vivo (Girgis et al., CA19-9 as a Potential Target for Radiolabeled Antibody-based Positron Emission Tomography of Pancreas Cancer, Internat'l J. of Molec. Imaging, 2011, 1-9, which is herein incorporated by reference in its entirety). These results encouraged us to modify the diabody to a cys-diabody and exploit the unique ability to site-specifically conjugate to this fragment. To this end, we engineered, produced, and characterized the anti-CA19-9 cys-diabody. We show that our cys-diabody exhibits similar biochemical properties to other engineered cys-diabodies, displaying a molecular weight of 56 kDa and forming a covalent dimer (Olafsen et al., 2004; Sirk et al., Site-specific, thiol-mediated conjugation of fluorescent probes to cysteine-modified diabodies targeting CD20 or HER2. Bioconjug Chem, 2008). In addition, we demonstrate specific targeting of CA19-9 with the cys-diabody by flow cytometry and immunofluorescence with similar binding affinity compared to the parental anti-CA19-9 diabody.

To ensure that the modification of c-terminal cysteine residues did not alter the in vivo binding properties of the diabody, we investigated the ability of the cys-diabody to target CA19-9 by injecting mice harboring the BxPC3 xenograft and the MiaPaca-2 xenograft via the tail vein with ¹²⁴I labeled anti-CA19-9 cys-diabody. MicroPET images at 4 hours demonstrated quick targeting of the antibody fragment to the BxPC3 xenograft (CA19-9 positive), similar to the parental anti-CA19-9 diabody. Additionally, we showed that the cys-diabody was retained at the site of the xenograft based on the persistence of signal on microPET images at the 20-hour time point. Accordingly, biodistribution data at 20 hours after injection provides objective confirmation of the microPET images. Biodistribution data, corrected for time of injection, provided raw numerical values expressed in percent of the injected dose of radioactivity per gram of tissue (% ID/g) and was ascertained for the positive and negative tumors as well as blood and organs. As expected, the liver demonstrated a high % ID/g accounting for the presence of signal in the liver on microPET images. This phenomenon is explained best by Ahlgren, et al who characterized a Her2 affibody with and without a 6×His tag noting that the affibody with the 6×His tag had significantly increased amount of liver uptake compared to the Her2 affibody without the tag (Ahlgren et al., Targeting of HER2-expressing tumors with a site-specifically 99mTc-labeled recombinant affibody molecule, ZHER2:2395, with C-terminally engineered cysteine. J Nucl Med, 2009). Although this phenomenon has not been specifically described in humans, it represents a potential obstacle to clinical translation of proteins engineered with 6×His tags and can be overcome with alternate purification techniques not requiring the 6×His tag. More importantly, a tumor to blood ratio of 3.0 and positive to negative tumor ratio of 8.0 was obtained. These values indicate that the images display enough contrast between the tumor and blood (i.e. background) to be visualized. Not only do these data indicate antigen specific targeting of the BxPC3 xenograft but also provide evidence of the potential of the anti-CA19-9 cys-diabody as a targeting agent for pancreatic cancer.

After confirming the cys-diabody's ability to antigen specifically target CA19-9 positive cells both in vitro and in vivo, we set out to develop a site-specific conjugation reaction to add our anti-CA19-9 cys-diabody to the surface of our polymerized liposomal nanoparticles (PLNs). By reducing the disulfide bonds of the c-terminal cysteine residues, free sulfhydryls are available for conjugation to PLNs using maleimide chemistry. The maleimide group was incorporated onto the PLN surface right from the assembly stage by attaching it to one of the PLN-forming lipids. To confirm that the cys-diabody-PLN conjugate was created and that it bound to cells in an antigen specific manner, we conducted a number of studies. Given the PLN's inherent autofluorescence, we assumed that a cell with PLN bound would be captured by the flow cytometry detector without any additional method of detection. The negative cell line showed no fluorescence with the PLN alone or with the conjugate solution, and the BxPC3 cells demonstrated fluorescence only with the cys-diabody-PLN conjugate and not with the PLN alone. These results confirm that the conjugation reaction was successful and that we are able to target PLNs to cancer cells in vitro through the specific antibody-antigen interaction of the anti-CA19-9 cys-diabody. To our knowledge, we are the first to show that PLNs can be targeted to cancer cells through the interaction of a diabody and its antigen.

This achievement further defines the potential to deliver targeted treatment to cancer cells. By virtue of the ability of PLNs to serve as vehicles for chemotherapeutic agents, this could provide a method by which our targeting agent, the anti CA19-9 cys-diabody, can be applied for delivery of targeted therapy. Although further studies evaluating the ability of the cys-diabody-PLN conjugate to target pancreas cancer in vivo need to be performed, the potential for an antigen-specific technique for delivery of chemotherapeutics is promising.

In summary, diabodies are small bivalent antibody fragments are highly specific agents that can be used to target tumor antigens. In this study, we expand upon our previous results with the anti-CA19-9 diabody by engineering the anti-CA19-9 cys-diabody in order to exploit its ability to be site-specifically conjugated to PLNs. After modifying the diabody to the cys-diabody, we show that the antibody fragment retains its antigen specificity in vitro and in vivo providing antigen-specific microPET imaging of pancreatic cancer xenografts in a mouse model. Additionally, we demonstrated that the cys-diabody can be covalently conjugated to PLNs while retaining its immunoreactivity against the tumor antigen, CA19-9. This is the first report using site specific conjugation of a cys-diabody to nanoparticles opening the door to a wide variety of possible therapeutic and diagnostic applications due to the flexible nature of these liposomal vehicles. Passive targeting via the EPR effect may not be sufficient to create a significant treatment advantage at the tumor compared to normal tissues. By creating an anti-CA19-9-PLN immunoconjugate, we can achieve “active targeting” through antibody-antigen recognition, which may improve our tumor specific delivery of therapeutic agents, and minimize the bystander effects to normal cells from these cytotoxins. In such a way, we have the potential to deliver a cytotoxic payload to cancer cells in an antigen-specific manner.

1-43. (canceled)

44. A polymerized lipid nanoparticle comprising a polymerized lipid shell, wherein the polymerized lipid shell comprises at least 10% of polymerizable lipid, about 1-15% of negatively charged lipid, about 20-45% of neutrally charged molecules (such as cholesterol), and about 30% to 60% of zwitterionically charged lipid.

45. The polymerized lipid nanoparticle of claim 44, wherein the polymerized lipid shell comprises at least 15% to about 20% of 10,12-pentacosadiynoic acid derivatives and about 30% to about 40% of saturated phospholipids,

46. The polymerized lipid nanoparticle of claim 44, about 15% of C25 tail lipid and about 50% to about 55% of C18 tail lipid.

47. The polymerized lipid nanoparticle of claim 44, wherein the polymerizable lipid is a C25 tail lipid, the negatively charged lipid is a C18 tail lipid, and/or the zwitterionically charged lipid is a C18 tail lipid.

48. The polymerized lipid nanoparticle according to claim 44, wherein the polymerizable lipid is a diacetylenic lipid.

49. The polymerized lipid nanoparticle according to claim 44, further comprising at least one non-polymerizable lipid which is L-α-phosphatidylcholine; a PEG compound having a mass of 1000-5000 Daltons; 1,2-distearoyl-sn-glycero-3-phosphoethanolamine-N-[methoxy(polyethylene glycol)-2000]; or PE-PEG₂₀₀₀-biotin.

50. The polymerized lipid nanoparticle according to claim 44, wherein the polymerized lipid nanoparticle comprises a targeting agent, an imaging agent, a therapeutic agent, or a combination thereof.

51. The polymerized lipid nanoparticle of claim 50, wherein the targeting agent is selected from a group consisting of antibodies, ligands, proteins, peptides, carbohydrates, vitamins, nucleic acids, and combinations thereof.

52. The polymerized lipid nanoparticle of claim 50, wherein the targeting agent is specific to a cell surface molecule.

53. The polymerized lipid nanoparticle of claim 50, wherein the cell surface molecule is a cell membrane protein which includes structural proteins, cell adhesion molecules, membrane receptors, carrier proteins and channel proteins.

54. The polymerized lipid nanoparticle of claim 52, wherein the cell surface molecule is Activated Leukocyte Adhesion Molecule (CD-166), carbohydrate antigen 19-9 (CA19-9), Alphafetoprotein (AFP), Carcinoembryonic antigen (CEA), Ovarian cancer antigen (CA-125), breast cancer antigens (MUC-1 and epithelial tumor antigen (ETA)), Tyrosinase malignant melanoma antigen and Melanoma-associated antigen (MAGE), abnormal antigenic products of ras, p53, Ewing sarcoma antigen (CD-19), leukemia antigens (CD-99 and CD-117), Vascular Endothelial Growth Factor (VEGF), Epithelial Growth Factor Receptor (EGFR), Her2/neu, or prostate-specific membrane antigen (PSMA).

55. The polymerized lipid nanoparticle of claim 50, wherein the therapeutic agent is selected from the group comprising: antineoplastic agents, chemotherapeutic agents, blood products, biological response modifiers, anti-fungals, hormones, vitamins, peptides, anti-tuberculars, enzymes, anti-allergic agents, anti-coagulators, circulatory drugs, metabolic potentiators, antivirals, antianginals, antibiotics, antiinflammatories, antiprotozoans, antirheumatics, narcotics, opiates, cardiac glycosides, neuromuscular blockers, sedatives, local anesthetics, general anesthetics, radioactive compounds, monoclonal antibodies, genetic material, antisense nucleic acids such as siRNA or RNAi molecules, and prodrugs.

56. The polymerized lipid nanoparticle of claim 50, wherein the imaging agent is selected from the group consisting of magnetic resonance imaging contrast agents, including gadolinium, ultrasound imaging agents, and nuclear imaging agents, including Tc-99, In-111, Ga-67, Rh-105, I-123, I-124, Nd-147, Pm-151, Sm-153, Gd-159, Tb-161, Er-171, Re-186, Re-188, Tl-201, and Y-90.

57. The polymerized lipid nanoparticle of claim 50, wherein the polymerized lipid nanoparticle comprises a targeting agent and a therapeutic agent, wherein the polymerized lipid nanoparticle has a potency of at least 2 fold higher than conventional liposome pegylated preparation.

58. The polymerized lipid nanoparticle of claim 50, wherein the chemotherapeutic agent is a cytotoxic agent such as doxorubicin, irinotecan, cis-platin, topotecan, vincristine, mitomicin, paxlitaxol, and siRNA.

59. The polymerized lipid nanoparticle of claim 44, and further comprising a cys-diabody conjugated thereto.

60. The polymerized lipid nanoparticle of claim 59, wherein the cys-diabody is an anti-CA19.9 cys-diabody, or an anti-ALCAM cys-diabody.

61. The polymerized lipid nanoparticle according to claim 44, wherein the polymerized lipid nanoparticle is about 30 nm to about 200 nm in size.

62. A collection of polymerized lipid nanoparticles comprising a plurality of polymerized lipid nanoparticles according to claim 44.

63. A method of treating an individual in need thereof which comprises
administering to the individual a polymerized lipid nanoparticle according to claim 50.

64. The method of claim 63, wherein the polymerized lipid nanoparticle is internalized into the endosome compartment of a cell after 30 minutes of administration.

65. The method of claim 63, wherein the subject is being treated for cancer.


SEQUENCE LISTING (2 sequences)

SEQ ID NO: 1 - protein, 5 aa, Artificial Sequence
  Amino acid sequence based on the anti-CA19-9 diabody
GGCCG

SEQ ID NO: 2 - DNA, 48 bp, Artificial Sequence
  DNA fragment based on the anti-CA19-9 diabody
gaattctcaatgatgatgatgatgatgacccccacacccacctgcaga